(19) United States
(12) Patent Application Publication
(10) Pub. No.: US 2023/0340607 A1
(43) Pub. Date: Oct. 26, 2023

(54) COMPOSITIONS AND METHODS FOR DETECTING GENE FUSIONS OF RAD51AP1 AND DYRK4 AND FOR DIAGNOSING AND TREATING CANCER

(71) Applicant: UNIVERSITY OF PITTSBURGH-OF THE COMMONWEALTH SYSTEM OF HIGHER EDUCATION, Pittsburgh, PA (US)

(72) Inventors: Xiaosong WANG, Sewickley, PA (US); Chia Chia LIU, Pittsburgh, PA (US)

(21) Appl. No.: 18/016,188

(22) PCT Filed: Jul. 6, 2021
(86) PCT No.: PCT/US2021/040463

§ 371 Date: Jan. 13, 2023

Related U.S. Application Data
(60) Provisional application No. 63/050,983, filed on Jul. 13, 2020.

(51) Int. Cl. C12Q 1/6886; A61K 31/519

(52) U.S. Cl. CPC C12Q 1/6886; A61K 31/519; C12Q 2600/106; C12Q 2600/156; C12Q 2600/158

(57) ABSTRACT

Provided herein are compositions and methods for detecting RAD51AP1-DYRK4 fusions in a subject or tissue. In some embodiments, the subject or tissue is treated with an MEK inhibitor when a RAD51AP1-DYRK4 fusion is detected therein. Accordingly, included herein are methods for treating cancer in a subject using an MEK inhibitor and for identifying subjects that will be responsive to MEK inhibitor therapy.

### CROSS-REFERENCE TO RELATED APPLICATIONS

This application claims the benefit of U.S. Provisional Application No. 63/050,983, filed Jul. 13, 2020, which is expressly incorporated herein by reference.

### STATEMENT REGARDING FEDERALLY SPONSORED RESEARCH OR DEVELOPMENT

This invention was made with government support under grant numbers CA181368; CA183976 awarded by the National Institutes of Health; and grant number W81XWH-13-1-0431 awarded by the Department of Defense. The government has certain rights in the invention.

### FIELD

The present disclosure relates to the fields of detecting gene fusions and diagnosis and treatment of breast cancer.

### BACKGROUND

Estrogen receptor positive (ER+) breast cancer, also known as luminal breast cancer, can be classified into A and B intrinsic subtypes. Luminal B breast cancer accounts for 15-20% of all breast cancers (Yersal, O. & Barutca (2014)), and is the most common subtype in young women (Goksu, S.S. et al. (2014)). While the luminal A tumors can be effectively treated with endocrine therapy, the luminal B tumors are characterized by a higher proliferation index, more aggressive behavior, and endocrine resistance. Clinically, luminal B cancers show increased early relapse rates with a metastasis time pattern similar to basal-like breast cancer, and the treatment options are limited to concomitant endocrine and chemotherapy (Ades, F. et al. (2014)). Apart from higher growth factor signaling activities (Sotiriou, C. & Pusztai, L. (2009)), their underlying pathological molecular events remain unexplored. The recent transcriptome and genome sequencing studies have revealed a paucity of actionable oncogenic drivers in these tumors (Koboldt, D.C. et al. (2012)), which hinders the development of new diagnostic and treatment strategies.

What is needed are compositions and methods for detecting cancer-related gene fusions and for diagnosing and treating luminal and/or metastatic breast cancer. The compositions and methods disclosed herein address these and other needs.

### BRIEF SUMMARY

It is shown herein that RAD51AP1-DYRK4 fusions endow MEK inhibitor sensitivity in cancer cells. Accordingly, provided herein are new diagnostic and therapeutic strategies for breast tumors harboring RAD51AP1-DYRK4 fusions, wherein, in some embodiments, an MEK inhibitor is administered.

Provided herein are methods of diagnosing a subject with increased resistance to MEK inhibitors, comprising: obtaining a biological sample from the subject; and detecting an RAD51AP1-DYRK4 gene fusion in the sample, wherein the detection indicates the subject has increased sensitivity to an MEK inhibitor and the subject is diagnosed with increased sensitivity to an MEK inhibitor. In some embodiments, the RAD51AP1-DYRK4 gene fusion is selected from the group consisting of a E9-E2 fusion, a E8-E2 fusion, a E8s-E2 fusion, a E7-E2 fusion.

The method of detection can comprise contacting the biological sample with a reaction mixture comprising a probe specific for a fusion point in one of SEQ ID NO: 51, SEQ ID NO: 52 and SEQ ID NO: 53. The method of detection can alternatively or further comprise contacting the biological sample with a reaction mixture comprising two primers, wherein the first primer is complementary to a RAD51AP1 polynucleotide sequence and the second primer is complementary to a DYRK4 polynucleotide sequence, wherein the RAD51AP1-DYRK4 gene fusion is detectable by the presence of an amplicon generated by the first primer and the second primer. The method of detection can also comprise contacting the biological sample with a reaction mixture comprising two primers, wherein the first primer is complementary to a RADS51AP1 polynucleotide sequence and the second primer is complementary to a DYRK4 polynucleotide sequence, wherein hybridization of the two primers on a RAD51AP1-DYRK4 gene fusion sequence provides a detectable signal, and the RAD51AP1-DYRK4 gene fusion is detectable by the presence of the signal. In some embodiments, a first of the one or more primers is selected from the group consisting of SEQ ID NO: 5, SEQ ID NO: 7, SEQ ID NO: 25 and a second of the one or more primers is selected from the group consisting of SEQ ID NO: 6, SEQ ID NO: 8 and SEQ ID NO: 26. In some embodiments, the primers are SEQ ID NO: 5 and SEQ ID NO: 6. In some embodiments, the primers are SEQ ID NO: 7 and SEQ ID NO: 8. In some embodiments, the primers are SEQ ID NO: 25 and SEQ ID NO: 26.

The methods described herein can be used to detect a RAD51AP1-DYRK4 gene fusion in a subject that has a cancer, such as a breast cancer, including but not limited to a luminal B or metastatic breast cancer. The methods can further comprise administering to the subject a therapeutically effective amount of a MEK inhibitor.

Also included herein are methods of treating a cancer in a subject comprising: detecting a RAD51AP1-DYRK4 gene fusion in a sample obtained from the subject; and administering to the subject a therapeutically effective amount of a MEK inhibitor. The RAD51AP1-DYRK4 gene fusion can be selected from the group consisting of a E9-E2 fusion, a E8-E2 fusion, a E8s-E2 fusion, a E7-E2 fusion.

Further included are methods for detecting a RAD5 1AP1-DYRK4 gene fusion comprising: obtaining a biological sample from a subject; and detecting the fusion in the sample. In some embodiments, the detection can comprise contacting the biological sample with a reaction mixture comprising a probe specific for a fusion point sequence within one of SEQ ID NO: 51, SEQ ID NO: 52 and SEQ ID NO: 53. A detectable moiety can be covalently bonded to the probe, such as in a Nanostring assay. Kits comprising one or more probes are included, wherein each probe specifically hybridizes to a fusion point nucleotide sequence within a sequence selected from the group consisting of SEQ ID NO: 51, SEQ ID NO: 52 and SEQ ID NO: 53.

Further included are sequencing based methods such as transcriptome/genome sequencing methods or targeted sequencing for detecting a RAD51AP1-DYRK4 gene fusion comprising: obtaining a biological sample from a subject; and detecting the fusion variants in the sample through transcriptome/genome sequencing methods or targeted sequencing and bioinformatics detection tools.

Further included are protein-based methods known in the art, such as Mass spectrometry, immunohistochemistry, or western blot for detecting an RAD51AP1-DYRK4 protein product comprising: obtaining a biological sample from a subject; and detecting the fusion variant proteins in the sample through Mass spectrometry, immunohistochemistry, or western blot.

### DESCRIPTION OF DRAWINGS

FIGS. 1(A-C) shows the discovery and validation of RAD51AP1-DYRK4 as pathological chimerial transcript enriched in the luminal B and metastatic breast cancer. FIG. 1A shows the chimerical transcripts identified in the TCGA breast cancer samples are classified by their enrichment in the luminal B breast cancer, and then prioritized by the number of mean supporting reads and overall incidence. The ConSig scores for candidate fusions were depicted by the size of the dots. FIG. 1B shows schematic depicting the genomic location, strand, and exon-intron structure of the RAD51AP1 and DYRK4 loci, and the representative RAD51AP1-DYRK4 fusion variants. FIG. 1C shows RT-PCR validation of RAD51AP1- DYRK4 in ER+ breast cancer tissues using a forward primer in the first exon of RAD51AP1 and a reverse primer in the second exon of DYRK4. Representative RT-PCR gel images are shown in the upper panel, and representative chromatograms of each RAD51AP1-DYRK4 fusion variants are shown in the lower panel. *Weak RAD51AP1-DYRK4 expression.

FIGS. 2(A-D) shows the characteristics of RAD51AP1-DYRK4 overexpression in luminal breast cancer tissues. FIG. 2A depicts heat map showing the receptor status, Ki67 index, ESR1-CCDC170 or RAD51AP1-DYRK4 status (strong positivity), and wtRAD51AP1 overexpression in 200 ER+ breast cancer tissues. FIG. 2B shows RT-PCR analysis of RAD51AP1- DYRK4 in paired tumor (T) and adjacent normal tissues (N) from 12 strong positive cases reveals the tumor- specific expression of the RAD51AP1-DYRK4 transcript. WtRAD51AP1, wtDYRK4, and GAPDH were used as controls. FIG. 2C shows representative RT-PCR results of RAD51AP1-DYRK4, wtRAD51AP1, and wtDYRK4 in normal human tissue panels. FIG. 2D shows box plots comparing the Ki67 index for RAD51AP1-DYRK4 strong positive, weak positive, and negative breast tumors (upper panel), or comparing RAD51AP1-DYRK4 strong positive, RAD51AP1 high, and RAD51AP1 low fusion-negative tumors (lower panel). P-value was determined by t-test.

FIGS. 3(A-E) shows the characterization of the protein product of RAD51AP1-DYRK4 and its oncogenic potential. FIG. 3A shows schematic of RAD51AP1-DYRK4 fusion variants and their encoded proteins identified in breast cancer cell lines. ORFs are depicted in dark shades. FIG. 3B shows immunoblot analysis of T47D cells inducibly expressing RAD51AP1-DYRK4 (E9-E2 variant) or wtRAD51AP1 using an anti-RAD51AP1 polyclonal antibody. To verify the identity of the fusion protein bands, the engineered T47D cells are transfected with 5′RAD51AP1 siRNA designed to knockdown both RAD51AP1-DYRK4 and wtRAD51AP1, or the 3′RAD51AP1 siRNA designed to only inhibit the wtRAD51AP1. FIG. 3C shows that induction of RAD51AP1-DYRK4 ectopic expression (E9-E2 variant) in T47D cells resulted in a significant increase in cell motility. T47D cells inducibly expressing wtRAD51AP1 was used as control. FIG. 3D shows that ectopic expression of RAD51AP1-DYRK4 (E9-E2 variant) but not wtRAD51AP1 resulted in a significant increase in transendothelial migration of T47D cells. The T47D cells inducibly expressing E9-E2 or wtRAD51AP1 were treated with doxycycline and allowed to migrate through a confluent monolayer of human umbilical vein endothelial cells (HUVECs). FIG. 3E shows that silencing of wtRAD51AP1 does not affect RAD51AP1-DYRK4 driven cell motility. Left, specific knockdown of wtRAD51AP1 using two siRNAs against its 3′ region was verified by Western blotting. Cells are collected 48 hours following transfection with 10 nM 3′RADS51AP1 siRNAs or control siRNA. Right, transwell migration assay following induced E9-E2 overexpression and silencing of wtRAD51AP1. NIH 3T3 cells and 20%FBS are used as chemoattractant. (P<0.05, *, P<0.01**, P<0.001***).

FIGS. 4(A-D) shows that RAD51AP1-DYRK4 forms complex with MAP3K1 and activates MEK/ERK signaling. FIG. 4A shows the impact of RAD51AP1-DYRK4 or wtRAD51AP1 overexpression on the cellular signaling of the respective engineered T47D cells in the presence or absence of Matrigel extracellular matrix. The expression of RAD51AP1-DYRK4 or wtRAD51AP1 is induced using doxycycline (Dox) for 1 week. FIG. 4B shows increased activation of MEK/ERK in RAD51AP1-DYRK4 positive TCGA breast tumors (n=26) compared to fusion-negative LumB tumors overexpressing wtRAD51AP1 (n=36). The results are based on TCGA RPPA data. FIG. 4C shows immuno-precipitation analysis of T47D cells ectopically expressing RAD51AP1-DYRK4 (E9-E2) or wtRAD51AP1. Lysates from T47D cells ectopically expressing E9-E2 or wtRAD51AP1 were immune-precipitated using anti-RAD51AP1 or control IgG antibodies. The IP fractions were immunoblotted with indicated antibodies. WT, wtRAD51AP1. (D) QRT-PCR detecting RAD51AP1-DYRK4 or wtRAD51AP1 in the breast cancer cell lines used in this study.

FIGS. 5(A-E) shows the function of endogenous RAD51AP1-DYRK4 protein expressed in MDAMB361 luminal breast cancer cells. (A) Schematic of two 5′RAD51AP1 siRNAs targeting both fusion and wtRAD51AP1, two 3′RAD51AP1 siRNAs specifically targeting wtRAD51AP1, and two DYRK4 siRNAs targeting both fusion and wtDYRK4. (B) Detecting endogenous RAD51AP1-DYRK4 protein through western blot analysis of MDAMB361 cells treated with control siRNA (siCtrl), DYRK4 siRNAs, 5′RAD51AP1 siRNAs, or 3′RAD51AP1 siRNAs, using a RAD51AP1 polyclonal antibody. T47D cells inducibly expressing RAD51AP1-DYRK4 or wtRAD51AP1 are used as positive controls. (C) Detecting endogenous RAD51AP1-DYRK4 protein in the nuclear or cytoplasmic fractions of MDAMB361 cells treated with different siRNAs, using the RAD51AP1 polyclonal antibody. (D) Viability of MDAMB361 cells following treatment with indicated siRNAs (MTS assay). **p<0.01, ***p<0.001 (student’s T-test comparing to scrambled control siRNA at day 7). (E) The impact of silencing endogenous RAD51AP1-DYRK4 on MAP3K1/MEK/ERK signaling in MDAMB361 cells treated with indicated siRNAs. The RAD51AP1-DYRK4 protein was detected using the fusion-specific customized polyclonal antibody..

FIGS. 6(A-C) shows that RAD51AP1-DYRK4 endows increased sensitivity to the MEK inhibitor Trametinib. FIG. 6A shows that T47D cells inducibly overexpressing E9-E2 but not wtRAD51AP1 exhibit significantly increased sensitivity to Trametinib treatment as shown by clonogenic assays. Lapatinib alone or in combination with Trametinib did not show additional therapeutic benefits. FIG. 6B shows the effect of trametinib treatment in a panel of breast cancer cell lines with (bold font) or without RAD51AP1-DYRK4 overexpression (regular font) as shown by clonogenic assays. FIG. 6C shows that MDAMB361 cells overexpressing endogenous RAD51AP1-DYRK4 exhibit lapatinib resistance but highly sensitive to concomitant trametinib and lapatinib treatment as shown.

FIGS. 7(A-B) shows that RAD51AP1-DYRK4 attenuates compensatory feedback loop following MEK inhibition. (A) Western blot analysis of the engineered T47D cells inducibly overexpressing wtRAD51AP1 or E9E2 fusion harvested following trametinib or vehicle (DMSO) treatments. Cells were treated with Dox to induce wtRAD51AP1 or RAD51AP1-DYRK4 expression for one week, and then treated with 0.5 uM Trametinib or DMSO for 24 hours. (B) The mechanism engaged by RAD51AP1-DYRK4 to endow increased aggressiveness and confer sensitivity to MEK inhibition. RAD51AP1-DYRK4 forms complex with MAP3K1, activates MEK/ERK, and attenuates HER2/PI3K/AKT and JNK/c-Jun cascades under MEK inhibition. In contrast, wtRAD51AP1 overexpressing cancer cells show compensatory activation of the HER2/PI3K/AKT under MEK inhibition, leading to adaptive resistance to trametinib.

FIG. 8 shows the incidence of RAD51AP1-DYRK4 fusion variants (E9-E2, E8-E2, E8s-E2 and E7-E2) in different TCGA breast cancer clinical subtypes.

FIG. 9 shows that RAD51AP1-DYRK4 is preferentially detected in metastatic breast cancers in the MET500 and UPMC RNAseq datasets. RNAseq alignment were performed using Tophat v2.0.3 and gene fusions were detected using the fusion zoom pipeline.

FIG. 10 shows ROC analysis to determine the optimal cutoff of RAD51AP1-DYRK4 and wtRAD51AP1 overexpression based on RT-PCR band intensities. The RAD51AP1-DYRK4 and RAD51AP1 RT-PCR band intensities observed in breast tumor tissues were quantified using ImageJ software, and the ROCR module of the R statistical package was used to evaluate the optimal cutoffs for RAD51AP1-DYRK4 and RAD51AP1 overexpression.

FIG. 11 shows expression of RAD51AP1-DYRK4 transcripts in breast cancer cell lines detected by RT-PCR. RT-PCR of RAD51AP1-DYRK4 was done using a forward primer in the first exon of RAD51AP1 and a reverse primer in the second exon of DYRK4. The representative chromatograms of the fusion junction of each RAD51AP1-DYRK4 variant are shown in the lower panel. RT-PCR analysis of wtRAD51AP1 and wtDYRK4 was performed as controls. The HCC38 cell line shown here is a lineage passed in our lab that overexpress RAD51AP1-DYRK4, which is different from the HCC38 lineage newly purchased lineage from ATCC shown in FIG. 6B and FIG. 4D.

FIG. 12 shows expression of RAD51AP1-DYRK4 chimerical transcripts in triple-negative breast cancer tissues detected by RT-PCR. RT-PCR of RAD51AP1-DYRK4 was performed using a forward primer in the first exon of RAD51AP1 and a reverse primer in the second exon of DYRK4. RT-PCR analysis of wtRAD51AP1 and wtDYRK4 was performed as controls.

FIG. 13 shows Western blot analysis of T47D cells transiently expressing RAD51AP1-DYRK4 variants Flag-tagged at the 3′ end of the ΔRAD51AP1 ORF (*) or at the 3′ end of the DYRK4 ORF (#) using an anti-Flag antibody. The Flag-tagged wtRAD51AP1 and wtDYRK4 are used as controls. Solid arrows indicate the Flag-tagged ΔRAD51AP1 protein bands; white arrowheads indicate the Flag-tagged wtRAD51AP1 protein bands.

FIGS. 14(A-D) shows functional impact of ectopic RAD51AP1-DYRK4 expression in T47D breast cancer cells in vitro. FIG. 14A shows that RAD51AP1-DYRK4 did not significantly impact the proliferation of T47D breast cancer cells while wtRAD51AP1 overexpression had a repressing effect on cell proliferation. FIG. 14B shows ectopic expression of RAD51AP1-DYRK4 did not affect the T47D cell cycle progression, whereas wtRAD51AP1 increased the G1 cell population. FIGS. 14C-14D show the effect of RAD51AP1-DYRK4 ectopic expression on the (FIG. 14C) colony-formation and (FIG. 14D) anchorage-independent growth of T47D cells.

FIG. 15 shows Knockdown efficiency of siRNAs assessed by real-time PCR in MDAMB361 cells, using the primer pairs detecting wtRAD51AP1 (left), E9-E2 fusion (middle), or DYRK4(right).

FIG. 16 shows detecting endogenous RAD51AP1-DYRK4 protein in MDAMB361 cells using the customized antibody specifically against the DYRK4 frame-shift peptide. Endogenous RAD51AP1-DYRK4 protein was detected in the MDAMB361 fusion-positive cells treated with control siRNA (siCtrl), DYRK4 siRNAs, 5′RAD51AP1 siRNAs, or 3′RAD51AP1 siRNAs using the customized antibody against the DYRK4 frameshift peptide, as well as wtRAD51AP1 and wtDYRK4 polyclonal antibodies. T47D cells inducibly expressing E9-E2 fusion or wtRAD51AP1 are used as positive controls. The fusion-negative breast cancer cell line ZR-75-30 and HCC70, and benign breast epithelial cell line MCF12A are used as negative controls.

FIG. 17 shows TCGA tumors positive for MAP3K1 mutation or RAD51AP1-DYRK4, as well as the TCGA tumors overexpressing wtRAD51AP1. TCGA RNAseq and exome sequencing data revealed that MAP3K1 nonsynonymous mutations is rare in the breast tumor overexpressing RAD51AP1-DYRK4 or wild-type RAD51AP1.

### DETAILED DESCRIPTION

A previous study identified a recurrent ESR1-CCDC170 rearrangement in 6-8% of luminal B breast cancers which endows enhanced aggressiveness and reduced endocrine sensitivity (Veeraraghavan, J. et al. (2014)). This fusion was subsequently verified by several other studies (Fimereli, D. et al. (2018); Giltnane, J.M. et al. (2017); Matissek, K.J. et al. (2018); Hartmaier, R.J. et al. (2018)). In the present study, through a large-scale analysis of RNAseq data from The Cancer Genome Atlas (TCGA), a neoplastic chimerical transcript, RAD51AP1-DYRK4 was discovered. The transcript is silent in almost all human normal tissues but is markedly overexpressed in 3.6-9.5% of luminal breast cancer. More importantly, the overexpression of this chimera is associated with luminal B (7-17.5 %) and metastatic breast cancers (9-15%) and tends to be present in the tumors that are negative for ESR1-CCDC170 rearrangements. This disclosure investigated the molecular characteristics, clinical relevance, oncogenic and therapeutic role of RAD51AP1-DYRK4 in the more aggressive form of luminal breast cancers. It was discovered that RAD51AP1-DYRK4 endows enhanced activation of MEK/ERK signaling and increased aggressiveness of luminal breast cancers, and more importantly confers MEK inhibitor (MEKi) sensitivity via repressing MEKi- induced PI3K/AKT activation.

In some embodiments, the RAD51AP1-DYRK4 fusion polynucleotide encodes a c-terminal truncated RAD51AP1 protein fused to a small fragment of out- of-frame peptide from a DYRK4 protein, which leads to the loss of the RAD51 interacting domain. The truncation of RADS51AP1 and the addition of an outframe DYRK4 peptide resulting from this fusion may twist the biology of RAD51AP1. Herein, molecular evidence is provided showing that RAD51AP1-DYRK4 fusion expression is highly tumor-specific and is markedly enriched in ER+ luminal B breast tumors (7-18%) compared to luminal A tumors (3- 4%). In addition, RAD51AP1-DYRK4 fusion is preferentially overexpressed in 9-15% of metastatic tumors compared to 3.6-9.5% of primary tumors. Of note, the lower detection rate of RAD51AP1-DYRK4 fusion in TCGA tumors can be attributed to the short read-length (50 bp) and low sequencing depth of TCGA RNAseq data that limits the sensitivity of fusion detection. Ectopic expression of RAD51AP1-DYRK4, but not wild-type (wt) RAD51AP1, endows increased motility and transendothelial migration of luminal breast cancer cells, and the function of RAD51AP1-DYRK4 does not depend on the wild-type protein. Further, the endogenous RAD51AP1-DYRK4 protein was identified in fusion-positive cells, silencing of which leads to decreased cell viability.

The finding that RAD51AP1-DYRK4-mediated activation of MEK/ERK signaling regulates breast cancer migration and anoiksis resistance, emphasizes the significance and functional implications of RAD51AP1-DYRK4 fusion protein in breast cancer invasiveness and metastasis. More interestingly, these data show that RAD51AP1-DYRK4 fusion protein forms a complex with MAP3K1 and endows sensitivity to the MEK inhibitor (MEKi) Trametinib via attenuating compensatory PI3K-AKT activation. The present study further points out the importance of RAD51AP1-DYRK4 fusion protein in cytoplasmic signaling, due to the loss of RAD51 interacting domain and preferential localization to the cytoplasm.

Accordingly, in some aspects, disclosed herein is a method of detecting a fusion of a RAD51AP1 polynucleotide sequence and a DYRK4 polynucleotide sequence (referred to herein as a RAD51AP1-DYRK4 gene fusion), said method comprising obtaining a sample from a subject, and detecting whether the fusion is present in the sample. The fusion can be detected by contacting the sample with one or more primers specific for a RAD51AP1-DYRK4 fusion transcript, performing an amplification reaction, and detecting an amplification product or amplicon. The fusion can also be detected by transcriptome or genome sequencing, or targeted sequencing, or Nanostring assay, or Fluorescence In Situ Hybridization. This method can be used for detecting the RAD51AP1- DYRK4 gene fusion in a breast tissue sample and diagnosing a breast cancer (e.g., metastatic breast cancer or luminal B breast cancer). The method can also be used for determining if a breast cancer has an increased sensitivity to a MEK inhibitor (e.g., trametinib). In some aspects, disclosed herein is a method of treating a breast cancer in a subject, said method comprising detecting a fusion of a RAD51AP1 polynucleotide sequence and a DYRK4 polynucleotide sequence in a breast tissue sample obtained from the subject, and administering to the subject a therapeutically effective amount of a MEK inhibitor.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Qreplicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridg, UK), and self-sustained sequence replication (3SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002).

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is a breast cancer.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. In some embodiments, identity exists over the entirety of the compared nucleic acids or polypeptides. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Luminal B breast cancer” refers to a type of breast cancer that is hormone-receptor positive (estrogen-receptor and/or progesterone-receptor positive), and either HER2 positive or HER2 negative with high levels of Ki-67. Luminal B subtype tumors are more aggressive with a higher risk of early relapse with endocrine therapy. It has been unclear what drives these tumors to be more aggressive, and there are limited options for treating this type of cancer.

“Metastatic breast cancer”, also called stage IV cancer, refers to a breast cancer that has spread from one part of the body to another, most commonly the liver, brain, bones, or lungs.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. Unless otherwise specified, a “nucleotide sequence encoding an amino acid sequence” includes all nucleotide sequences that are degenerate versions of each other and that encode the same amino acid sequence. The nucleotide sequence that encodes a protein or an RNA may also include introns to the extent that the nucleotide sequence encoding the protein may in some version contain an intron(s).

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington’s Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polynucleotide” refers to a single or double stranded polymer composed of nucleotide monomers. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature. The most widely used target amplification procedure is PCR, first described for the amplification of DNA by Muliis et al. in U.S. Pat. No. 4,683,195 and Mullis in U.S. Pat. No. 4,683,202 and is well known to those of ordinary skill in the art.

A “primer” or “primer sequence” hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acids under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions. The primer will be completely or substantially complementary to a region of the target polynucleotide sequence to be copied. Therefore, under conditions conducive to hybridization, the primer will anneal to the complementary region of the target sequence. Upon addition of suitable reactants, including, but not limited to, a polymerase, nucleotide triphosphates, etc., the primer is extended by the polymerizing agent to form a copy of the target sequence. The primer may be single-stranded or alternatively may be partially double-stranded.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e., 5′ of the target sequence. The reverse primer has a nucleic acid sequence that is complementary to a sequence downstream, i.e., 3′ of the target sequence.

“Reporter probe” refers to a molecule used in an amplification reaction, typically for quantitative or real-time PCR analysis, as well as end-point analysis. Such reporter probes can be used to monitor the amplification of the target nucleic acid sequence. In some embodiments, reporter probes present in an amplification reaction are suitable for monitoring the amount of amplicon(s) produced as a function of time. Such reporter probes include, but are not limited to, the 5′-exonuclease assay (e.g., U.S. Pat. No. 5,538,848) various stem-loop molecular beacons (see for example, U.S. Pat. Nos. 6,103,476 and 5,925,517), stemless or linear beacons (see, e.g., WO 99/21881), PNA MOLECULAR BEACONS (see, e.g., U.S. Pat. Nos. 6,355,421 and 6,593,091), linear PNA beacons, non-FRET probes (see, for example, U.S. Pat. No. 6,150,097), SUNRISE/AMPLIFLUOR probes (U.S. Pat. No. 6,548,250), stem-loop and duplex Scorpion probes (U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB ECLIPSE probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901. Reporter probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “tissue” refers to a group or layer of similarly specialized cells which together perform certain special functions. The term “tissue” is intended to include, blood, blood preparations such as plasma and serum, bones, joints, muscles, smooth muscles, breast tissue, and organs.

The terms “treat,” “treating,” “treatment,” and grammatical variations thereof as used herein, include partially or completely alleviating, mitigating or reducing the intensity of one or more attendant symptoms of a disorder or condition and/or alleviating, mitigating or impeding one or more causes of a disorder or condition. In some instances, the terms “treat”, “treating”, “treatment” and grammatical variations thereof, refer to reducing tumor size in a subject, reducing cancer cell metastasis in a subject, and/or mitigation of a symptom of a cancer in a subject as compared with prior to treatment of the subject, as compared with the incidence of such symptom in a general or study population, or as compared to a subject or cancer tissue that does not have a RAD51AP1-DYRK4 fusion.

Prophylactic administrations are given to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is a reduction of tumor size. In some embodiments, a desired therapeutic result is a reduction of cancer metastasis. In some embodiments, a desired therapeutic result is a reduction of breast cancer, or a symptom of breast cancer. In some embodiments, a desired therapeutic result is the prevention of cancer relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired therapeutic effect is achieved following administration of multiple dosages of the composition to the subject over a period of days, weeks, or years.

### Methods of Detecting, Diagnosing and Treating

Disclosed herein are methods of detecting a fusion of a RAD51AP1 polynucleotide sequence and a DYRK4 polynucleotide sequence, said methods comprising obtaining a sample from a subject, and detecting whether the fusion is present in the sample. A fusion of a RAD51AP1 polynucleotide sequence and a DYRK4 polynucleotide sequence is also referred to herein as a RAD51AP1-DYRK4 gene fusion.

As used herein, “gene fusion” refers to a chimeric transcript resulting from the intergenic splicing of at least a portion of a first gene to a portion of a second gene, resulting in a chimeric mRNA. The point of transition between the sequence from the first gene in the fusion to the sequence from the second gene in the fusion is referred to as the “fusion point.” Methods for detecting a gene fusion include detection of the chimeric mRNA and detection of the resultant chimeric protein. Accordingly, it should be understood that a “gene fusion” or a “fusion of exons” includes a fusion of the mRNA transcripts of the exons described herein.

In some embodiments, a RAD51AP1-DYRK4 gene fusion is detected in a sample derived from a subject having breast cancer and the detection indicates that the breast cancer has increased sensitivity to an MEK inhibitor. As used herein, “increased sensitivity” means that the MEK inhibitor has a greater inhibitory effect on the cancer as compared to a control such as a cancer tissue or subject that does not have a RAD51AP1-DYRK4 gene fusion. In some embodiments, the increased sensitivity results in a lower effective dosage of the MEK inhibitor. In other embodiments, the increased sensitivity results in a shorter MEK inhibitor treatment time. In some embodiments, the increased sensitivity results in a greater reduction in tumor size, number and/or metastasis following treatment with an MEK inhibitor as compared to a control wherein the cancer tissue or subject does not have a RAD51AP1-DYRK4 gene fusion. Accordingly, the present invention includes methods of diagnosing a breast cancer having increased sensitivity to a MEK inhibitor.

Also disclosed herein is a method of treating a breast cancer in a subject, said method comprising detecting a fusion of a RAD51AP1 polynucleotide sequence and a DYRK4 polynucleotide sequence in a breast tissue sample obtained from the subject, and administering to the subject a therapeutically effective amount of a MEK inhibitor.

“RAD51AP1” or “RAD51 Associated Protein 1” refers herein to a polypeptide that synthesizes and hydrolyzes cyclic adenosine 5′-diphosphate-ribose, and in humans, is encoded by the RAD51AP1 gene. In some embodiments, the RAD51AP1 polypeptide or polynucleotide is that identified in one or more publicly available databases as follows: HGNC: 16956, Entrez Gene: 10635, Ensembl: ENSG00000111247, OMIM: 603070, and UniProtKB: Q96B01. In some embodiments, the RAD51AP1 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. In some embodiments, the RAD51AP1 polypeptide is an isoform of SEQ ID NO:1. In some embodiments, the RAD51AP1 polypeptide is a ortholog of SEQ ID NO:1. The RADS51AP1 polypeptide of SEQ ID NO: 1 may represent an immature or pre-processed form of mature RADS51AP1, and accordingly, included herein are mature or processed portions of the RAD51AP1 polypeptide in SEQ ID NO: 1. In some embodiments, the RAD51AP1 polypeptide is encoded by RAD51AP1 polynucleotide comprising the sequence of SEQ ID NO: 2, or a polynucleotide sequence having at or greater than about 80%, about 85%, about 90%, about 95%, or about 98% homology with SEQ ID NO: 2, or a polynucleotide comprising a portion of SEQ ID NO: 2. As used herein, the term “RAD51AP1 polynucleotide sequence” refers to any polynucleotide sequence that encodes a RAD51AP1 polypeptide, or any fragment thereof.

In some embodiments, the RAD51AP1 polynucleotide is an mRNA transcript comprising a sequence that corresponds to RAD51AP1 exon 1 polynucleotide having a sequence of SEQ ID NO: 27, or a polynucleotide having at or greater than about 80%, about 85%, about 90%, about 95%, or about 98% homology with SEQ ID NO: 27, or a polynucleotide comprising a portion of SEQ ID NO: 27. In some embodiments, the RAD51AP1 polynucleotide is an mRNA transcript comprising a sequence that corresponds to a RAD51AP1 exon 2 polynucleotide having a sequence of SEQ ID NO: 28, or a polynucleotide having at or greater than about 80%, about 85%, about 90%, about 95%, or about 98% homology with SEQ ID NO: 28, or a polynucleotide comprising a portion of SEQ ID NO: 28. In some embodiments, the RAD51AP1 polynucleotide is an mRNA transcript comprising a sequence that corresponds to a RAD51AP1 exon 3 polynucleotide having a sequence of SEQ ID NO: 29, or a polynucleotide having at or greater than about 80%, about 85%, about 90%, about 95%, or about 98% homology with SEQ ID NO: 29, or a polynucleotide comprising a portion of SEQ ID NO: 29. In some embodiments, the RAD51AP1 polynucleotide is an mRNA transcript comprising a sequence that corresponds to a RAD51AP1 exon 4 polynucleotide having a sequence of SEQ ID NO: 30, or a polynucleotide having at or greater than about 80%, about 85%, about 90%, about 95%, or about 98% homology with SEQ ID NO: 30, or a polynucleotide comprising a portion of SEQ ID NO: 30. In some embodiments, the RAD51AP1 polynucleotide is an mRNA transcript comprising a sequence that corresponds to a RAD51AP1 exon 5 polynucleotide having a sequence of SEQ ID NO: 31, or a polynucleotide having at or greater than about 80%, about 85%, about 90%, about 95%, or about 98% homology with SEQ ID NO: 31, or a polynucleotide comprising a portion of SEQ ID NO: 31. In some embodiments, the RAD51AP1 polynucleotide is an mRNA transcript comprising a sequence that corresponds to a RAD51AP1 exon 6 polynucleotide having a sequence of SEQ ID NO: 32, or a polynucleotide having at or greater than about 80%, about 85%, about 90%, about 95%, or about 98% homology with SEQ ID NO: 32, or a polynucleotide comprising a portion of SEQ ID NO: 32. In some embodiments, the RAD51AP1 polynucleotide is an mRNA transcript comprising a sequence that corresponds to a RAD51AP1 exon 8 polynucleotide having a sequence of SEQ ID NO: 33, or a polynucleotide having at or greater than about 80%, about 85%, about 90%, about 95%, or about 98% homology with SEQ ID NO: 33, or a polynucleotide comprising a portion of SEQ ID NO: 33. In some embodiments, the RAD51AP1 polynucleotide is an mRNA transcript comprising a sequence that corresponds to a RAD51AP1 exon 8 s polynucleotide having a sequence of SEQ ID NO: 34, or a polynucleotide having at or greater than about 80%, about 85%, about 90%, about 95%, or about 98% homology with SEQ ID NO: 34, or a polynucleotide comprising a portion of SEQ ID NO: 34. In some embodiments, the RAD51AP1 polynucleotide is an mRNA transcript comprising a sequence that corresponds to a RAD51AP1 exon 9 polynucleotide having a sequence of SEQ ID NO: 35, or a polynucleotide having at or greater than about 80%, about 85%, about 90%, about 95%, or about 98% homology with SEQ ID NO: 35, or a polynucleotide comprising a portion of SEQ ID NO: 35. In some embodiments, the RAD51AP1 polynucleotide is an mRNA transcript comprising a sequence that corresponds to a RAD51AP1 exon 10 polynucleotide having a sequence of SEQ ID NO: 36, or a polynucleotide having at or greater than about 80%, about 85%, about 90%, about 95%, or about 98% homology with SEQ ID NO: 36, or a polynucleotide comprising a portion of SEQ ID NO: 36.

“DYRK4” or “Dual Specificity Tyrosine Phosphorylation Regulated Kinase 4” refers herein to a polypeptide that synthesizes and hydrolyzes cyclic adenosine 5′-diphosphate-ribose, and in humans, is encoded by the DYRK4 gene. In some embodiments, the DYRK4 polypeptide is that identified in one or more publicly available databases as follows: HGNC: 3095, Entrez Gene: 8798, Ensembl: ENSG00000010219, OMIM: 609181, and UniProtKB: Q9NR20. In some embodiments, the DYRK4 polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. In some embodiments, the DYRK4 polypeptide is an isoform of SEQ ID NO:3. In some embodiments, the DYRK4 polypeptide is a ortholog of SEQ ID NO:3. The DYRK4 polypeptide of SEQ ID NO: 3 may represent an immature or pre-processed form of mature DYRK4, and accordingly, included herein are mature or processed portions of the DYRK4 polypeptide in SEQ ID NO: 3. In some embodiments, the DYRK4 polypeptide is encoded by DYRK4 polynucleotide comprising the sequence of SEQ ID NO: 4, or a polynucleotide sequence having at or greater than about 80%, about 85%, about 90%, about 95%, or about 98% homology with SEQ ID NO: 4, or a polynucleotide comprising a portion of SEQ ID NO: 4. As used herein, the term “DYRK4 polynucleotide sequence” refers to any polynucleotide sequence that encodes a DYRK4 polypeptide, or any fragment thereof.

In some embodiments, the DYRK4 polynucleotide is an mRNA transcript comprising a sequence that corresponds to a DYRK4 exon 1 polynucleotide having a sequence of SEQ ID NO: 37, or a polynucleotide having at or greater than about 80%, about 85%, about 90%, about 95%, or about 98% homology with SEQ ID NO: 37, or a polynucleotide comprising a portion of SEQ ID NO: 37. In some embodiments, the DYRK4 polynucleotide is an mRNA transcript comprising a sequence that corresponds to a DYRK4 exon 2 polynucleotide having a sequence of SEQ ID NO: 38, or a polynucleotide having at or greater than about 80%, about 85%, about 90%, about 95%, or about 98% homology with SEQ ID NO: 38, or a polynucleotide comprising a portion of SEQ ID NO: 38. In some embodiments, the DYRK4 polynucleotide is an mRNA transcript comprising a sequence that corresponds to a DYRK4 exon 3 polynucleotide having a sequence of SEQ ID NO: 39, or a polynucleotide having at or greater than about 80%, about 85%, about 90%, about 95%, or about 98% homology with SEQ ID NO: 39, or a polynucleotide comprising a portion of SEQ ID NO: 39. In some embodiments, the DYRK4 polynucleotide is an mRNA transcript comprising a sequence that corresponds to a DYRK4 exon 4 polynucleotide having a sequence of SEQ ID NO: 40, or a polynucleotide having at or greater than about 80%, about 85%, about 90%, about 95%, or about 98% homology with SEQ ID NO: 40, or a polynucleotide comprising a portion of SEQ ID NO: 40. In some embodiments, the DYRK4 polynucleotide is an mRNA transcript comprising a sequence that corresponds to a DYRK4 exon 5 polynucleotide having a sequence of SEQ ID NO: 41, or a polynucleotide having at or greater than about 80%, about 85%, about 90%, about 95%, or about 98% homology with SEQ ID NO: 41, or a polynucleotide comprising a portion of SEQ ID NO: 41. In some embodiments, the DYRK4 polynucleotide is an mRNA transcript comprising a sequence that corresponds to a DYRK4 exon 6 polynucleotide having a sequence of SEQ ID NO: 42, or a polynucleotide having at or greater than about 80%, about 85%, about 90%, about 95%, or about 98% homology with SEQ ID NO: 42, or a polynucleotide comprising a portion of SEQ ID NO: 42. In some embodiments, the DYRK4 polynucleotide is an mRNA transcript comprising a sequence that corresponds to a DYRK4 exon 7 polynucleotide having a sequence of SEQ ID NO: 43, or a polynucleotide having at or greater than about 80%, about 85%, about 90%, about 95%, or about 98% homology with SEQ ID NO: 42, or a polynucleotide comprising a portion of SEQ ID NO: 42. In some embodiments, the DYRK4 polynucleotide is an mRNA transcript comprising a sequence that corresponds to a DYRK4 exon 8 polynucleotide having a sequence of SEQ ID NO: 44, or a polynucleotide having at or greater than about 80%, about 85%, about 90%, about 95%, or about 98% homology with SEQ ID NO: 44, or a polynucleotide comprising a portion of SEQ ID NO: 44. In some embodiments, the DYRK4 polynucleotide is an mRNA transcript comprising a sequence that corresponds to a DYRK4 exon 9 polynucleotide having a sequence of SEQ ID NO: 45, or a polynucleotide having at or greater than about 80%, about 85%, about 90%, about 95%, or about 98% homology with SEQ ID NO: 45, or a polynucleotide comprising a portion of SEQ ID NO: 45. In some embodiments, the DYRK4 polynucleotide is an mRNA transcript comprising a sequence that corresponds to a DYRK4 exon 10 polynucleotide having a sequence of SEQ ID NO: 46, or a polynucleotide having at or greater than about 80%, about 85%, about 90%, about 95%, or about 98% homology with SEQ ID NO: 46, or a polynucleotide comprising a portion of SEQ ID NO: 46. In some embodiments, the DYRK4 polynucleotide is an mRNA transcript comprising a sequence that corresponds to a DYRK4 exon 11 polynucleotide having a sequence of SEQ ID NO: 47, or a polynucleotide having at or greater than about 80%, about 85%, about 90%, about 95%, or about 98% homology with SEQ ID NO: 47, or a polynucleotide comprising a portion of SEQ ID NO: 47. In some embodiments, the DYRK4 polynucleotide is an mRNA transcript comprising a sequence that corresponds to a DYRK4 exon 12 polynucleotide having a sequence of SEQ ID NO: 48, or a polynucleotide having at or greater than about 80%, about 85%, about 90%, about 95%, or about 98% homology with SEQ ID NO: 48, or a polynucleotide comprising a portion of SEQ ID NO: 48. In some embodiments, the DYRK4 polynucleotide is an mRNA transcript comprising a sequence that corresponds to a DYRK4 exon 13 polynucleotide having a sequence of SEQ ID NO: 49, or a polynucleotide having at or greater than about 80%, about 85%, about 90%, about 95%, or about 98% homology with SEQ ID NO: 49, or a polynucleotide comprising a portion of SEQ ID NO: 49. In some embodiments, the DYRK4 polynucleotide is an mRNA transcript comprising a sequence that corresponds to a DYRK4 exon 14 polynucleotide having a sequence of SEQ ID NO: 50, or a polynucleotide having at or greater than about 80%, about 85%, about 90%, about 95%, or about 98% homology with SEQ ID NO: 50, or a polynucleotide comprising a portion of SEQ ID NO: 50. In some embodiments, the DYRK4 polynucleotide is an mRNA transcript comprising a sequence that corresponds to a DYRK4 exon 15 polynucleotide having a sequence of SEQ ID NO: 51, or a polynucleotide having at or greater than about 80%, about 85%, about 90%, about 95%, or about 98% homology with SEQ ID NO: 51, or a polynucleotide comprising a portion of SEQ ID NO: 51.

It should be understood that the term “fusion” as used herein refers to a polynucleotide or polypeptide made by joining parts of two previously independent polynucleotides or polypeptides of RAD51AP1 and DYRK4. In some embodiments, a fusion is formed by joining parts of two previously independent genes through translocation, interstitial deletion, or chromosomal inversion. Accordingly, “a fusion of a RAD51AP1 polynucleotide sequence and a DYRK4 polynucleotide sequence” refers herein to a fusion of a RAD51AP1 DNA sequence and a DYRK4 DNA sequence, a fusion mRNA transcribed from the fusion DNA, or a fusion mRNA that is the result of intergenic splicing. “RAD51AP1-DYRK4 polynucleotide fusion” is used interchangeably herein with “fusion of a RAD51AP1 polynucleotide sequence and a DYRK4 polynucleotide sequence.” “RAD51AP1-DYRK4 fusion” refers to a “RAD51AP1-DYRK4 polynucleotide fusion” and/or a “RAD51AP1-DYRK4 polypeptide fusion.”

In some embodiments, the phrase “a fusion of a RAD51AP1 polynucleotide sequence and a DYRK4 polynucleotide sequence” herein refers to a fusion of any RAD51AP1 exon or exon mRNA transcript and any DYRK4 exon or exon mRNA transcript (e.g. a fusion of any RAD51AP1 exon or exons with any DYRK4 exon or exons). In some embodiments, the fusion described herein is a fusion containing a fusion exon junction of any of the exons, or exon transcripts, 2-9 of a RAD51AP1 polynucleotide with any of the exons, or exon transcripts, 2-15 of a DYRK4 polynucleotide. In some embodiments, the fusion is: a fusion of exons, or exon transcripts, 2-9 of a RAD51AP1 polynucleotide (having a portion of exon 1) with exons, or exon transcripts, 2-15 of a DYRK4 polynucleotide (referred to herein as an “E9-E2 fusion”); a fusion of exons, or exon transcripts, 2-8 of a RAD51AP1 polynucleotide (having a portion of exon 1) with exons, or exon transcripts, 2-15 of a DYRK4 polynucleotide (referred to herein as an “E8-E2 fusion”); a fusion of exons, or exon transcripts, 2-8 s of a RAD51AP1 polynucleotide (having a portion of exon 1) with exons, or exon transcripts, 2-15 of a DYRK4 polynucleotide (referred to herein as an “E8s-E2 fusion”); a fusion of exons, or exon transcripts, 2-7 of a RAD51AP1 polynucleotide (having a portion of exon 1) with exons, or exon transcripts, 2-15 of a DYRK4 polynucleotide (referred to herein as an “E7-E2 fusion”). As used herein, the term “E8s” refers to an alternative splice variant of DYRK4 exon 8. In one embodiment, an E8s exon has a sequence of SEQ ID NO: 34. In some embodiments, the RAD51AP1-DYRK4 fusion comprises a RAD51AP1 exon mRNA transcript that corresponds to SEQ ID NO: 55, SEQ ID NO:56 or SEQ ID NO: 57.

In one example, the fusion of a RAD51AP1 polynucleotide sequence and a DYRK4 polynucleotide sequence disclosed herein encodes a RAD51AP1 protein fused to a fragment of a protein sequence of DYRK4. In some embodiments, the RAD51AP1 protein has its C-terminal region truncated. In some embodiments, the fragment of the protein sequence of DYRK4 is an out-of-frame protein fragment. In some embodiments, the fusion polynucleotide sequence described herein encodes a C-terminally truncated RAD51AP1 protein fused to a fragment of an out-of-frame DYRK4 protein sequence.

The fusions described herein can be detected by contacting the sample with one or more primers specific for the fusion, performing an amplification reaction, and detecting an amplification product or amplicon. It should be understood and herein contemplated that the term “amplification reaction” of polynucleotide as used herein means the use of an amplification reaction (e.g., PCR) to increase the concentration of a particular nucleic acid sequence within a mixture of nucleic acid sequences. The term “PCR” as used herein refers to the polymerase chain reaction, a laboratory technique used to make multiple copies of a segment of a polynucleotide, as is well- known in the art. The term “PCR” includes all forms of PCR, such as real-time PCR, quantitative reverse transcription PCR (qRT-PCR), multiplex PCR, nested PCR, hot start PCR, or GC-Rich PCR. In some embodiments, the amplification reaction is real-time PCR. Exemplary procedures for real-time PCR can be found in “Quantitation of DNA/RNA Using Real-Time PCR Detection” published by Perkin Elmer Applied Biosystems (1999) and to PCR Protocols (Academic Press New York, 1989), incorporated by reference herein in their entireties. The amplification reaction can also be a loop-mediated isothermal amplification (LAMP), a reaction at a constant temperature using primers recognizing the distinct regions of target DNA for a highly specific amplification reaction. In some embodiments, the RAD51AP1-DYRK4 polynucleotide fusion disclosed herein is detected by methods such as the Nanostring nCounter assay which directly measures target molecules without PCR amplification using ghost probes against one fusion partner gene, and reporter probes against the other fusion partner gene. In some embodiments, a fusion protein encoded by the fusion polynucleotide disclosed herein is detected by one or more protein detection assays including, for example, Western blotting, immunoblotting, ELISA, immunohistochemistry, or an electrophoresis method (e.g., SDS-PAGE).

The fusion can also be detected by any RNA or protein-based methods known in the art, such as Nanostring assay or whole transcriptome, or targeted transcriptome or genome sequencing, or fluorescence in situ hybridization, or immunohistochemistry, or western blot.

In some embodiments, the one or more primers or Nanostring probes comprise the sequence of SEQ ID NO: 5 or SEQ ID NO: 7, or a polynucleotide sequence having at or greater than about 80%, about 85%, about 90%, about 95%, or about 98% homology with SEQ ID NO: 5 or SEQ ID NO: 7, or a polynucleotide comprising a portion of SEQ ID NO: 5 or SEQ ID NO: 7. In some embodiments, the one or more primers comprise the polynucleotide sequence of SEQ ID NO: 5 or SEQ ID NO: 7 or a fragment thereof.

In some embodiments, the one or more PCR primers or Nanostring probes comprise the sequence of SEQ ID NO: 6 or SEQ ID NO: 8, or a polynucleotide sequence having at or greater than about 80%, about 85%, about 90%, about 95%, or about 98% homology with SEQ ID NO: 6 or SEQ ID NO: 8, or a polynucleotide comprising a portion of SEQ ID NO: 6 or SEQ ID NO: 8. In some embodiments, the one or more primers comprise the polynucleotide sequence of SEQ ID NO: 6 or SEQ ID NO: 8 or a fragment thereof.

As used herein, the term “detecting” refers to detection of a level of a fusion (e.g., the fusion of a RAD51AP1 polynucleotide sequence and a DYRK4 polynucleotide) that is at least about 5% (e.g., at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1000%, at least about 2000%, at least about 3000%, or at least about 5000%) or at least about 5 times (e.g., at least about 6 times, at least about 7 times, at least about 8 times, at least about 9 times, at least about 10 times, at least about 20 times, at least about 30 times, at least about 40 times, at least about 50 times, or at least about 100 times) higher as compared to a sample from a subject in general or a study population (e.g., healthy control).

In certain embodiments the primers are used in DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, regular PCR, real-time PCR, DNA sequencing, DNA extension, DNA polymerization, RNA transcription, and reverse transcription. Techniques and conditions that amplify the primer in a sequence specific manner are preferred. In certain embodiments, the primers are used for the DNA or RNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. In some embodiments, the primers are used for gene array analysis. Typically, the disclosed primers hybridize with a region of the disclosed nucleic acids (e.g., RADS51AP1 or DYRK4) or they hybridize with the complement of the nucleic acids or complement of a region of the nucleic acids.

In some embodiments, the “sample” referred to herein is a tissue sample. In some embodiments, the sample is a breast tissue sample. In some embodiments, the breast tissue is cancerous. Included herein are methods that comprise detection of an increased amount of the RAD51AP1-DYRK4 fusion in a breast tissue sample as compared to a control, wherein the control can be a normal breast tissue or any normal tissue other than testis tissue, and wherein the control can be obtained from the same subject or a different subject. In some embodiments, the control is a level or amount of the RAD51AP1-DYRK4 fusion in a general or study population. In some embodiments, the control is a tissue sample that does not have a RAD51AP1-DYRK4 fusion. In some embodiments, the cancerous breast tissue exhibits an increased amount of the fusion of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a control, or at least about a 2-fold, or at least about a 3-fold, or at least about a 4-fold, or at least about a 5-fold, or at least about a 10-fold, at least about a 20-fold, at least about a 50-fold, at least about a 100-fold, at least about a 500-fold, or at least about a 1000-fold as compared to a control.

It should be understood and herein contemplated that detection of the RAD51AP1-DYRK4 fusion or an increase in the amount of the RAD51AP1-DYRK4 fusion as compared to a control indicates an increased sensitivity of the tissue sample, cancer cell or tumor to a MEK inhibitor. In some embodiments, the increased sensitivity of a cancer cell or tumor refers to a more significant decrease in tumor growth, a larger decrease in tumor volume or size, a faster clearance of tumor, an increase in cancer cell death, a decrease in cell migration, metastasis, and/or proliferation, a decrease in MAP3K1 protein level and/or a decrease in JNK-JUN phosphorylation level in the cancer cell in response to the same or a lower dose of a MEK inhibitor as compared to a control cancer cell or tumor, wherein the control tumor or cancer cell does not have the RAD51AP1-DYRK4 fusion disclosed herein. In some embodiments, the tumor or cancer cell comprising the RAD51AP1-DYRK4 fusion exhibits an increased sensitivity to a MEK inhibitor of at least about at least about 10%, at least about 20%, or at least about 30%, or at least about 40%, or at least about 50%, or at least about 60%, or at least about 70%, or at least about 80%, or at least about 90% or at least about 100%, or an increased sensitivity to a MEK inhibitor of at least about a 2-fold, or at least about a 3-fold, or at least about a 4-fold, or at least about a 5-fold, or at least about a 10-fold, at least about a 20-fold, at least about a 50-fold, at least about a 100-fold, or at least about a 500-fold as compared to a control.

Accordingly, included in the present invention are methods of treating a cancer comprising detecting a fusion of a RAD51AP1 polynucleotide sequence and a DYRK4 polynucleotide sequence in a breast tissue sample obtained from the subject and administering to the subject a therapeutically effective amount of a MEK inhibitor.

As used herein, “MEK inhibitor” refers to an inhibitor of MEK1 and/or MEK2. “MEK1” or “Mitogen-activated protein kinase kinase 1” is also known as MAP2K1 or MAPKK 1 and is a dual specificity protein kinase which acts as a component of the MAP kinase signal transduction pathway. Binding of extracellular ligands such as growth factors, cytokines and hormones to their cell-surface receptors activates RAS and this initiates RAF1 activation. In some embodiments, the MEK1 polypeptide is that identified in one or more publicly available databases as follows: HGNC: 6840, Entrez Gene: 5604, Ensembl: ENSG000000169032, OMIM: 176872, and UniProtKB: Q02750. In some embodiments, the MEK1 polypeptide comprises the sequence of SEQ ID NO: 9, or a polypeptide sequence having at or greater than about 80%, about 85%, about 90%, about 95%, or about 98% homology with SEQ ID NO: 9, or a polypeptide comprising a portion of SEQ ID NO: 9. The MEK1 polypeptide of SEQ ID NO: 9 may represent an immature or pre-processed form of mature MEK1, and accordingly, included herein are mature or processed portions of the MEK1 polypeptide in SEQ ID NO: 9. “MEK2” or “Mitogen-activated protein kinase kinase 2” is also known as MAP2K2 or MAPKK 2 and catalyzes the concomitant phosphorylation of a threonine and a tyrosine residue in a Thr-Glu-Tyr sequence located in MAP kinases. In some embodiments, the MEK2 polypeptide is that identified in one or more publicly available databases as follows: HGNC: 6842, Entrez Gene: 5605, Ensembl: ENSG000000126934, OMIM: 601263, and UniProtKB: P36507. In some embodiments, the MEK2 polypeptide comprises the sequence of SEQ ID NO: 9, or a polypeptide sequence having at or greater than about 80%, about 85%, about 90%, about 95%, or about 98% homology with SEQ ID NO: 10, or a polypeptide comprising a portion of SEQ ID NO: 10. The MEK1 polypeptide of SEQ ID NO: 10 may represent an immature or pre-processed form of mature MEK1, and accordingly, included herein are mature or processed portions of the MEK1 polypeptide in SEQ ID NO: 10.

“MEK Inhibitors” refers to compositions that inhibit expression or of activity of an MEK polypeptide. Inhibitors are agents that, e.g., inhibit expression, partially or totally block activity, decrease, prevent, delay activation, inactivate, desensitize, or down regulate the activity of the MEK polypeptide. In some embodiments, samples or assays comprising the MEK polypeptide that are treated with an inhibitor are compared to control samples without the inhibitor to examine the extent of effect. Control samples (untreated with the inhibitor) can be assigned a relative activity value of 100%. Inhibition of the MEK polypeptide is achieved when the activity value relative to the control is about 80%, optionally 50% or 25, 10%, 5% or 1%. In some embodiments, the MEK inhibitor is trametinib, cobimetinib, binimetinib, selumetinib, Refametinib, Pimasertib, RO4987655, RO5126766, WX-554, HL-085, PD-325901, PD184352, AZD8330, TAK-733 or GDC-0623. In some embodiments, the MEK inhibitor is selected from the group consisting of trametinib, cobimetinib, binimetinib, selumetinib, Refametinib, Pimasertib, RO4987655, RO5126766, WX-554, HL-085, PD-325901, PD184352, AZD8330, TAK-733 and GDC-0623. In some embodiments, the MEK inhibitor is trametinib having the below chemical structure.

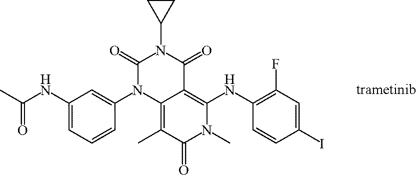 [SMILES: CC(=O)Nc1cccc(-n2c(=O)n(C3CC3)c(=O)c3c(Nc4ccc(I)cc4F)n(C)c(=O)c(C)c32)c1]

In some embodiments, the MEK inhibitor is cobimetinib having the below chemical structure.

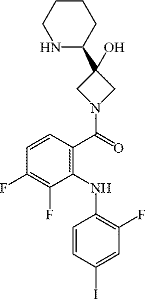 [SMILES: O=C(c1ccc(F)c(F)c1Nc1ccc(I)cc1F)N1CC(O)([C@@H]2CCCCN2)C1]

In some embodiments, the MEK inhibitor is binimetinib having the below chemical structure.

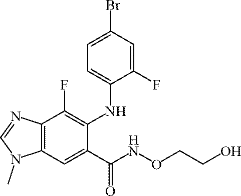 [SMILES: Cn1cnc2c(F)c(Nc3ccc(Br)cc3F)c(C(=O)NOCCO)cc21]

In some embodiments, the MEK inhibitor is selumetinib having the below chemical structure.

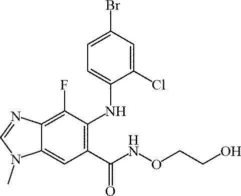 [SMILES: Cn1cnc2c(F)c(Nc3ccc(Br)cc3Cl)c(C(=O)NOCCO)cc21]

In some embodiments, the MEK inhibitor is Refametinib having the below chemical structure.

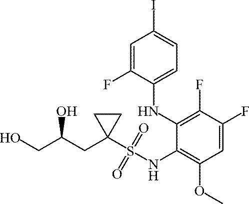 [SMILES: COc1cc(F)c(F)c(Nc2ccc(I)cc2F)c1NS(=O)(=O)C1(C[C@H](O)CO)CC1]

In some embodiments, the MEK inhibitor is Pimasertib having the below chemical structure.

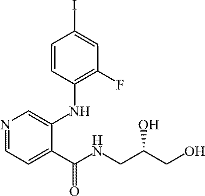 [SMILES: O=C(NC[C@H](O)CO)c1ccncc1Nc1ccc(I)cc1F]

In some embodiments, the MEK inhibitor is RO4987655 having the below chemical structure.

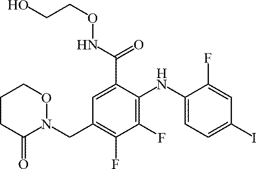 [SMILES: O=C(NOCCO)c1cc(CN2OCCCC2=O)c(F)c(F)c1Nc1ccc(I)cc1F]

In some embodiments, the MEK inhibitor is RO5126766 having the below chemical structure.

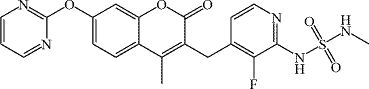 [SMILES: CNS(=O)(=O)Nc1nccc(Cc2c(C)c3ccc(Oc4ncccn4)cc3oc2=O)c1F]

In some embodiments, the MEK inhibitor is PD-325901 having the below chemical structure.

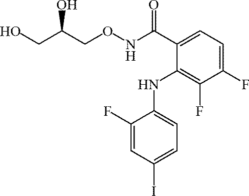 [SMILES: O=C(NOC[C@H](O)CO)c1ccc(F)c(F)c1Nc1ccc(I)cc1F]

In some embodiments, the MEK inhibitor is PD184352 having the below chemical structure.

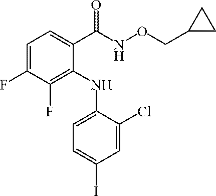 [SMILES: O=C(NOCC1CC1)c1ccc(F)c(F)c1Nc1ccc(I)cc1Cl]

In some embodiments, the MEK inhibitor is AZD8330 having the below chemical structure.

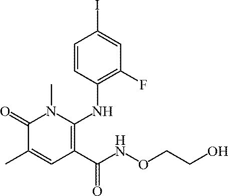 [SMILES: Cc1cc(C(=O)NOCCO)c(Nc2ccc(I)cc2F)n(C)c1=O]

In some embodiments, the MEK inhibitor is TAK-733 having the below chemical structure.

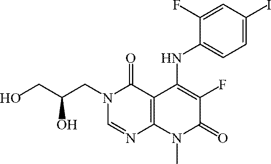 [SMILES: Cn1c(=O)c(F)c(Nc2ccc(I)cc2F)c2c(=O)n(C[C@@H](O)CO)cnc21]

In some embodiments, the MEK inhibitor is GDC-0623 having the below chemical structure.

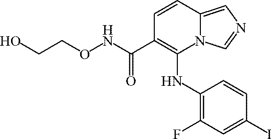 [SMILES: O=C(NOCCO)c1ccc2cncn2c1Nc1ccc(I)cc1F]

In some embodiments, subject has a cancer. The cancer can be any of breast cancer, prostate cancer, ovarian cancer, cervical cancer, skin cancer, pancreatic cancer, colorectal cancer, renal cancer, liver cancer, brain cancer, lymphoma, leukemia, and lung cancer. In certain aspects, the cancer is a breast cancer. In certain aspects, the cancer is a luminal A breast cancer. In certain aspects, the cancer is a luminal B breast cancer. It should be understood and herein contemplated that luminal A breast cancer refers to breast tumors that are estrogen receptor (ER) positive, progesterone receptor (PR) positive, and HER2 negative. Luminal B breast cancer refers to breast tumors that are estrogen receptor (ER) positive, progesterone receptor (PR) negative, and HER2 positive. “Metastatic breast cancer”, also called stage IV, refers to breast cancer that has spread to another part of the body.

As the timing of a cancer can often not be predicted, it should be understood that the disclosed methods of treating, preventing, reducing, and/or inhibiting a cancer (e.g., luminal B breast cancer or metastatic breast cancer) can be used prior to or following the onset of uncontrolled growth of aberrant cells or metastasis, to treat, prevent, inhibit, and/or mitigate any stage of the cancer. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years;12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of the cancer or a symptom thereof; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of the cancer or a symptom thereof. In some embodiments, the disclosed methods can be employed prior to or following a chemotherapy. In some embodiments, the disclosed methods can be employed prior to or following the administering of another anti-cancer agent. In some embodiments, the disclosed methods further comprise administering to the subject a therapeutically effective amount of another anti-cancer agent.

A MEK inhibitor described herein can be administered to the subject via any route including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. In some embodiments, the MEK inhibitor is administered orally.

Dosing frequency for a MEK inhibitor of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, twice a day, three times a day, four times a day, or five times a day. Administration can also be continuous and adjusted to maintaining a level of the compound within any desired and specified range.

In some embodiments of the methods of treating a cancer, wherein a cancer cell comprises an increased level of the RAD51AP1-DYRK4 gene fusion, an appropriate dosage level of the MEK inhibitor will generally be about 0.01 mg to 40 mg per day, and can be administered in single or multiple doses. In some embodiments, the dosage level is about 0.1 mg to about 10 mg per day. In some embodiments, the dosage level is about 0.1 mg to about 5 mg per day, about 0.1 mg to about 2 mg per day, about 0.1 mg to 2 mg per day, about 0.1 mg to 1 mg per day, or about 0.1 to 0.5 mg per day.

### Kits

Included herein are kits comprising a probe or a set of probes, for example, a detectable probe or a set of amplification primers that specifically recognize a nucleic acid comprising a fusion point or break point. The kit can further include, in the same vessel, or in a separate vessel, a component from an amplification reaction mixture, such as a polymerase, typically not from human origin, dNTPs, and/or UDG. In some embodiments, the amplification primers are selected from the group consisting of SEQ ID NO: 5, SEQ ID NO: 6, SEQ ID NO: 7, SEQ ID NO: 8, SEQ ID NO: 11, SEQ ID NO: 12, SEQ ID NO: 13, SEQ ID NO: 14, SEQ ID NO: 15, SEQ ID NO: 16, SEQ ID NO: 25, and SEQ ID NO: 26. In some embodiments, the amplification primers are selected from the group consisting of SEQ ID NO: 5, SEQ ID NO: 6, SEQ ID NO: 7, SEQ ID NO: 8, SEQ ID NO: 25, and SEQ ID NO: 26. In some embodiments, the detectable probe is selected from polynucleotide sequence that specifically hybridizes to a fusion point nucleotide sequence within SEQ ID NO: 52, SEQ ID NO: 53, or SEQ ID NO: 54. In some embodiments, the kit comprises a detectable moiety that is covalently bonded to the probe. Furthermore, the kit can include a control nucleic acid. For example, the control nucleic acid can include a sequence that includes a fusion point sequence within a sequence selected from the group of SEQ ID NO: 52, SEQ ID NO: 53 and SEQ ID NO: 54.

All patents, patent applications, and publications referenced herein are incorporated by reference in their entirety for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Discovering Chimerical Transcripts Enriched in Luminal B and Metastatic Breast Cancer

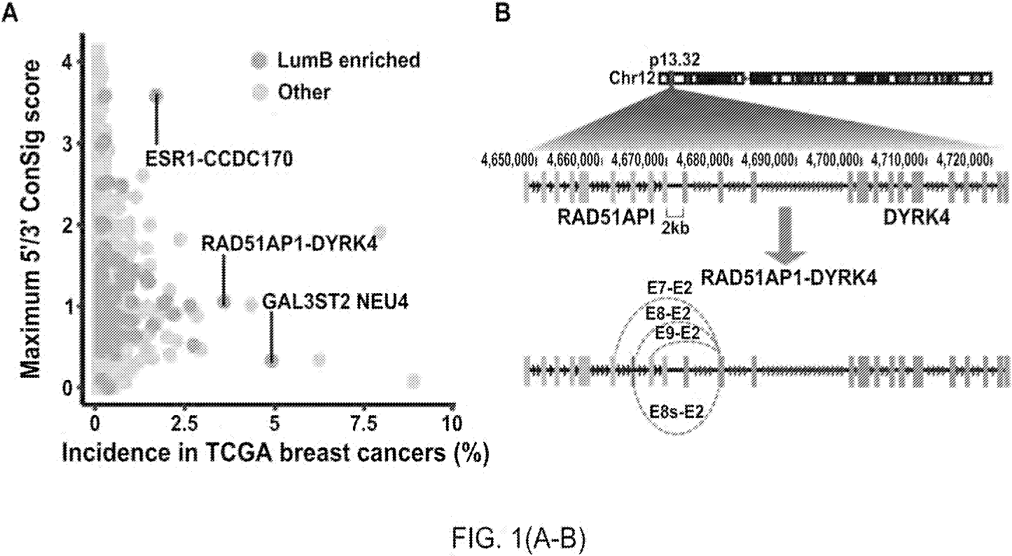

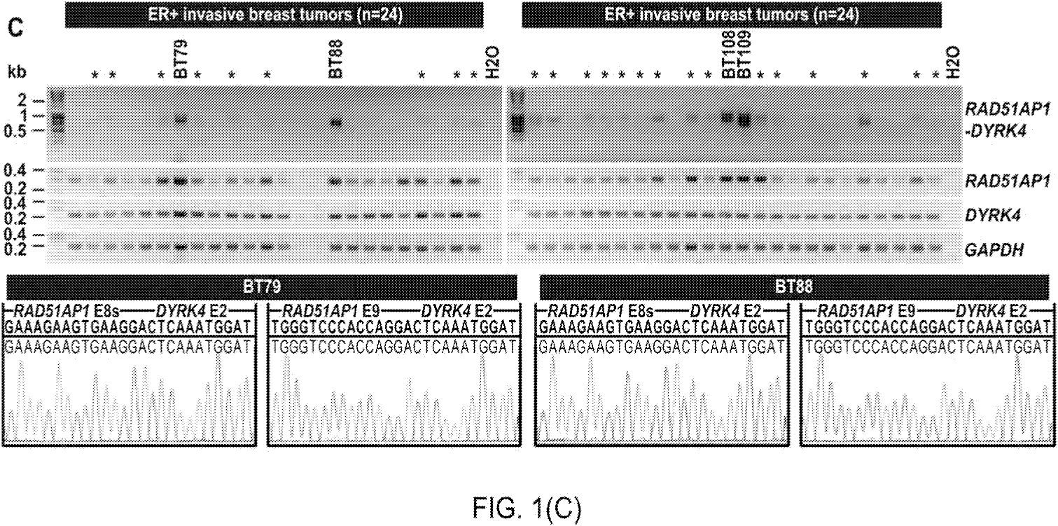

A fusion-zoom pipeline was developed for identifying pathological recurrent gene fusions from RNAseq and copy number datasets (Veeraraghavan, J. et al. (2014)). In this study, to detect tumor-specific fusion transcripts, the RNAseq analysis module of the fusion-zoom pipeline was leveraged to identify the chimerical sequences that are abundantly and frequently present in tumor samples but are not expressed in paired normal breast samples. The paired-end RNAseq data for 1059 breast tumors and 111 paired normal breast tumors were obtained from The Cancer Genome Atlas, and were aligned with the reference genome using parameters allowing for the detection of fusion transcripts between adjacent genes. A total of 1206 somatic recurrent fusion transcripts were identified, and their preferential presence in luminal B tumors versus luminal A tumors was assessed by two- proportion Z-statistics. A total of 90 candidates were found to be enriched in luminal B tumors, which were then ranked by their frequency of detection in breast tumors, and the median number of supporting reads in tumors (FIG. 1a). The fusion candidates were also evaluated by the concept signature (ConSig) score of the partnering genes to prioritize the biologically meaningful fusions (Kim, J.A. et al. (2016); Wang, X.S. et al. (2009)). The ConSig analysis employs molecular concepts characteristic of cancer genes for computationally assessing the biological function of candidate genes in cancer (Wang, X.S. et al. (2009)). Among all chimerical transcripts, the most frequent and abundant chimeras enriched in luminal B tumors were GAL3ST2-NEU4 and RAD51AP1-DYRK4 (Table 1) with RAD51AP1-DYRK4 showing a higher ConSig score. The fusion partners, RAD51 associated protein 1 (RAD51AP1) and Dual-specificity tyrosine-(Y)-phosphorylation regulated kinase 4 (DYRK4), are co-linearly placed neighboring genes located approximately 2kb apart on the same strand of chromosome 12 (FIG. 1b), indicating this fusion as a neoplastic read-through event.

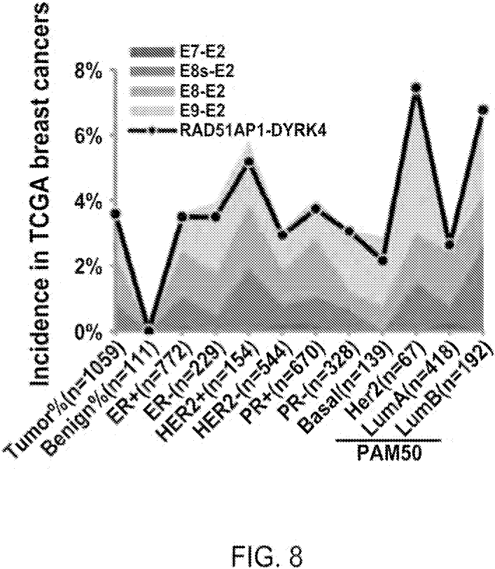

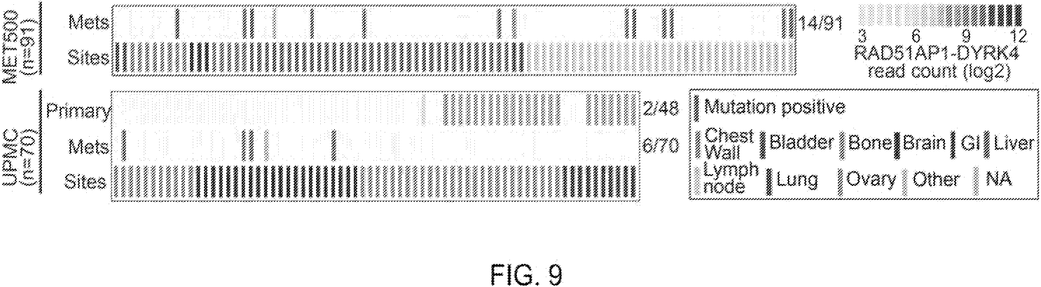

RAD51AP1 is a RAD51-interacting protein specific to the vertebrates. Several studies have shown the involvement of RAD51AP1 in homologous recombination (HR) repair through its interaction with RAD51(Wiese, C. et al. (2007); Dunlop, M.H. et al. (2011). Besides its role in HR repair, enhanced expression of RAD51AP1 has been found to be involved in the growth of intrahepatic cholangiocarcinoma (Obama, K. et al. (2008)). DYRK4 belongs to a conserved family of serine/threonine protein kinases (Park, J., Song, W.J. & Chung, K.C. (2009)); this gene, however, does not contribute any in-frame protein sequences to the fusion protein product. Therefore, it is highly unlikely that the fusion protein acts through DYRK4 kinase activity or serves as dominant negative of DYRK4. Among the 1059 breast tumors sequenced by TCGA, RAD51AP1-DYRK4 chimeric transcript is detected in 38 tumors (3.59 %), and is preferentially present in luminal B tumors (7%) compared to luminal A tumors (3%) (Table 2). RNAseq detected three major fusion variants in the breast tumors and cell lines sequenced by TCGA, namely E9-E2, E8-E2, or E8s-E2 variant transcripts, in which exon 9, 8, or an alternative splicing donor site in exon 8 of RAD51AP1 is fused to exon 2 of DYRK4, respectively (FIG. 1b), with the E9-E2 and E8s-E2 variants more enriched in luminal B tumors (FIG. 8). Further our analysis of RNAseq data for metastatic breast tumors from UM MET500 (Robinson et al. 2017) and UPMC cohorts detected preferential overexpression of RAD51AP1-DYRK4 in 9-15% of metastatic tumors (FIG. 9) compared to 3.6-9.5% of primary tumors, suggesting the enrichment of this fusion in metastatic breast cancers.

### Example 2. Tumor-Specific RAD51AP1-DYRK4 Transcripts Are Ectopically Overexpressed in a Subset of Breast Cancers

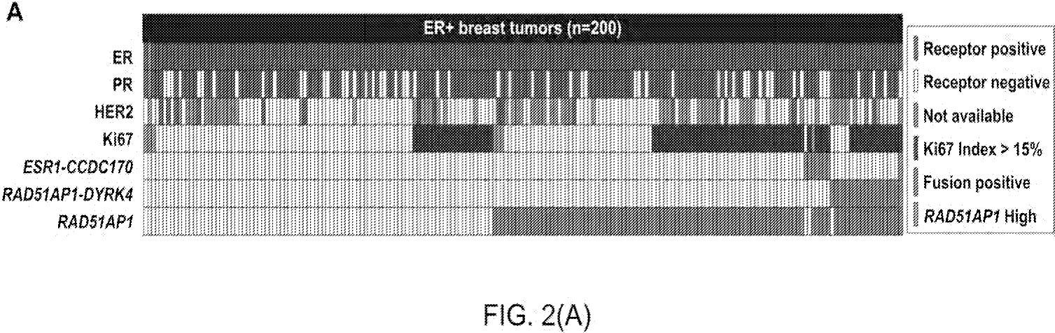

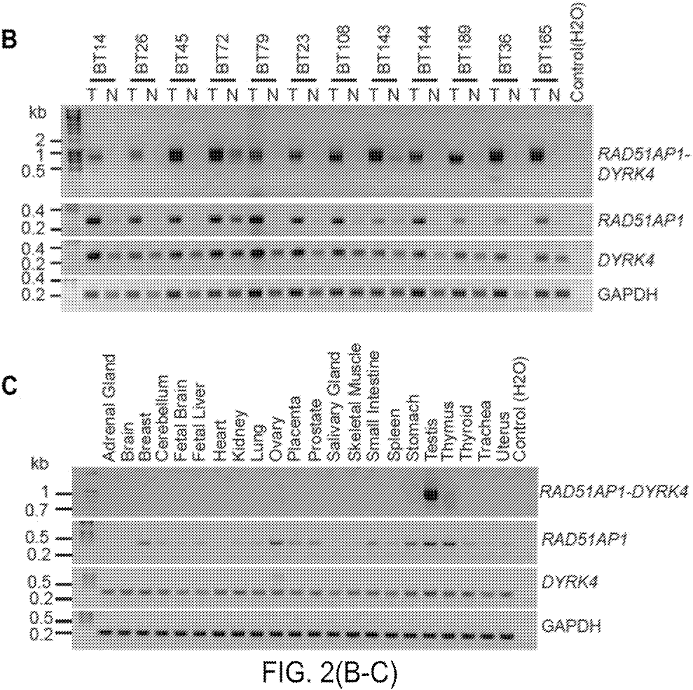

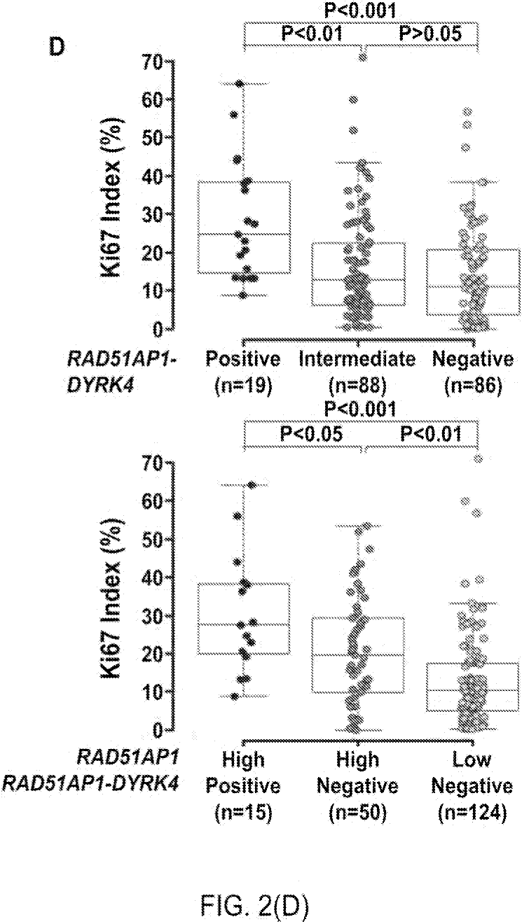

To assess the expression of RAD51AP1-DYRK4 in breast tumor samples, 200 ER+ breast tumor tissues were analyzed by reverse transcription PCR (RT-PCR) using forward primers from Exon 1 of RAD51AP1 and reverse primers from exon 2 of DYRK4 that can detect all of the aforementioned variants. Of the 200 ER+ tumors analyzed, strong RAD51AP1-DYRK4 expression was detected in 19 tumors (9.5%), which was verified by capillary sequencing (FIG. 1c, Table 3). Consistent with the observation in TCGA tumors, in this patient cohort RAD51AP1-DYRK4 expression also tend to be mutually exclusive with ESR1-CCDC170 (FIG. 2a). The fusion transcripts are not detected in the paired adjacent normal breast tissues, indicating their high tumor-specificity (FIG. 2b). To investigate the expression of RAD51AP1-DYRK4 in normal tissues, RT- PCR was performed in 23 types of pooled normal human tissues, including somatic, germ, and fetal tissues. The RAD51AP1-DYRK4 transcript was expressed abundantly in testis, and marginally in thymus, but not in any of the other 21 tissues examined (including breast, ovary, and uterus, FIG. 2c). Such cancer-testis specific expression pattern indicates an important function role of RAD51AP1-DYRK4 in breast cancer (Wang, X. et al. (2018); Watkins, J. et al. (2015); Mahmoud, A.M. (2018)). It is notable that RAD51AP1-DYRK4 expression tends to present in the tumors overexpressing wtRAD51AP1, but not vice versa. This indicates that an active RAD51AP1 promoter may act as a prerequisite for the expression of this fusion, but not all samples with active RAD51AP1 promoter express this chimerical transcript.

Since different oncogene mutations rarely co-exist in the same tumor samples (Sequist, L.V. et al. (2011)), the experiment was for examining if the expression of RAD51AP1-DYRK4 tends to be mutually exclusive with the ESR1-CCDC170 gene fusion previously identified in luminal B tumors. In the 200 ER+ breast tumor tissues analyzed by RT-PCR, strong positivity of ESR1-CCDC170 and RAD51AP1-DYRK4 chimeras also tend to be mutually exclusive (FIG. 2a). Example 3. RAD51AP1-DYRK4 is preferentially overexpressed in luminal B breast tumors.

High Ki67 proliferation index is a biomarker for luminal B tumors, and cutoff of 13-15% positivity is clinically used to differentiate luminal B tumors (Cheang, M.C. et al. (2009); Voduc, K.D. et al. (2010); Tran, B. & Bedard, P.L. (2011)). Ki67 immuno-histochemistry was performed on 193 out of the 200 ER+ tumor tissues that were tested for RAD51AP1-DYRK4 (Veeraraghavan, J. et al. (2014)). The association of RAD51AP1-DYRK4 expression with the Ki67 index was next assessed. In line with the observation from TCGA tumors, the RAD51AP1-DYRK4-positive tumors displayed a significantly higher Ki67 index than the negative cases (p=0.004) (FIG. 2d, upper panel), indicating a significant association of RAD51AP1-DYRK4 with the luminal B subtype. While weak expression of RAD51AP1-DYRK4 was observed in an additional 93 ER⁺ breast tumors (herein termed as intermediate cases), these cases did not demonstrate a significantly increased Ki67 index (p=0.297). Thus, only strong overexpressing cases are considered as fusion-positive in the following studies, which are determined based on RT-PCR band intensities (FIG. 10). Using 15% Ki67 positivity as cutoff, 80 tumors have high Ki67 index, among which 14 cases are fusion- positive (17.5%). Among the 113 Ki67-low tumors, only 5 tumors are fusion-positive (4.4%). Fisher’s exact test indicates a significant enrichment of positive cases in Ki67 high tumors (p=0.006). Next, the Ki67 index was compared between RAD5IAPI-DYRK4 + tumors and the wtRAD51AP1 overexpressing tumors. This revealed a significantly higher Ki67 index in RAD51AP1-DYRK4 + tumors compared to wtRAD51AP 1 overexpressing tumors (p=0.046) (FIG. 2d, lower panel).

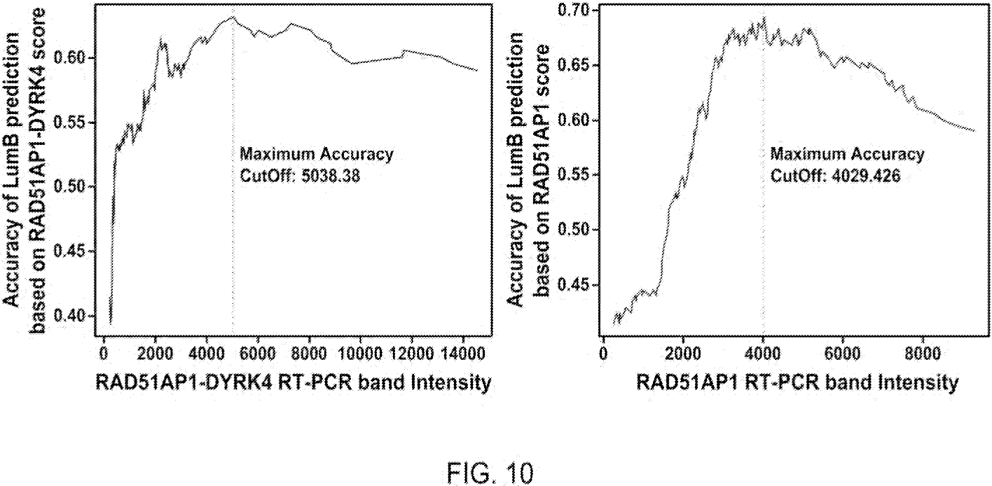

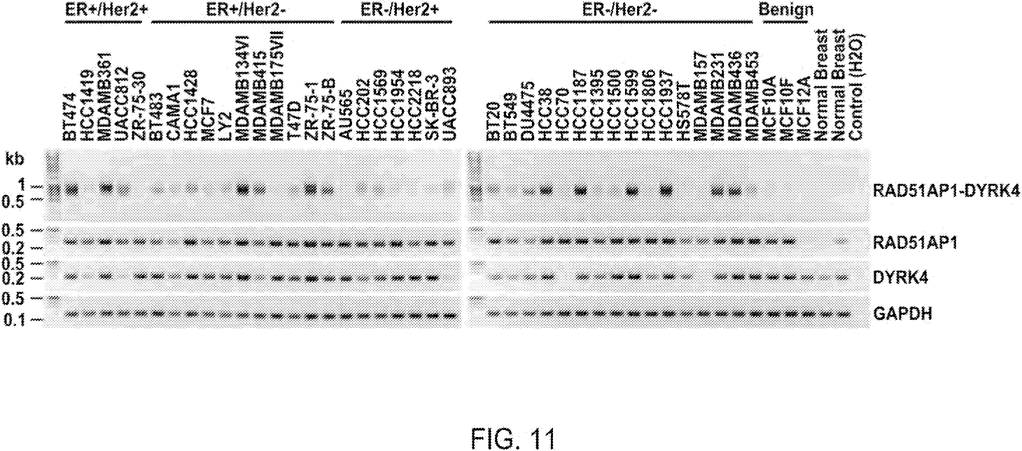

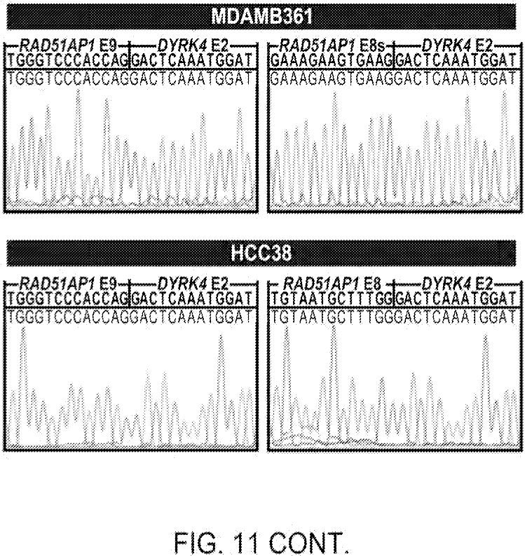

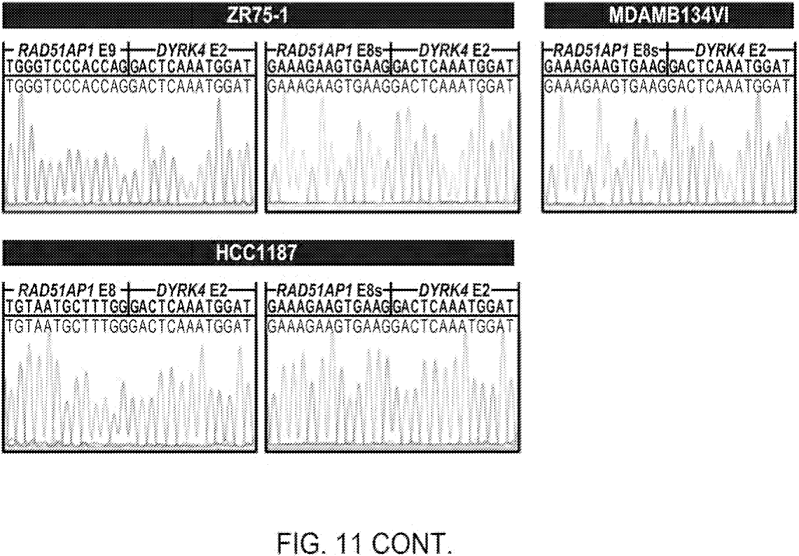

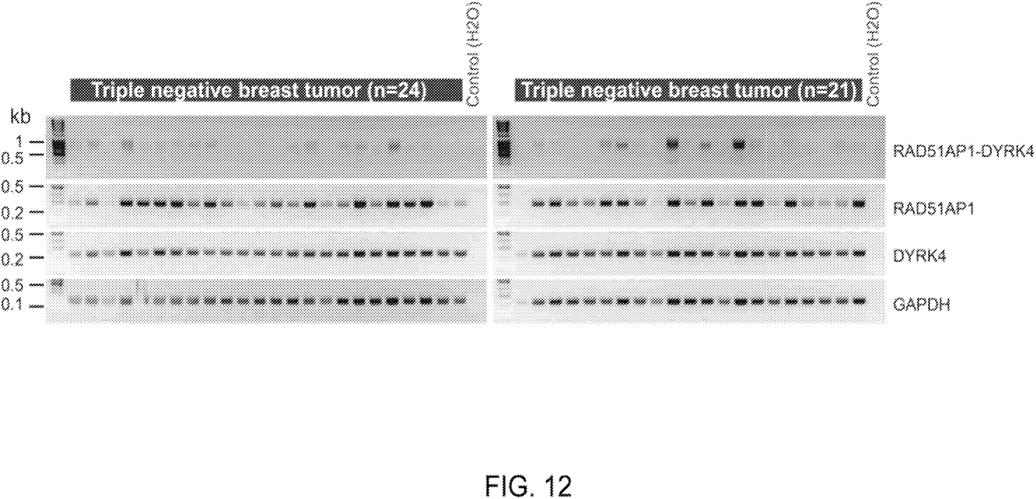

Next, RT-PCR analysis of a panel of breast cancer cell lines was performed, which revealed RAD51AP 1- DYRK4 expression in many cell lines across different breast cancer subtypes, including many triple-negative breast cancer (TNBC) cell lines (FIG. 11). The expression of RAD51AP1- DYRK4 was thus examined in 45 triple-negative breast tumors which revealed only two RAD51AP1-DYRK4 positive cases (FIG. 12). This is consistent with the low RAD51AP1-DYRK4 positivity in TCGA basal-like breast tumors.

### Example 4 Characterization of RAD51AP1-DYRK4 Encoded Protein Products

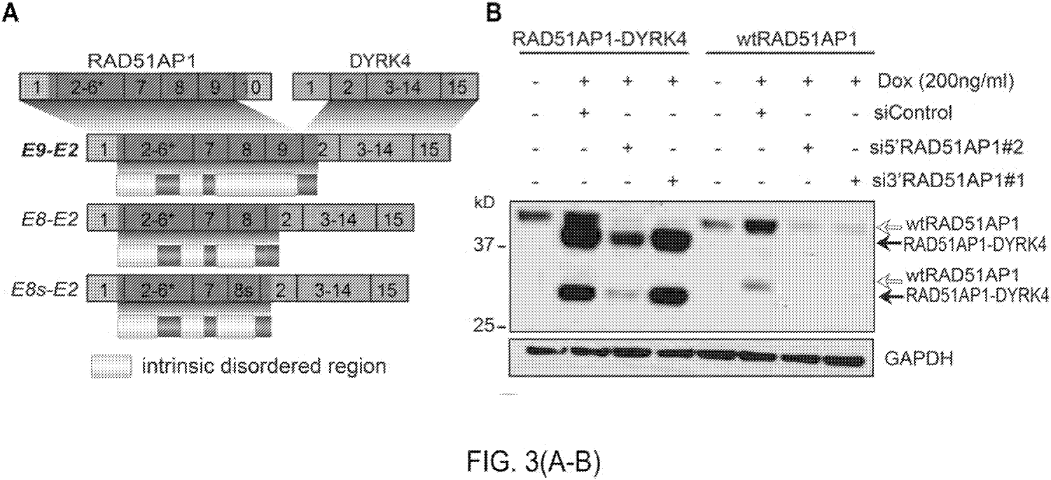

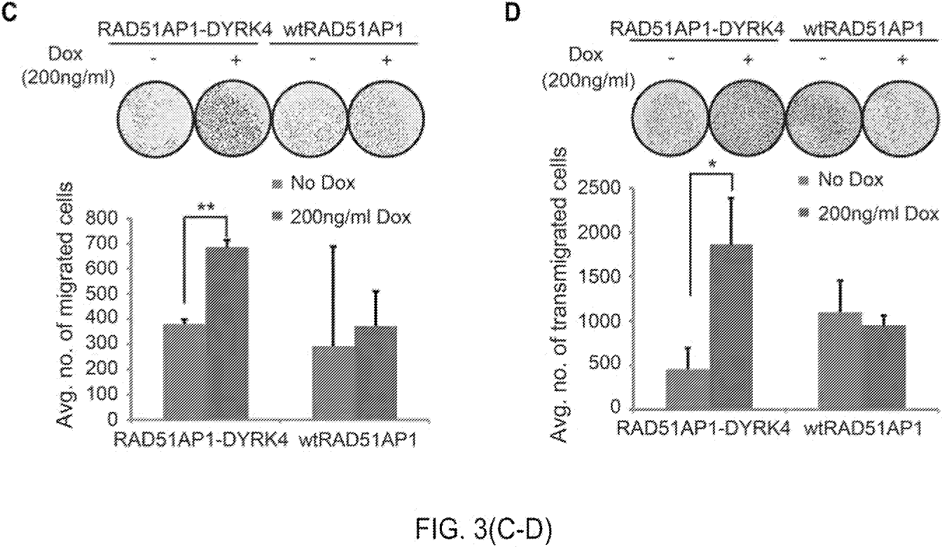

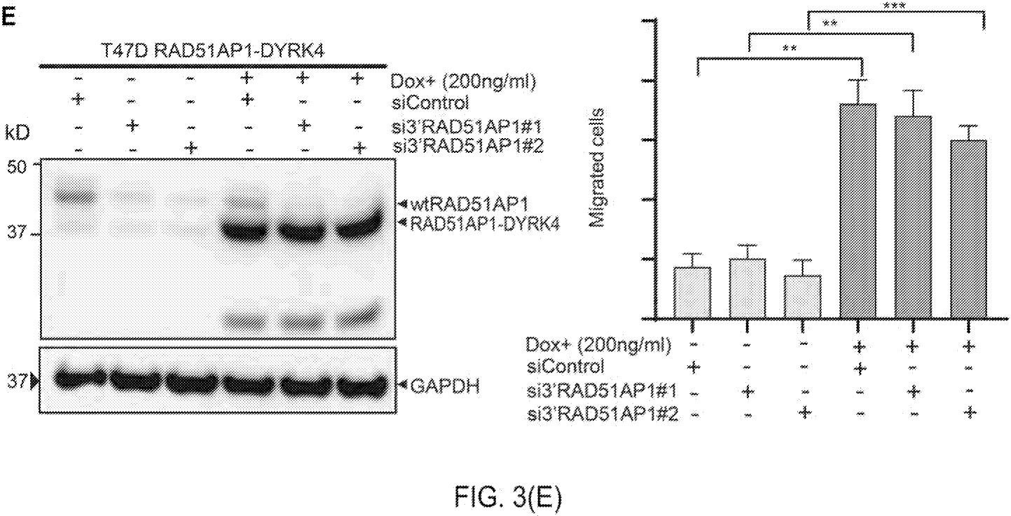

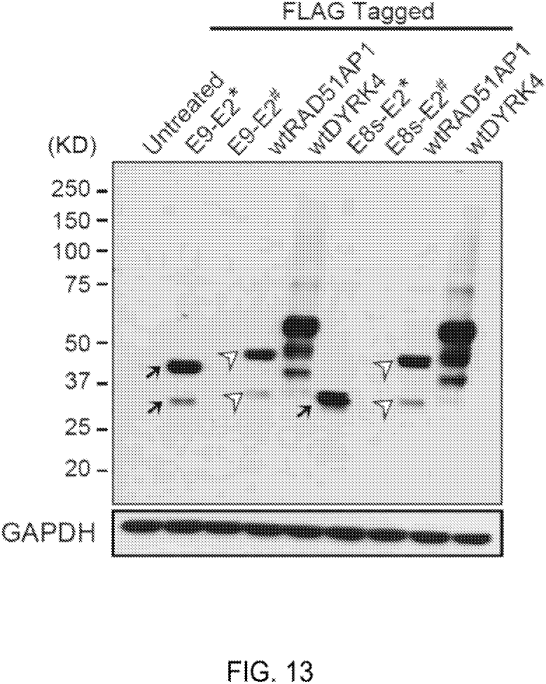

As a common scheme, the RAD51AP1-DYRK4 fusion variants encode a C-terminally truncated RAD51AP1 protein fused to a short fragment of out-of-frame protein sequence from the DYRK4 transcript (FIG. 3a), leading to the loss of RAD51 interacting domain. To test the translatability of RAD51AP1-DYRK4 transcripts in breast cancer, the fusion cDNA was engineered to contain the most common fusion variant E9-E2 chimeric ORF together with the endogenous 5′ translation start sequences into a doxycycline-inducible lentiviral vector, which was then transduced into the T47D luminal-A like breast cancer cells. Western blot analysis using a commercial polyclonal antibody against the N-terminus of RAD51AP1 detected the E9-E2 or wtRAD51AP1 protein bands specific to the transduced T47D cells treated with doxycycline (FIG. 3b). Of note, both E9-E2 and wtRAD51AP1 overexpressing T47D cells exhibited two specific protein bands respectively. To verify the identity of these E9-E2 and wtRAD51AP1 protein bands, we transfected the engineered T47D cells with 5′RAD51AP1 siRNA designed to knockdown both RAD51AP1-DYRK4 and wtRAD51AP1, or the 3′RAD51AP1 siRNA designed to only inhibit the wtRAD51AP1. Subsequent western blots showed that the 5′siRNA but not 3′siRNA silenced both the E9-E2 bands and the wtRAD51AP1 bands induced by doxycycline, which verified the identities of these bands (FIG. 3b). To examine if the DYRK4 coding sequence following the fusion ORF can be translated from the RAD51AP1-DYRK4 transcript, a Flag-tag was added to the 3′ end of the fusion ORF or the 3′ end of the DYRK4 ORF. Immunoblots of T47D cells transfected with these constructs using an anti-Flag antibody detected the fusion protein but not DYRK4 protein (FIG. 13). This suggests that the fusion transcripts do not encode DYRK4 protein.

### Example 5. RAD51AP1-DYRK4 Promotes Cancer Cell Motility and Trans-Endothelial Migration

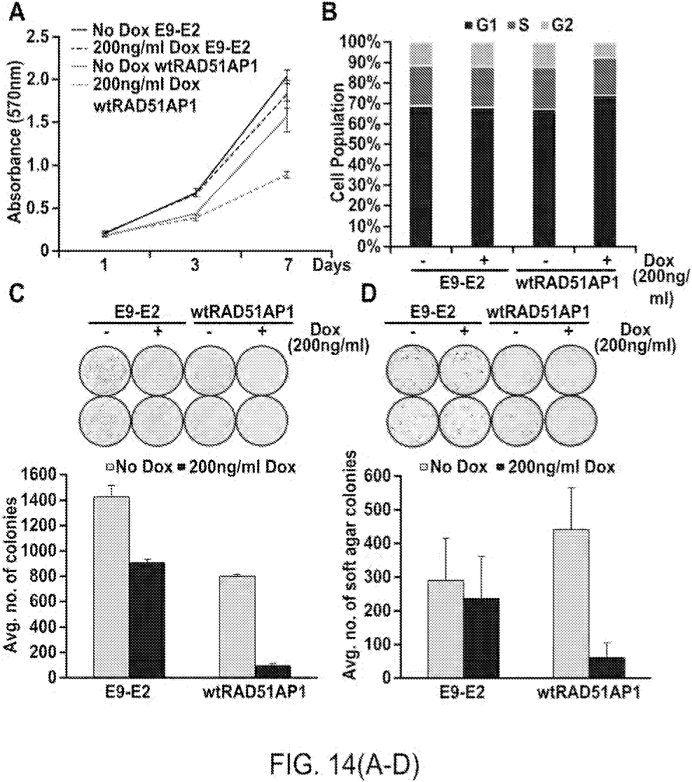

The phenotypic changes were explored in the T47D luminal breast cancer cells inducibly overexpressing E9-E2 or wtRAD51AP1. Transwell migration assays indicated that RAD51AP1-DYRK4 but not wtRAD51AP1 significantly augments the chemotactic migration of T47D breast cancer cells (FIG. 3c). On the other hand, RAD51AP1-DYRK4 did not confer increased cell proliferation or colony-forming capability, whereas wtRAD51AP1 decreased the cell proliferation and colony formation, and increased the G1 cell population (FIG. 14). To mimic the in vivo behavior of tumor cells undergoing extravasation during metastasis (Voura, E.B., et al. (2001)), in vitro transendothelial migration assays were performed to test the effect of RAD51AP1- DYRK4 on trans-endothelial migration of breast cancer cells. The T47D cells inducibly expressing RAD51AP1-DYRK4 or wtRAD51AP1 were allowed to migrate through a confluent monolayer of human umbilical vein endothelial cells (HUVECs). Ectopic expression of RAD51AP1-DYRK4 but not wtRAD51AP1 significantly enhanced the trans-endothelial migration of T47D cells (FIG. 3d). To assess if RAD51AP1- DYRK4 function is dependent on wtRAD51AP1, specific knockdown of wtRAD51AP1 was performed using two siRNAs against its 3′ region not involved in the fusion in the T47D cells inducibly overexpressing RAD51AP1-DYRK4 (FIG. 3e). The results showed that the cell motility is not significantly affected by depletion of wtRAD51AP1 in the presence or absence of exogenous overexpression of the fusion. These data indicate that RAD51AP1-DYRK4 but not wtRAD51AP1 promotes motility and transendothelial migration of luminal breast cancer cells, and the function of the fusion does not depend on the wild-type protein.

### Example 6. Augmented MEK/ERK Signaling is Characteristic of RAD51AP1-DYRK4 Expressing Breast Tumors

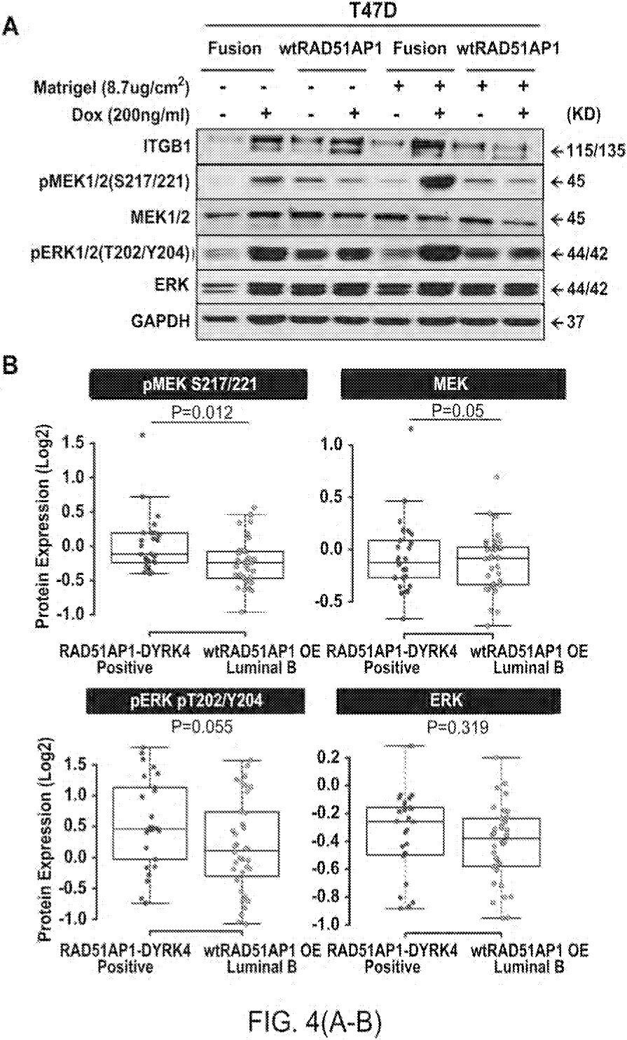

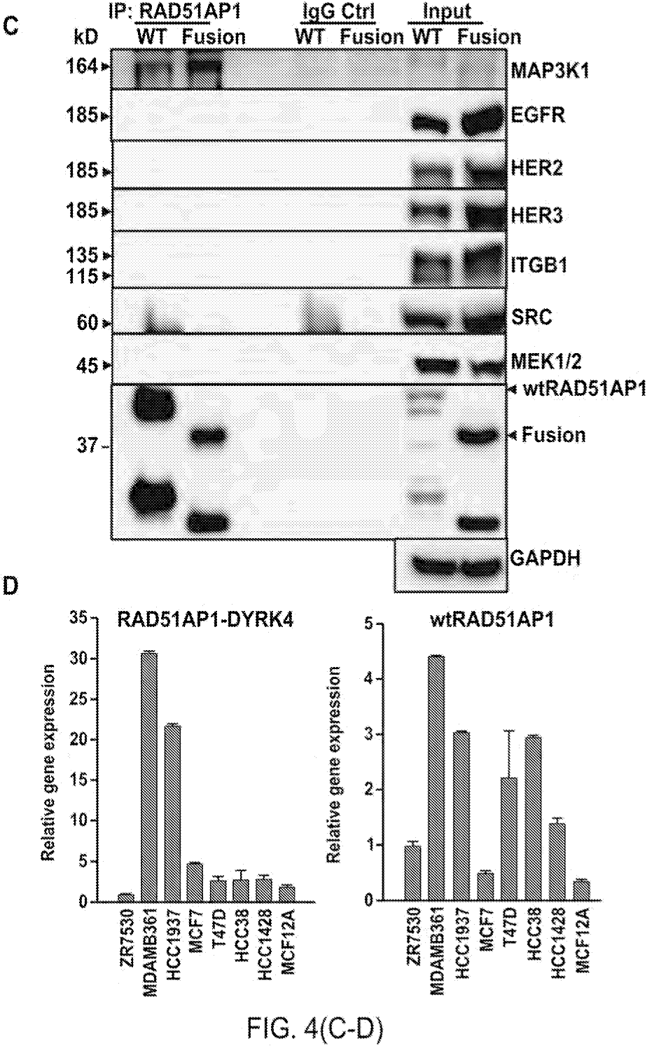

To examine the signaling alterations differentially associated with RAD51AP1-DYRK4 or wtRAD51AP1 expression, immunoblots were performed on the T47D cells ectopically expressing RAD51AP1- DYRK4 or wtRAD51AP1 (FIG. 4a). As a result, substantially increased phosphorylation of MEK/ERK was observed following RAD51AP1-DYRK4 overexpression in T47D cells. Upregulation of integrin B1 (ITGB1) was also observed in fusion-expressing T47D cells. Most of these changes are specific to the RAD51AP1- DYRK4 overexpressing T47D cells, compared to wtRAD51AP1. To explore the impact of extracellular matrix on MEK/ERK signaling associated with RAD51AP1-DYRK4 expression, the signaling alterations were examined in the engineered T47D cells cultured in Matrigel, a solubilized basement membrane preparation rich in ECM proteins (i.e. laminin, collagen IV) (Streuli, C.H. et al. (1995)). With extracellular matrix, the activation of the MEK/ERK cascade were markedly enhanced (FIG. 4a). In addition, this enhancement is highly specific to the T47D cells expressing RAD51AP1-DYRK4-it is not observed in wtRAD51AP1-expressing T47D cells. This indicates that in the breast tumor tissues containing extracellular matrix, RAD51AP1-DYRK4 can play a key role in activating the MEK-ERK signaling. Further, TCGA breast cancer reverse phase protein array (RPPA) data revealed that the fusion-expressing tumors displayed a significantly increased phosphorylation of MEK/ERK, compared to wtRAD51AP1 overexpressing luminal B tumors which support the observations on the T47D ectopic expression model (FIG. 4b).

To identify the key molecules important for RAD51AP1-DYRK4 to modulate MEK signaling, the RAD51AP1 interactants were investigated with the Entrez Gene database. This revealed a RAD51AP1 interactant, MAP3K1, a cytoplasmic protein that regulates ERK, JNK, and p38, and is known to suppress metastasis and induce anoiksis (Pham, T.T., et al. (2013)). Immuno-precipitation was performed using the RAD51AP1 antibody in the T47D cells overexpressing E9-E2 or wtRAD51AP1. This result showed that MAP3K1 protein coprecipitated with both wtRAD51AP1 and E9-E2 proteins, showing their direct functional relations (FIG. 4c). On the other hand, other known MEK upstream signaling proteins such as ErBb receptor kinases, integrin β1, c-Src, or MEK itself did not co-precipitate with RAD51AP1-DYRK4.

### Example 7. Assessing the Function of Endogenous RAD51AP1-DYRK4 Protein in Luminal Breast Cancer Cells

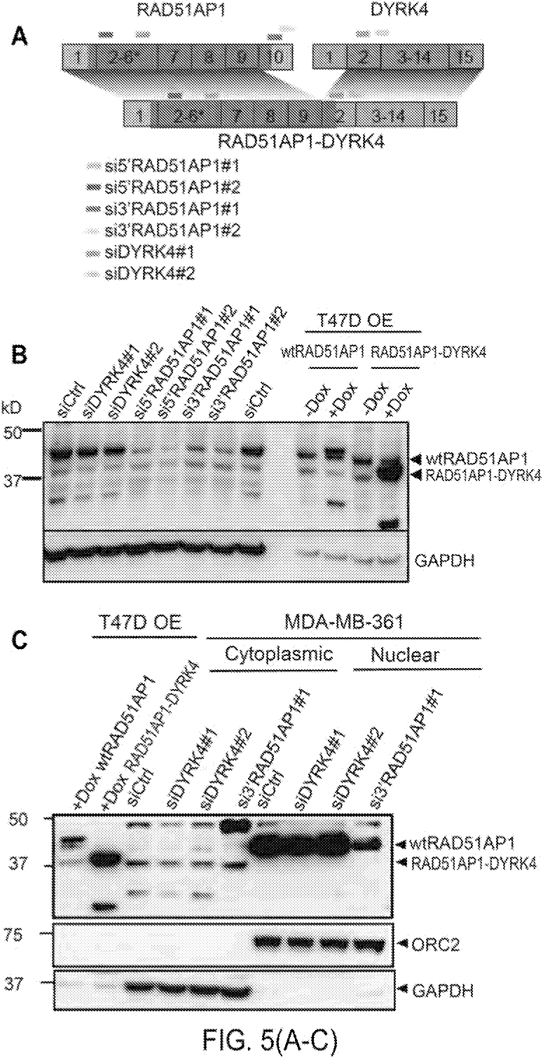

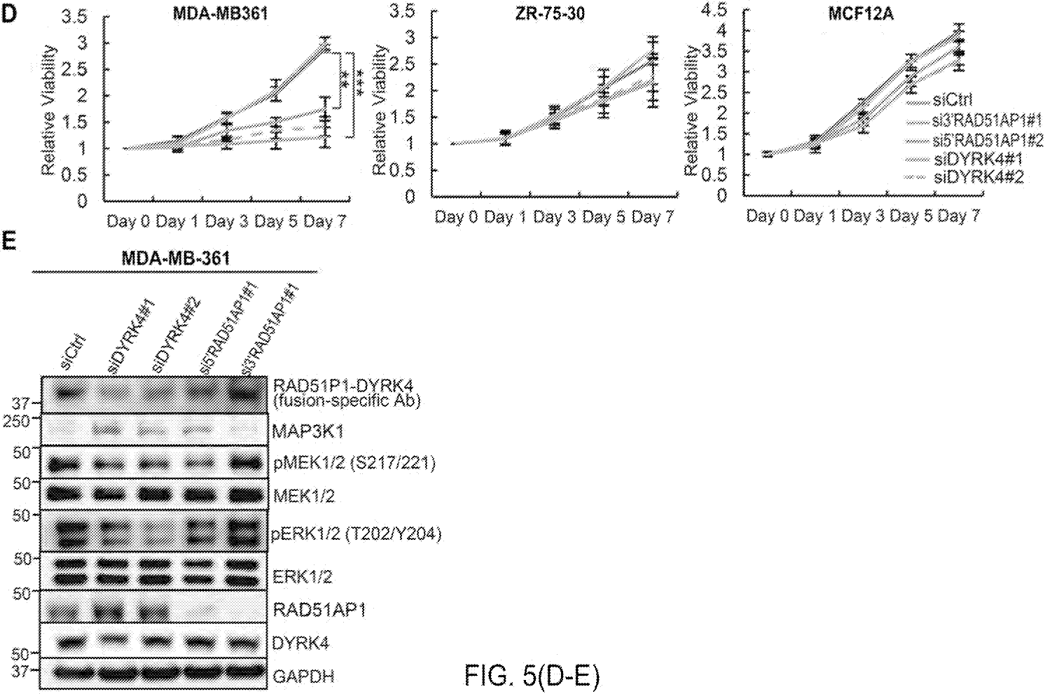

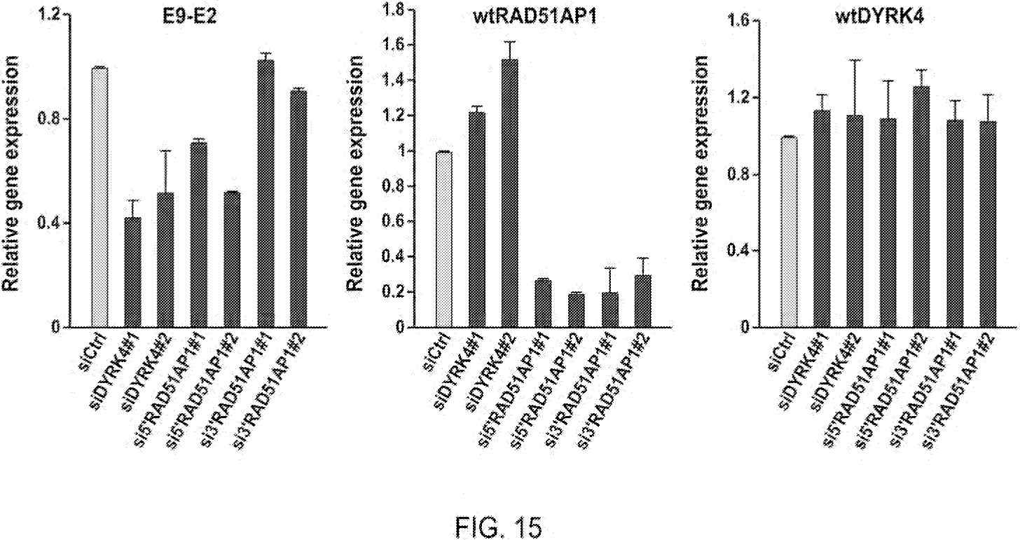

Next, we sought to assess the function of endogenous RAD51AP1-DYRK4 protein overexpressed in MDAMB361 (FIG. 4d). MDAMB361 is an ER+/Her2+ cell line derived from brain metastasis (29) and is resistant to endocrine or her2-targeted therapies (30,31). We thus used this cell line as a model to study the function of the endogenous RAD51AP1-DYRK4. To specifically knockdown RAD51AP1-DYRK4, we designed several siRNAs targeting the fusion junctions, which however, appear to have general toxicity to the cells. We therefore designed two 5′RAD51AP1 siRNAs that knockdown both RAD51AP1-DYRK4 and wtRAD51AP1, and two DYRK4 siRNAs targeting both RAD51AP1-DYRK4 and wtDYRK4, and two 3′RAD51AP1 siRNAs designed to only inhibit the wtRAD51AP1 (FIG. 5A). We then performed Western blot analysis to detect the endogenously expressed RAD51AP1-DYRK4 protein products in the MDA-MB-361 cells. As a result, we were able to readily detect the E9-E2 protein band expressed by the MDAMB361 cells, which can be inhibited by the 5′RAD51AP1 siRNAs and DYRK4 siRNAs, but not by 3′RAD51AP1 siRNAs (FIG. 5B). The levels of the protein inhibitions appear to correlate with the levels of transcript inhibitions by these siRNAs detected by qPCR (FIG. 15).

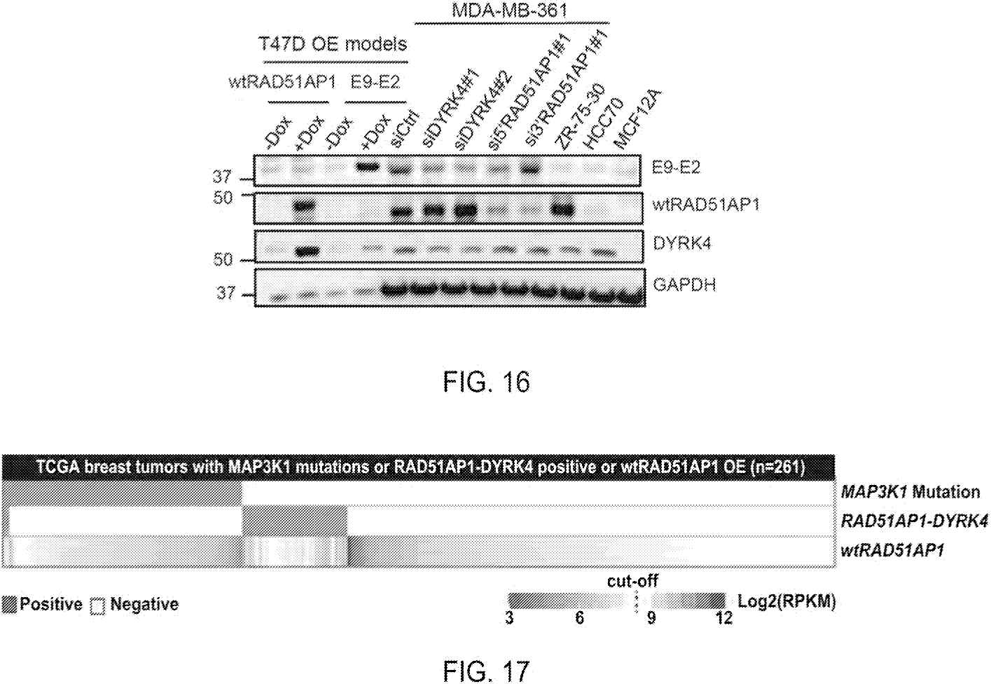

To further verify the identity of the endogenous E9-E2 protein band, we generated a polyclonal antibody against the frameshift DYRK4 peptide, which can specifically detect RAD51AP1-DYRK4 but not wtRAD51AP1. Western blots using this antibody on the MDAMB361 cells detected the previously identified fusion-protein band, which can be inhibited by the siRNAs that can repress the fusion (FIG. 16). This verified the identity of the fusion protein band and further support that the frame-shift peptide derived from DYRK4 instead of the wild-type DYRK4 protein sequence are translated from the DYRK4 portion of the chimerical transcript. We then examined the localization of the endogenous RAD51AP1-DYRK4 in the nuclear or cytoplasmic fractions of MDAMB361 cells. The E9-E2 protein preferentially localizes to cytoplasm, in contrast to the nuclear localization of wtRAD51AP1 (FIG. 5C). This result is consistent with the distinct function of the fusion in modulating cytoplasmic signaling in contrast to the role of wtRAD51AP1 in HR repair.

To further assess the function of the endogenous RAD51AP1-DYRK4 protein, we selected the fusion-positive MDAMB361 cells and the fusion-negative cell line ZR75-30 and MCF12A (FIG. 4D), transfected these cell lines with the selected siRNAs targeting 5′RAD51AP1, 3′ RAD51AP1, or DYRK4, and performed MTS assay (FIG. 5D). The cell proliferation is significantly inhibited by the 5′RAD51AP1 siRNA, and by two DYRK4 siRNAs, but not by the 3′RAD51AP1 siRNA specific to wtRAD51AP1. Such effects are not observed in the negative control cell lines ZR75-30, and MCF12A cells, which verified the specific functional effects of the siRNAs against RAD51AP1-DYRK4. Next, we performed western blots following siRNA treatments to examine the function of the endogenously expressed RAD51AP1-DYRK4 on modulating MEK/ERK signaling in the MDAMB361 model (FIG. 5E). Our result showed that the siRNAs against 5′RAD51AP1 or DYRK4 (targeting RAD51AP1-DYRK4), but not 3′RAD51AP1 (targeting wtRAD51AP1) lead to repression of MEK/ERK signaling. This further support the function of the endogenously expressed RAD51AP1-DYRK4 on regulating MEK/ERK signaling.

### Example 8. RAD51AP1-DYRK4 Endows Increased Sensitivity to MEK Inhibition and Attenuates MEKi Induced PI3K-AKT Activation

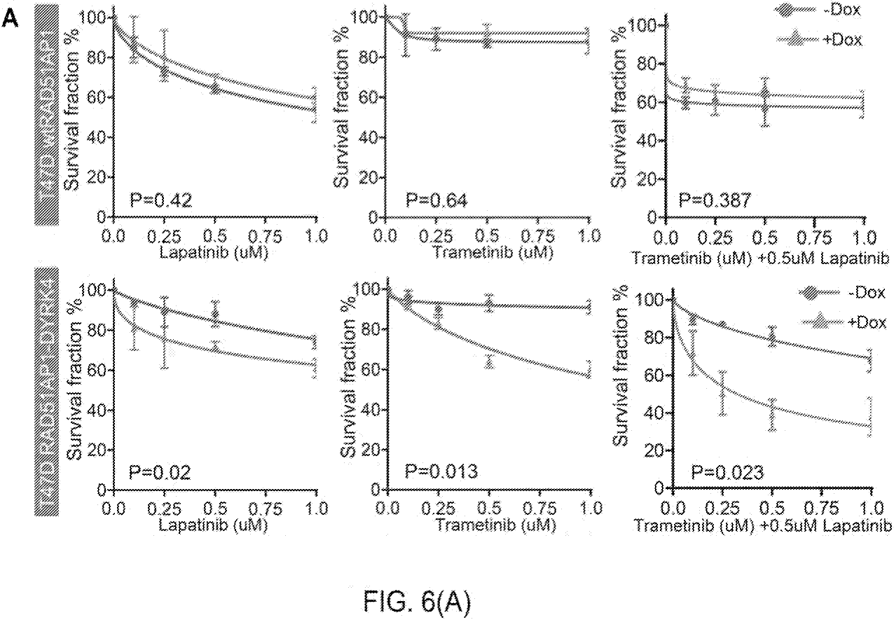

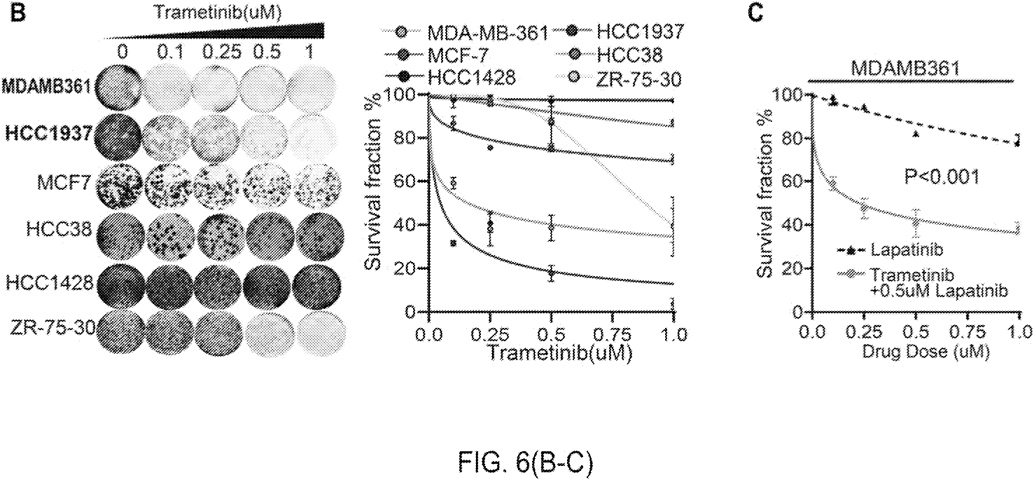

Next, the sensitivity of the engineered T47D cells inducibly expressing RAD51AP1-DYRK4 or wtRAD51AP1 to MEK inhibition was assessed. The first FDA approved MEK inhibitor currently under phase II clinical trial for triple negative breast cancer (NCI 9455) called Trametinib was used for MEK inhibition. MEK inhibition requires longer term drug exposure to exert therapeutic effect (Xue, Z. et al. (2018)). Therefore, clonogenic assays on the T47D models were performed to assess the cell viability following trametinib treatment in the presence or absence of doxycycline induction. Since T47D cells express EGFR, the cells were also treated with lapatinib to observe the combinatory effect. As a result, ectopic expression of RAD51AP1-DYRK4 resulted in significantly increased sensitivity to trametinib, which is not observed following induction of wtRAD51AP1 expression (FIG. 6a). Lapatinib alone or in combination with trametinib did not result in additional therapeutic benefits.

Next, we assessed the trametinib sensitivity in a panel breast cancer cells lines with variable levels of endogenous RAD51AP1-DYRK4 as assessed by real-time PCR (FIG. 4D). As Shown by clonogenic assays, the MDAMB361 and HCC1937 cell lines overexpressing RAD51AP1-DYRK4 showed markedly higher sensitivity to trametinib treatment compared to MCF7, HCC38, HCC1428, and ZR-75-30 cell lines (FIG. 6b). Since MDAMB361 is a HER2 positive cell line we also assessed the therapeutic effect of lapatinib treatment alone or in combination with trametinib. MDAMB361 appeared highly resistant to lapatinib, and the combination treatment yielded similar therapeutic effect as trametinib alone (FIG. 6c). These data suggest that RAD51AP1-DYRK4 endows increased sensitivity to MEK inhibition in the luminal breast cancer cells overexpressing ectopic or endogenous RAD51AP1-DYRK4.

Since inactivating mutations of MAP3K1, which account for about 9% of breast cancer (Koboldt, D.C. et al. (2012); Wee, S. et al. (2009)) has been found to confer increased sensitivity to MEK inhibition30, the mutual exclusivity of RAD51AP1-DYRK4 with MAP3K1 mutation was assessed based on the somatic mutation data for TCGA tumors (FIG. 17). Of the 1059 TCGA tumors analyzed, 81 are MAP3K1 mutation positive and 37 are RAD51AP1-DYRK4 positive, whereas only 2 cases are found to be positive for both, suggesting these as independent events (Fisher’s exact test of dependence: p=1).

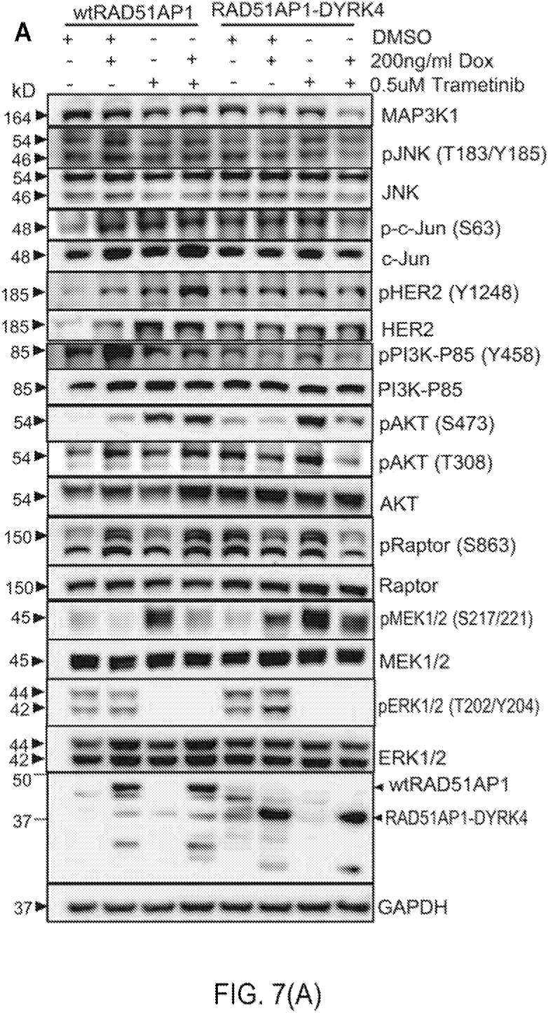

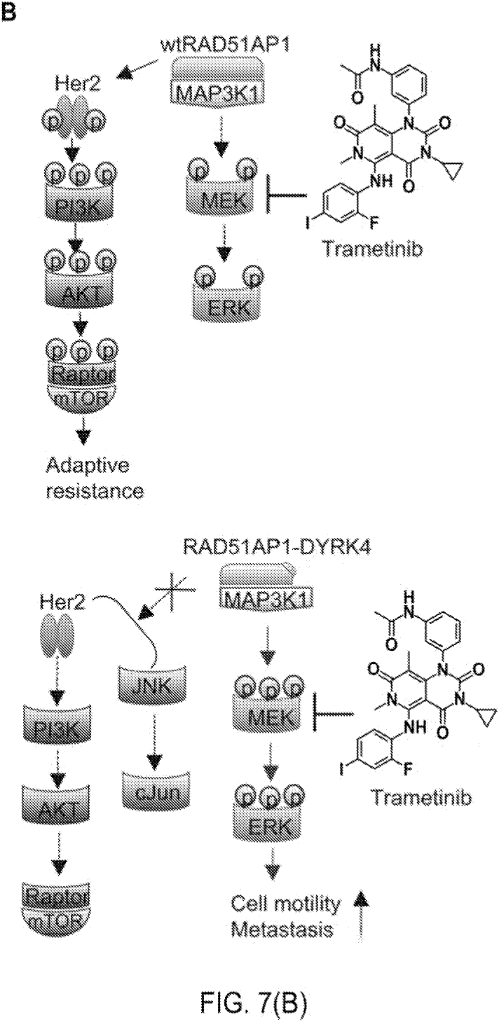

Compensative HER2/PI3K/AKT and MAP3K1/JNK/JUN activation has been reported to mediate resistance to MEK inhibitors (Avivar-Valderas, A. et al. (2018); Maher, C.A. et al. (2009)). We thus examined if RAD51AP1-DYRK4 and wtRAD51AP1 differentially modulate these survival pathways following MEK inhibition. To test this, we treated the engineered T47D cells with 0.5uM of trametinib or vehicle for 24 hours, to assess the early signaling changes following trametinib treatment. Western blot analysis revealed that, under MEK inhibition, RAD51AP1-DYRK4 attenuated HER2/PI3K/AKT/Raptor activation in the T47D cells overexpressing RAD51AP1-DYRK4. In contrast, this compensatory signaling was activated in T47D cells overexpressing wtRAD51AP1 following MEK inhibition (FIG. 7A). In addition, RAD51AP1-DYRK4 also repressed MAP3K1 protein level and JNK-JUN phosphorylation under MEK inhibition. However, we did not observe activation of this signaling following MEK inhibition in the T47D cells ectopically expressing wtRAD51AP1. These results suggest that RAD51AP1-DYRK4 may endow sensitivity to MEK inhibition via repressing compensatory HER2/PI3K/AKT activation (FIG. 7B).

### Example 9 Methods

Analyses of TCGA RNAseq data. The RNAseq (Illumina HiSeq, paired-end) data for breast tumors used in this study were from TCGA cghub (cghub.ucsc.edu). Paired-end RNAseq data from TCGA for 1059 breast tumors and 111 paired normal breast tumors were aligned to human genome build 19 using the Tophat 2.0.3 fusion junction mapper, with parameters allowing for detection of fusion transcripts between adjacent genes (min distance = 5 kb). Using our Perl script pipeline called “Fusion Zoom”, the putative fusion junctions were mapped to human exons (derived from UCSC gene and Ensemble gene) to identify authentic chimerical sequences. The putative fusion transcripts are required to be supported by a minimum of one read that maps to the exon junctions of the two fusion genes. This criterion was expected to filter out most artifactual gene fusions resulting from random ligations during the sequencing library preparation. Putative fusion sequences were then reconstructed and aligned with the human genome and transcriptome using BLAST. The chimeric sequences that can mostly align to a wild-type genomic or transcript sequence were disregarded. The tumor samples that harbor a total of three supporting reads of candidate chimeras are considered as positive cases. After such filtering, the fusion candidates that are found at least two breast tumors with no reads detected in paired adjacent normal breast tissues were identified. A total of 1206 putative fusions were identified as somatic and recurrent; their preferential presence in luminal B tumors compared to luminal A tumors was assessed based on two proportion Z-test with a cutoff of p<0.05. The luminal B enriched fusion candidates were then ranked by the incidence of fusion transcripts in breast tumors, their average abundance (median number of supporting reads), and the concept signature (ConSig) score (consig.cagenome.org, release 2) that prioritizes biologically meaningful candidate genes underlying cancer (Wang, X.S. et al. (2009)).

TCGA RPPA data analysis. Reverse Phase Protein Array (RPPA) data generated based on replicate-based normalization (RBN) was extracted from The Cancer Proteome Atlas (TCPA). The RBN method uses replicate samples run across multiple batches to adjust the data for batch effects (Li, J. et al. (2013)). For analysis, the RPPA results for MEK and ERK signaling in RAD51AP1-DYRK4-positive cases were compared against the fusion-negative luminal B cases overexpressing wtRAD51AP1. Statistical significance was analyzed by Student’s t-test.

Tissue collections. All breast tumor tissues were obtained from the Tumor Bank of the Lester and Sue Smith Breast Center at Baylor College of Medicine. Total RNA for normal breast tissues (5 Donor Pool) was purchased from BioChain (R1234086-P).

RT-PCR. RT-PCR was performed with Platinum Taq Polymerase High Fidelity (Life Technologies) and RAD51AP1-DYRK4 fusion-specific primers (Table 4). RAD51AP1-DYRK4 PCR products from several cell lines and tumors were purified, cloned into pCR4-TOPO vectors, and sequenced. RT-PCR band intensities were quantified using ImageJ software, and the ROCR module of R statistical package was used to determine the optimal cutoff for RAD51AP1-DYRK4 or wtRAD51AP1 overexpression (FIG. 10).

Quantitative real-time PCR. Total RNA was extracted using RAN_{zol} ® RT (Molecular Research Center Inc., Cincinnati, OH, USA) according to the manufacturer’s instructions. RNA was converted to cDNA using the Transcriptor First Strand cDNA Synthesis Kit (Roche). Gene expression level were determined by SYBR Green PCR Master Mix (Applied Biosystems). Analysis was performed using QuantStudio 3 System (ThermoFisher Scientific). The qPCR primers are provided in Table 4. Expression levels were presented relative to the GAPDH (glyceraldehyde-3-phosphate dehydrogenase) housekeeping gene.

Inducible RAD51AP1-DYRK4 expression vector and stable cell lines. RAD51AP1-DYRK4 fusion variants containing the full-length ORFs were amplified from fusion positive cell lines HCC1187 and HCC38, using Roche Expand Long Range dNTPack. The RAD51AP1-DYRK4 fusion cDNAs were then subcloned into an inducible lentiviral pTINDLE vector. After verification by sequencing, these constructs were infected into T47D cells and selected using Geneticin (Invitrogen).

Cell culture. T47D, MDA-MB361, HCC1937, HCC38, HCC1428, MCF12A and human umbilical vein endothelial cells (HUVECs) were obtained from American Type Culture Collection (ATCC). The MCF7 cells were a kind of gift of D. Mark E. Lippman. The ZR-75-30 cells were obtained from NCI-ICBP-45 human breast cancer cell line kit. 293FT cells used for lentivirus packaging were purchased from Invitrogen. T47D, HCC1937, MCF7, HCC38, HCC1428 and ZR75-30 cells were cultured in RPMI 1640 (Cellgro, Corning) with 10% fetal bovine serum, and MDAMB361 cells were cultured in DMEM (Gibco, Thermo Fisher Scientific) with 20% fetal bovine serum (Hyclone, Thermo Fisher Scientific). MCF12A cells were grown in Dulbecco’s Modified Eagle’s/F12 medium (DMEM/F12, 1:1) containing 5% horse serum (Sigma-Aldrich), 20 ng/mL epidermal growth factor, 0.5 µg/mL hydrocortisone, 0.1 µg/mL cholera toxin, and 10 µg/mL human insulin. 293FT cells were cultured in DMEM with 10% fetal bovine serum. HUVECs were cultured using the MEBM basal medium (CC-3151) and MEGM bullet kit (CC-3150) (Lonza).

siRNA knockdown. The 5′RAD51AP1#1 (5′-GCCAGUGAUUAUUUAGAUU-3′) (SEQ ID NO:19), 5′RAD51AP1#2 (5′- GAACAGCACCAAAGGAGUU-3′) (SEQ ID NO:20) and 3′RAD51AP1#1 (5′-CAGAUUAGCACGAGUUAAA-3′) (SEQ ID NO:21), 3′RAD51AP1#2

(5′-CUUCAAGACUUCAAUGAGAUU-3′) (SEQ ID NO:22), DYRK4#1 (5′-CUGCGAAGGUUGGAAGUAAUU -3′) (SEQ ID NO:23) and DYRK4#2 (5′-AUCAAGAACUCCAGAAUGAUU-3′) (SEQ ID NO:24) siRNAs were purchased from Dharmacon and transfected using Lipofectamine RNAi MAX Reagent (Invitrogen) according to manufacturer’s instructions.

Western blot. For immunoblot analysis, E9-E2 and wtRAD51AP1 expression was induced in transduced T47D cells with 200 ng/ml doxycycline for one week. Total proteins were extracted by homogenizing the cells in RIPA Lysis Buffer (Sigma-Aldrich), supplemented with complete protease inhibitor cocktail tablet (Roche Diagnostics), 50 mM beta-Glycerophosphate, 1 mM sodium orthovanadate, 1 mM sodium fluoride, and 1 mM PMSF. Thirty micrograms of protein extracts were denatured in sample buffer, separated by SDS-PAGE, and transferred onto a nitrocellulose membrane (Invitrogen). The membranes were blocked and incubated overnight at 4° C. with primary antibodies. The primary antibodies are provided in Table 5. The membranes were then incubated with the respective horseradish peroxidase-conjugated secondary antibody and the signals were visualized by the enhanced chemiluminescence system (Bio-rad) as per the manufacturer’s instructions. For the blots shown in FIG. 7a, the E9-E2 and wtRAD51AP1 expressing T47D cells were seeded in a 10 cm² dish with or without 200 ng/ml doxycycline treatment and incubated for one week. After doxycycline treatment, the cells were seeded at a density of 1.5×10⁶ in new 6 cm² dishes with or without 200 ng/ml doxycycline containing 0.5uM trametinib or DMSO for 24 hours and harvested cells for immunoblotting analysis.

Immunoprecipitation. The cells were seeded in 10 cm² dishes with 200 ng/ml for one week. After one week doxycycline treatment, doxycycline-induced T47D OE cells were freshly harvested and lysed in NETN-400 buffer (50 nM Tris-HCL, pH 8.0, 400 nM NaCl, 1 mM EDTA, and 0.5% Nonidet P-40) for 25 minutes on ice and then centrifugated for 25 minutes at 14,500 rpm. The supernatants were diluted with the same buffer without NaCl (NETN-0) to obtain a final concentration of NaCl at 150 mM and incubated with indicated antibodies for 2 hours at 4° C., and then added protein-G beads (Santa Cruz) overnight. The beads were washed three times with cell lysis buffer and the precipitated proteins were subjected to western blot analysis.

Subcellular fractionation. Upon siRNA treatment completion, cells were harvested and nuclear and cytoplasmic portions were extracted and separated using the NE-PER® Nuclear and Cytoplasmic Extraction reagents (Thermo Scientific) following the manufacturer’s instructions. Protein concentration were measured by Micro BCA Protein Assay Kit (Thermo Scientific).

Cell proliferation assay. T47D cells expressing E9-E2 or wtRAD51AP1 were seeded at a density of 1000cells/well in a 96-well plate with or without 200 ng/ml doxycycline treatment. The fusion-negative ZR-75- 30 luminal breast cancer and MCF12A benign breast epithelial cell lines were used as negative controls. Cell proliferation was measured by MTS assay at different time points using CellTiter®96Aqueous (Promega) proliferation assay according to manufacturer’s instructions. For the data shown in FIG. 14a, cell proliferation was measured by MTT Cell Proliferation Kit I (Roche) according to manufacturer’s instructions.

Clonogenic assay. The E9-E2 and wtRAD51AP1 expressing T47D cells were seeded at a density of 1000 cells/well in a 6-well plate with or without 200 ng/ml doxycycline treatment and incubated for 14-21 days. The colonies were stained with 0.5% crystal violet in 50% ethanol and counted using GelCount (Oxford Optronix Ltd.). The Trametinib (MEKi) and Lapatinib (EGFR/HER2 inhibitor) used for in vitro therapeutic studies were purchased from Selleck Chemicals. To test their therapeutic effects in the engineered T47D cells and other cell lines, cells (5000-10000, depending on the doubling time) were plated in 24-well for 24 hours prior to treatment with growth media containing trametinib, lapatinib or DMSO was replaced every 4 days for approximately 2 weeks. After this, cells were stained with 0.5% crystal violet in water containing 50% ethanol for 15 minutes at room temperature. The area and intensity of each well was measured using Image J. with Colony Area Plug In.

Soft-agar colony formation assay. The E9-E2 and wtRAD51AP1 expressing T47D cells were suspended in growth medium containing 0.35% SeaPlaque Agarose (Lonza), and plated at a density of 5000 cells/well in a 6- well plate containing 0.7% base agar in growth medium. The cells were then incubated for 21-30 days, and colonies were counted using GelCount.

Migration and transendothelial migration assay. Transwell migration assay and transendothelial migration assay were performed (Veeraraghavan, J. et al. (2014); Cen, J. et al. (2019)). Both of these assays were performed using Boyden chambers (BD Biosciences). The E9-E2 or wtRAD51AP1 expression was induced in transduced T47D cells with or without 200 ng/ml doxycycline for one week. After one-week doxycycline treatment, serum starve the cells overnight. The cells seeded at a density of 2-4×10⁵ in serum-free medium onto 8 µm pore size transwell inserts placed in 24-well plates containing culture medium with 20% FBS. After 48-72 hours, the inserts were removed and stained with hematoxylin. For transendothelial migration assay, HUVECs were seeded in 8 µm transwell inserts and incubated overnight. The serum-starved doxycycline-induced T47D OE cells were seeded on top of confluent HUVEC-coated transwell inserts placed in 24-well containing culture medium with 20% FBS. After 48-72 hours, removed the inserts and the cells were stained as described above. For the data shown in FIG. 3e, the cells were seeded at a density of 4×10⁵ in serum-free medium onto 8 µm pore size transwell inserts placed in 24-well pates containing culture medium with 20% FBS and 30 ng/ml EGF (Sigma-Aldrich). After 48 hour incubation, the inserts were removed and stained with 0.1% crystal violet in 50% methanol for counting using CCD camera associated microscopy (Olympus) and ImageJ.

FACS analysis. For cell cycle analysis, propidium iodide-stained cells were analyzed in a LSRFortessa cell analyzer (BD Biosciences), and cell cycle phases were calculated using FlowJo (flowjo.com).

Statistical analysis. The results of all in vitro experiments were analyzed by student’s t-tests or two-way analysis of variance, and all data are shown as mean ± standard deviation.

TABLE 1

Tumor-specific recurrent fusion candidates that are expressed in >1% of breast tumors and significantly enriched in luminal B tumors compared to luminal A tumors

| Fusion | Type 5′- 3′Placement | 5′- 3′Dist(kb) 5′ConSig | 3′ConSig 5′+3′ConSig | Tumor% (n=1059) | AdjN% (n=111) | Median p-value Reads (n) (lumB:A) |
|---|---|---|---|---|---|---|
| GAL3ST2-NEU4 | adjacent >> | 12 0.22 | 0.33 0.55 | 0.0491 | 0 | 461.5 0.046 |
| RAD51AP1-DYRK4 | neighbor >> | 53 1.05 | 0.80 1.85 | 0.0359 | 0 | 387 0.007 |
| UBE2J2-FAM132A | neighbor >> | 8 0.52 | 0.36 0.88 | 0.0274 | 0 | 178 0.015 |
| ARHGAPIIA-SCG5 | neighbor >> | 48 1.01 | 0.77 1.78 | 0.0264 | 0 | 1466 0.027 |
| IFI6-FRMD5 | interchr - | -0.91 | 0.51 1.42 | 0.0217 | 0 | 5 0.001 |
| LINC00176-TCEA2 | adjacent >> | 30 0.00 | 1.07 1.07 | 0.0198 | 0 | 5 0.000 |
| METTL8-RERG | interchr - | -0.43 | 1.04 1.47 | 0.0189 | 0 | 5.5 0.006 |
| ESRI-CCDC170 | neighbor << | 329 3.58 | 0.58 4.16 | 0.017 | 0 | 12.5 0.008 |
| PRRII-SMG8 | neighbor >> | 14 0.77 | 0.30 1.07 | 0.0161 | 0 | 1735 0.011 |
| KIAA0101-CSNKlGl | neighbor >> | 9 1.11 | 1.30 2.41 | 0.0151 | 0 | 3.5 0.004 |
| P2RY6-ARHGEF17 | neighbor >> | 67 0.67 | 1.36 2.03 | 0.0142 | 0 | 5 0.011 |
| WDR35-TTC32 | neighbor >> | 8 0.64 | 0.17 0.81 | 0.0113 | 0 | 5.5 0.029 |
| HDAC6-ERAS | adjacent >> | 4 1.39 | 0.78 2.17 | 0.0104 | 0 | 2349 0.001 |
| HNRNPHI1-CANX | adjacent <> | 92 1.24 | 1.45 2.69 | 0.0104 | 0 | 4 0.011 |
| N4BP3-RMND5B | neighbor >> | 20 0.55 | 0.82 1.38 | 0.0104 | 0 | 6 0.025 |
| CPSF6-C9orf3 | interchr - | -1.06 | 0.79 1.84 | 0.0104 | 0 | 4 0.030 |

Note: >>, 5′ and 3′ genes locate at the same strand with 5′ gene placed upstream of 3′ gene; <<5′ and 3′ genes locate at the same strand but 3′ gene is placed upstream of 5′ gene; <> 5′ and 3′ genes are in different strand. The p values are calculated based on z-test comparing the difference between proportions.
*These two ESR1-CCDC170+ cases are marginal cases supported by two fusion reads. AdjN, paired adjacent normal breast tissues. Dist, Distance.

TABLE 2

The clinical information of RAD51AP1-DYRK4 and/or ESR1-CCDC170 positive cases based on the TCGA RNAseq dataset. IND, Indeterminate. NA, Not Available

| TCGA Patient ID | Age | Gender | Menopause | RAD51AP1-DYRK4reads(n) | ESR1-CCDC170 status | PAM50(RNAseq | Histology | ER(IHC) | PR(IHC) | Her(IHC) | T | N | M | AJCC Stage |
|---|---|---|---|---|---|---|---|---|---|---|---|---|---|---|
| TCGA-A1-A0SO | 67 | F | Post | 2452 | | Basal | Ductal | - | - | Equivocal | T2 | N1 | M0 | IIB |
| TCGA-AR-A24H | 65 | F | Post | 1796 | | LumB | Lobular | + | + | - | T2 | N0 | M0 | IIA |
| TCGA-D8-A13Y | 52 | F | Post | 1562 | | LumB | Ductal | + | + | - | T1 c | N0 | M0 | IA |
| TCGA-BH-A204 | 80 | F | Post | 1522 | | LumB | Ductal | NA | NA | NA | T2 | N1 b | M0 | IIB |
| TCGA-A7-A26J | 49 | F | Pre | 1501 | | LumA | Ductal | + | + | - | T2 | N0 | M0 | IIA |
| TCGA-E9-AIN9 | 58 | F | Post | 1493 | | Basal | Mixed | - | + | + | T2 | N0 | M0 | IIA |
| TCGA-A8-A07L | 58 | F | Post | 1291 | | LumB | Ductal | + | + | - | T3 | N1a | M0 | III A |
| TCGA-E9-A1RB | 40 | F | Pre | 1100 | | LumB | Ductal | NA | NA | NA | T2 | N0 | M0 | IIA |
| TCGA-E2-A15S | 34 | F | Post | 1073 | | LumB | Ductal | + | - | Equivocal | T2 | N1 | M0 | IIB |
| TCGA-C8-A27A | 48 | F | Peri | 974 | | LumB | Ductal | + | + | - | T2 | N1 | M0 | IIB |
| TCGA-B6-A0RU | 49 | F | IND | 643 | | Basal | Other | - | - | NA | T1 c | N0 (i-) | M0 | IA |
| TCGA-AR-A24R | 45 | F | Pre | 585 | + | LumB | Ductal | + | + | - | T1 | N2 | M0 | III A |
| TCGA-AR-A1AW | 65 | F | Post | 505 | | LumB | Ductal | + | + | Equivocal | T2 | N0 | M0 | IIA |
| TCGA-A2-A0YG | 63 | F | Post | 504 | + | LumB | Ductal | + | + | + | T2 | N3a | M0 | IIIC |
| TCGA-C8-A26Y | 90 | F | Post | 489 | | Her2 | Ductal | - | - | - | T2 | N0 | M0 | IIA |
| TCGA-A8-A08L | 89 | F | Post | 455 | | Her2 | Ductal | + | - | - | T3 | N2a | M0 | III A |
| TCGA-Al-AOSN | 50 | F | Post | 442 | + | LumB | Ductal | + | + | + | T1 c | N1 | M X | IIA |
| TCGA-JL-A3YX | 46 | F | Post | 437 | | | Lobular | + | + | + | T2 | N0 | M0 | IIA |
| TCGA-E2-A15L | 65 | F | Post | 393 | | LumA | Lobular | + | + | Equivocal | T2 | N0 | M0 | IIA |
| TCGA-A2-A0ET | 58 | F | Post | 381 | | LumA | Ductal | + | + | Equivocal | T2 | N2a | M0 | III A |
| TCGA-A2-A04V | 39 | F | Pre | 381 | | LumA | Ductal | + | + | Equivocal | T2 | N0 (i-) | M0 | IIA |
| TCGA- A7-A4SF | 54 | F | Post | 364 | | | Ductal | + | - | + | T2 | N0 | M0 | IIA |
| A2-A0D1 | 76 | F | Post | 362 | Her2 | | Ductal | - | - | + | T2 | N0 (i) | M0 | IIA |
| TCGA- E9-A1NI | 51 | F | Post | 336 | LumB | | Mixed | + | + | - | T2 | N0 | M0 | IIA |
| TCGA- A2-A0T1 | 55 | F | Post | 312 | Her2 | | Ductal | - | - | + | T3 | N3 | M0 | IIIC |
| TCGA- E2-A14P | 79 | F | Post | 279 | Her2 | | Ductal | - | - | + | T2 | N3 | M0 | IIIC |
| E2-A1IH | 80 | F | Post | 257 | LumA | | Lobular | + | + | Equivocal | T1 | N0 | M0 | I |
| OL-A66J | 80 | F | Post | 247 | | | Lobular | + | + | NA | T1 | N0 | M X | I |
| TCGA- A8-A09R | 82 | F | Post | 247 | LumB | | Ductal | + | + | - | T2 | N1 | M0 | IIB |
| BH-A1FB | 60 | F | Post | 235 | LumA | | Ductal | + | + | - | T2 | N1 | M0 | IIB |
| TCGA-GM- A3NW | 63 | F | Post | 225 | | | Lobular | - | + | Equivocal | T2 | N0 (i-) | M0 | IIA |
| C8-A1HI | 40 | F | Pre | 195 | LumA | | Ductal | + | + | - | T2 | N2 | M0 | III |

TABLE 3

Clinical information for the ER-positive breast tumor samples analyzed by reverse transcription PCR in this study

| Sample ID | Age | Sex | Race | RAD51AP1-DYRK4 (RT-PCR brand Intensity) | RAD51AP1-DYRK4 Positivity | RAD51AP1(RT-PCR brand Intensity | RAD51AP1 Overexpression | ESRI-CCDC170 Status | MP Stat | ER | PR | HER-2 | Grade | AJCC-TNM | AJCC Stage | Ki67 Score (%) |
|---|---|---|---|---|---|---|---|---|---|---|---|---|---|---|---|---|
| BT14 | 53 | F | Caucasian | 5038.376 | + | 9288.912 | + | - | Post | + | + | - | 1 | T4N2M0 | IIIB | 56 |
| BT36 | 62 | F | Caucasian | 6008.861 | + | 8800.255 | + | + | Post | + | - | NA | 1 | T2N2M0 | IIIA | 64.07 |
| BT79 | 47 | F | Caucasian | 8273.276 | + | 8464.912 | + | Weak | NA | + | + | - | 1 | T1N1M0 | IIA | 44 |
| BT109 | 55 | F | Caucasian | 13605.74 | + | 7845.912 | + | - | Post | + | - | - | 1 | T2NXMX | IIA | 28.22 |
| BT72 | 44 | F | Caucasian | 14533.426 | + | 7561.912 | + | Weak | Pre | + | - | + | 1 | T2N2M0 | IIIA | 20.63 |
| BT108 | 46 | F | Caucasian | 9249.447 | + | 7252.033 | + | - | Pre | + | + | - | 1 | T3N1M0 | IIIA | 36.28 |
| BT165 | 58 | F | Caucasian | 7089.447 | + | 7156.326 | + | Weak | Post | + | + | NA | 1 | T1cN1M0 | IIB | 8.75 |
| BT23 | 58 | F | Caucasian | 8040.154 | + | 6647.397 | + | Weak | Post | + | + | - | 1 | T2N1M0 | IIB | 23 |
| BT45 | 61 | F | Caucasian | 8888.933 | + | 6460.619 | + | - | Post | + | + | - | 1 | T1cNXM0 | I | 38 |
| BT144 | 59 | F | Caucasian | 9664.104 | + | 6422.376 | + | - | Post | + | + | NA | 1 | T2NXM0 | IIA | 13.37 |
| BT88 | 47 | F | Caucasian | 13123.125 | + | 6082.619 | + | - | Pre | + | + | NA | 1 | T2NOM0 | IIA | 27.48 |
| BT35 | 52 | F | Caucasian | 11674.518 | + | 5472.497 | + | - | Pre | + | - | - | 1 | T2N2M0 | IIIA | 38.74 |
| BT132 | 54 | F | Caucasian | 5714.225 | + | 4844.912 | + | - | Post | + | + | NA | 2 | T1N1M0 | IIA | 24.69 |
| BT130 | NA | F | Caucasian | 6506.811 | + | 4756.205 | + | - | NA | + | + | NA | 1 | T1N2M0 | IIIA | 13.17 |
| BT143 | 68 | F | Caucasian | 8834.69 | + | 4089.79 | + | - | Post | + | + | - | 3 | T2NXM0 | IIA | 19.21 |
| BT189 | 48 | F | Caucasian | 11634.439 | + | 3860.083 | - | - | Pre | + | + | NA | 1 | T2N1M0 | IIB | 13.18 |
| BT57 | 43 | F | Caucasian | 7276.276 | + | 3660.74 | - | - | Pre | + | + | NA | 1 | T2N2M0 | IIIA | 15.68 |
| BT26 | 59 | F | Caucasian | 5217.569 | + | 3531.305 | - | - | Post | + | + | - | 1 | T2N1M0 | IIB | 44.56 |
| BT70 | 53 | F | Caucasian | 5838.983 | + | 2867.619 | - | - | Post | + | + | NA | 1 | T2N3M0 | IIIC | 13.15 |
| BT160 | 58 | F | Caucasian | 2470.79 | Weak | 8293.669 | + | Weak | Post | + | + | - | 1 | T1NOM0 | I | 32.25 |
| BT113 | 65 | F | Caucasian | 1578.426 | Weak | 7792.083 | + | + | Post | + | - | - | 1 | T1cN2M0 | IIIA | 36.13 |
| BT52 | 58 | F | Caucasian | 917.749 | Weak | 7638.154 | + | - | Post | + | + | - | 1 | T1cNXM0 | I | 2.92 |
| BT137 | 58 | F | Caucasian | 1717.841 | Weak | 7504.912 | + | Weak | Post | + | - | NA | 1 | T1cNXM0 | I | 51.92 |
| BT135 | 57 | F | Caucasian | 1941.598 | Weak | 7504.669 | + | Weak | Post | + | + | - | 1 | T2NXM0 | IIA | 43.43 |
| BT110 | 67 | F | Caucasian | 3819.79 | Weak | 7299.033 | + | Weak | Post | + | + | - | 1 | T2N0M0 | IIA | 0.39 |
| BT164 | 51 | F | Caucasian | 2755.326 | Weak | 7221.033 | + | - | NA | + | + | NA | 1 | T3N0MX | IIB | 19.75 |
| BT181 | 81 | F | Caucasian | 2452.719 | Weak | 7043.205 | + | Weak | Post | + | + | NA | 1 | T2N0M | IIA | 15.5 |
| BT44 | 71 | F | Caucasian | 2223.548 | Weak | 7041.326 | + | - | Post | + | - | - | 1 | T2NXM0 | IIA | 41 |
| BT106 | 32 | F | Caucasian | 3017.376 | Weak | 6696.619 | + | - | Pre | + | - | + | 1 | T2NXM0 | IIA | |
| BT59 | 53 | F | Caucasian | 3310.719 | Weak | 6626.033 | + | - | Pre | + | + | - | 1 | T2N1M0 | IIB | 3.29 |
| BT24 | 74 | F | Caucasian | 1985.012 | Weak | 6014.74 | + | Weak | Post | + | + | - | 1 | T2NXMX | IIA | 14.34 |
| BT21 | 69 | F | Caucasian | 1531.477 | Weak | 5861.69 | + | Weak | Post | + | - | - | 3 | T4N1M0 | IIIB | 2.67 |
| BT195 | 60 | F | Asian | 4520.004 | Weak | 5783.912 | + | Weak | Post | + | + | NA | 1 | T3N1M0 | IIIA | 2.47 |
| BT139 | 60 | F | Caucasian | 1128.184 | Weak | 5691.79 | + | - | Post | + | + | NA | 1 | T1NXM0 | I | 36.59 |
| BT159 | 41 | F | Caucasian | 2471.083 | Weak | 5438.79 | + | - | Pre | + | - | - | 1 | T2N1MX | IIB | 0.79 |
| BT184 | 58 | F | Caucasian | 2224.134 | Weak | 5324.619 | + | - | Post | + | + | NA | 1 | T2NXM0 | IIA | 17.16 |
| BT95 | 41 | F | Caucasian | 2492.305 | Weak | 5235.376 | + | Weak | Pre | + | + | NA | 1 | T2N1M0 | IIB | 26.34 |
| BT194 | 59 | F | Caucasian | 757.79 | Weak | 5173.548 | + | Weak | Post | + | + | - | 1 | T2N0M0 | IIA | 20.56 |
| BT78 | 65 | F | Caucasian | 2733.134 | Weak | 5156.276 | + | - | Post | + | + | - | 2 | T1bN0M0 | I | 8.46 |
| BT198 | 85 | F | Caucasian | 3963.154 | Weak | 5129.205 | + | - | Post | + | + | - | 1 | T2N2M0 | IIIA | 27.28 |
| BT33 | 36 | F | Caucasian | 1560.113 | Weak | 5128.376 | + | - | Pre | + | - | - | 1 | T1NXM0 | I | 6.12 |
| BT56 | 52 | F | Caucasian | 3790.569 | Weak | 5012.861 | + | - | Post | + | - | - | 2 | T1N1M0 | IIA | 42 |
| BT104 | 66 | F | Caucasian | 3019.205 | Weak | 4978.326 | + | - | Post | + | - | - | 2 | T2N0M0 | IIA | 11.38 |
| BT151 | 56 | F | Caucasian | 3159.79 | Weak | 4945.912 | + | - | Post | + | + | NA | 1 | T2NXM0 | IIA | 7.73 |
| BT37 | NA | F | Caucasian | 1304.903 | Weak | 4928.79 | + | - | Pre | + | + | + | 2 | T2N2M0 | IIIA | |
| BT84 | NA | F | Caucasian | 1986.062 | Weak | 4888.74 | + | - | NA | + | + | NA | 1 | T1N1M0 | IIA | 9.91 |
| BT50 | 77 | F | Caucasian | 948.234 | Weak | 4887.962 | + | - | Post | + | + | - | 1 | T2N0M0 | IIA | 22 |
| BT93 | 66 | F | Caucasian | 2670.527 | Weak | 4879.083 | + | Weak | Post | + | + | - | 1 | T1N0M0 | I | 0.39 |
| BT67 | 52 | F | African American | 4346.033 | Weak | 4726.79 | + | - | Post | + | + | - | 1 | T2N2M0 | IIIA | 13.11 |
| BT141 | 64 | F | Caucasian | 2526.397 | Weak | 4521.669 | + | Weak | Post | + | + | NA | 1 | T2N1M0 | IIB | 29.34 |
| BT131 | NA | F | Caucasian | 1393.891 | Weak | 4469.376 | + | - | NA | + | + | NA | 1 | T2N1M0 | IIB | 6 |
| BT13 | 47 | F | Caucasian | 612.113 | Weak | 4397.083 | + | - | Pre | + | + | - | 1 | T2N1M0 | IIB | 22.74 |
| BT4 | 37 | F | Caucasian | 2924.79 | Weak | 4393.912 | + | - | NA | + | - | NA | 1 | T2N1MX | IIB | 21.15 |
| BT154 | 37 | F | Caucasian | 1108.841 | Weak | 4264.083 | + | - | Pre | + | + | NA | 1 | T2NXM0 | IIA | 34.64 |
| BT163 | 71 | F | Caucasian | 3511.79 | Weak | 4232.205 | + | - | Post | + | + | NA | 1 | T2NXMX | IIA | 13.29 |
| BT148 | 70 | F | Caucasian | 723.991 | Weak | 4029.426 | + | + | Post | + | + | NA | 1 | T4N2MX | IIIB | 30.75 |
| BT116 | 32 | F | Caucasian | 4004.74 | Weak | 4001.083 | - | - | Pre | + | + | + | 1 | T2N0M0 | IIA | 13.66 |
| BT166 | 69 | F | Caucasian | 1451.598 | Weak | 3962.326 | - | - | Post | + | + | NA | 1 | T1cN1M0 | IIA | 8.38 |
| BT80 | 64 | F | Caucasian | 2250.355 | Weak | 3871.497 | - | - | Post | + | + | - | 1 | T2N1M0 | IIB | 7.33 |
| BT182 | 39 | F | Caucasian | 2108.012 | Weak | 3833.497 | - | - | Pre | + | + | - | 1 | T2NXM0 | IIA | 4.14 |
| BT119 | 42 | F | Caucasian | 1591.012 | Weak | 3781.376 | - | - | Pre | + | + | - | 2 | T2N0M0 | IIA | 24.02 |
| BT96 | 56 | F | Caucasian | 2435.062 | Weak | 3728.083 | - | - | Post | + | + | - | 2 | T2N2M0 | IIIA | 17.57 |
| BT9 | NA | F | Caucasian | 1766.255 | Weak | 3714.79 | - | - | NA | + | + | - | 1 | T1N0M0 | I | 8.79 |
| BT64 | 46 | F | Caucasian | 2420.598 | Weak | 3615.326 | - | - | Pre | + | + | - | 1 | T1cNXM0 | I | 12.57 |
| BT20 | 56 | F | Caucasian | 921.255 | Weak | 3528.669 | - | - | Post | + | + | - | 1 | T1NXMX | I | 21.25 |
| BT40 | 68 | F | Caucasian | 3375.459 | Weak | 3437.962 | - | - | Post | + | + | - | 1 | T4NXM0 | IIIB | 5.7 |
| BT127 | 59 | F | Caucasian | 1539.891 | Weak | 3420.669 | - | Weak | Post | + | - | NA | 1 | T2N1MX | IIB | 5 |
| BT7 | 63 | F | Caucasian | 1852.527 | Weak | 3411.376 | - | + | Post | + | + | - | 2 | T2N0M0 | IIA | |
| BT31 | 55 | F | Caucasian | 2954.288 | Weak | 3394.033 | - | - | Post | + | + | - | 1 | T2NXM0 | IIA | 18 |
| BT107 | 45 | F | Caucasian | 2222.184 | Weak | 3382.083 | - | - | Pre | + | - | - | 1 | T4N1M0 | IIIB | 8.06 |
| BT111 | 65 | F | Caucasian | 2496.134 | Weak | 3376.841 | - | + | Post | + | - | - | 1 | T1N0M0 | I | 39.37 |
| BT55 | 59 | F | Caucasian | 2082.305 | Weak | 3290.669 | - | - | NA | + | + | - | 2 | T1N1M0 | IIA | 6.5 |
| BT82 | 48 | F | Caucasian | 3094.891 | Weak | 3261.619 | - | - | NA | + | + | NA | 1 | T2N2M0 | IIIA | 33.25 |
| BT197 | 64 | F | Caucasian | 2634.012 | Weak | 3216.426 | - | - | Post | + | + | - | 2 | T1cN0M0 | I | |
| BT172 | 44 | F | Caucasian | 576.456 | Weak | 3202.79 | - | - | Pre | + | + | - | 1 | T2N0M0 | IIA | 0.6 |
| BT183 | 52 | F | Asian | 2013.355 | Weak | 3160.376 | - | - | Post | + | + | NA | 1 | T2N2M0 | IIIA | 12.75 |
| BT58 | 65 | F | Caucasian | 3109.276 | Weak | 3106.669 | - | Weak | Post | + | + | - | 2 | T2NXMX | IIA | 8 |
| BT102 | 60 | F | Caucasian | 1414.012 | Weak | 3089.497 | - | - | Post | + | - | - | 1 | T2N1M0 | IIB | 70.97 |
| BT28 | 53 | F | Caucasian | 1753.083 | Weak | 3011.305 | - | - | Post | + | - | - | 1 | T1N1M0 | IIA | 3 |
| BT168 | 53 | F | Caucasian | 896.113 | Weak | 3006.962 | - | - | Post | + | - | NA | 1 | T1cN0MX | I | 9.78 |
| BT101 | 78 | F | Caucasian | 1079.355 | Weak | 2972.083 | - | - | Post | + | + | - | 1 | T2NXM0 | IIA | 11.01 |
| BT47 | 70 | F | Caucasian | 1997.891 | Weak | 2962.305 | - | - | Post | + | + | - | 1 | T1cNXM0 | I | 11.5 |
| BT162 | 47 | F | Caucasian | 2537.548 | Weak | 2892.376 | - | - | NA | + | + | NA | 1 | T1cN0M0 | I | 1.38 |
| BT51 | 47 | F | Caucasian | 510.062 | Weak | 2867.548 | - | Weak | Pre | + | + | - | 1 | T2N0M0 | IIA | 27.88 |
| BT103 | 74 | F | Caucasian | 1194.87 | Weak | 2775.79 | - | - | Post | + | - | - | 1 | T4N1M0 | IIIB | 13.12 |
| BT62 | 45 | F | Caucasian | 2863.962 | Weak | 2762.861 | - | - | Pre | + | + | - | 2 | T2NXMX | IIA | 6.2 |
| BT34 | 51 | F | Caucasian | 1639.234 | Weak | 2708.083 | - | - | Pre | + | + | - | 1 | T2NXM0 | II | 6 |
| BT32 | 39 | F | Caucasian | 805.406 | Weak | 2631.083 | - | - | Pre | + | + | - | 1 | T2NXM0 | IIA | 4 |
| BT114 | NA | F | Caucasian | 1437.456 | Weak | 2599.669 | - | - | Post | + | - | + | 1 | T2N0M0 | IIA | 0.59 |
| BT75 | 51 | F | Caucasian | 1088.406 | Weak | 2549.134 | - | - | Pre | + | + | - | 1 | T2N1M0 | IIB | 27.97 |
| BT100 | 70 | F | Caucasian | 2366.61 | Weak | 2499.548 | - | - + | Post | + | + | - | 1 | T2N0M0 | IIA | 17.41 |
| BT16 | 51 | F | Caucasian | 765.062 | Weak | 2448.548 | - | - | Pre | + | - | - | 1 | T1N0M0 | I | 5 |
| BT193 | 52 | F | Caucasian | 1563.983 | Weak | 2395.255 | - | - | Post | + | - | NA | 1 | T2N0M0 | IIA | 59.92 |
| BT11 | 46 | F | Caucasian | 1592.205 | Weak | 2350.841 | - | - | Pre | + | + | - | 1 | T2N2M0 | IIIA | 13.06 |
| BT30 | 73 | F | Caucasian | 928.941 | Weak | 2306.012 | - | - | Post | + | - | - | 2 | T2NXM0 | IIA | 12.62 |
| BT97 | 51 | F | Caucasian | 2550.083 | Weak | 2270.841 | - | - | Post | + | - | - | 1 | T1NXM0 | I | 18 |
| BT125 | 66 | F | Caucasian | 1740.79 | Weak | 2254.719 | - | Weak | Post | + | - | NA | 1 | T2N1M0 | IIB | 12.98 |
| BT142 | 64 | F | Caucasian | 2102.497 | Weak | 2184.962 | - | Weak | Post | + | + | NA | 3 | T2NXMX | IIA | 8.13 |
| BT147 | 43 | F | Caucasian | 426.456 | Weak | 2130.426 | - | - | Post | + | - | NA | 1 | T4N2MX | IIIB | 3.43 |
| BT60 | 75 | F | Caucasian | 3002.962 | Weak | 2120.426 | - | - | Post | + | - | - | 1 | T2N1M0 | IIB | 7.32 |
| BT69 | 45 | F | Caucasian | 4169.501 | Weak | 2018.134 | - | - | NA | + | - | - | 1 | T2N0M0 | IIA | 12.98 |
| BT25 | 48 | F | Caucasian | 812.234 | Weak | 2011.77 | - | Weak | Pre | + | + | - | 1 | T2NXM0 | IIA | 27.2 |
| BT5 | 49 | F | Caucasian | 667.406 | Weak | 1966.719 | - | - | Pre | + | + | - | 1 | T1cNXM0 | I | |
| BT200 | 66 | F | Caucasian | 1297.246 | Weak | 1881.598 | - | - | Post | + | + | - | 1 | T1N0MX | I | 13.14 |
| BT29 | 52 | F | Caucasian | 612.749 | Weak | 1850.184 | - | - | Pre | + | + | - | 1 | T2NXM0 | IIA | 6.18 |
| BT126 | 51 | F | Caucasian | 1306.719 | Weak | 1825.719 | - | - | Post | + | - | NA | 1 | T2N1MX | IIB | 28.48 |
| BT123 | 69 | F | Caucasian | 689.355 | Weak | 1633.77 | - | - | Pre | + | + | - | 1 | T3N0M0 | IIB | 7.55 |
| BT120 | 55 | F | Caucasian | 1132.234 | Weak | 1622.598 | - | - | Post | + | + | - | 1 | T2N2M0 | IIIA | 6.68 |
| BT6 | 70 | F | Caucasian | 757.669 | Weak | 1460.184 | - | - | Post | + | + | - | 2 | T2N2M0 | IIIA | 10.97 |
| BT98 | 51 | F | Caucasian | 1522.841 | Weak | 1329.255 | - | - | Post | + | + | - | 1 | T1NXM0 | I | 9.07 |
| BT171 | 71 | F | Caucasian | 472.092 | Weak | 838.941 | - | - | Post | + | - | NA | 1 | T1cN2M0 | IIIA | 2.93 |
| BT74 | 37 | F | Caucasian | 453.627 | Weak | 800.82 | - | - | Pre | + | + | - | 1 | T3N1M0 | IIIA | 17.67 |
| BT186 | 56 | F | Caucasian | 374.66 | - | 7750.083 | + | - | Post | + | + | NA | 1 | T3N0M0 | IIB | 20.97 |
| BT46 | 70 | F | Caucasian | 402.589 | - | 6816.376 | + | Weak | Post | + | - | NA | 1 | T2N1M0 | IIB | 17 |
| BT15 | 32 | F | Caucasian | 303.184 | - | 6256.79 | + | - | Pre | + | - | + | 1 | T2NXM0 | IIA | 22.39 |
| BT138 | 68 | F | Caucasian | 364.74 | - | 5969.205 | + | - | Post | + | + | NA | 1 | T2N2M0 | IIIA | 38.32 |
| BT180 | 44 | F | Asian | 330.882 | - | 5606.497 | + | - | Pre | + | - | + | 1 | T2NXMX | IIA | 47.36 |
| BT19 | 53 | F | Caucasian | 302.083 | - | 5401.376 | + | - | Pre | + | + | NA | 1 | T1cN1MX | IIA | 28 |
| BT92 | 69 | F | Caucasian | 358.184 | - | 5362.326 | + | - | Post | + | - | NA | 1 | T1N2M0 | IIIA | 24.07 |
| BT140 | 69 | F | Caucasian | 425.255 | - | 4828.962 | + | - | Post | + | + | NA | 1 | T2NXM0 | IIA | 53.42 |
| BT48 | 56 | F | Caucasian | 363.125 | - | 4435.669 | + | + | NA | + | - | - | 1 | T1NXM0 | I | 28.96 |
| BT153 | 72 | F | Caucasian | 401.861 | - | 4348.497 | + | - | Post | + | + | - | 3 | T1N0M0 | I | 11.84 |
| BT71 | 53 | F | Caucasian | 262.497 | - | 4265.912 | + | - | Pre | + | + | - | 1 | T2N0MX | IIA | 10.62 |
| BT191 | 63 | F | Caucasian | 331.004 | - | 4193.376 | + | Weak | Post | + | - | - | 1 | T3N2M0 | IIIA | 0 |
| BT22 | 40 | F | Caucasian | 279.326 | - | 4088.79 | + | Weak | NA | + | + | - | 1 | T1cN1M0 | IIA | 25.13 |
| BT61 | 79 | F | Caucasian | 235.255 | - | 4058.74 | + | - | Post | + | + | - | 2 | T4N2M0 | IIIB | 19 |
| BT177 | 70 | F | Caucasian | 390.882 | - | 4041.912 | + | - | Post | + | + | NA | 1 | T2N0M0 | IIA | 15.68 |
| BT176 | 42 | F | Caucasian | 367.731 | - | 3878.205 | - | - | Pre | + | + | NA | 1 | T1cNXM0 | I | 56.76 |
| BT178 | 56 | F | Caucasian | 342.882 | - | 3750.033 | - | - | Post | + | + | - | 1 | T1NXM0 | I | 38.34 |
| BT185 | 43 | F | Caucasian | 381.347 | - | 3723.326 | - | - | Pre | + | + | NA | 1 | T2NXM0 | IIA | 11.13 |
| BT161 | 54 | F | Caucasian | 389.782 | - | 3711.376 | - | - | Post | + | + | - | 2 | T2N1M0 | IIB | 20.62 |
| BT158 | 59 | F | Caucasian | 395.539 | - | 3703.083 | - | - | Post | + | + | NA | 1 | T1cNXM0 | I | 13.22 |
| BT167 | 47 | F | Caucasian | 401.731 | - | 3590.083 | - | - | NA | + | + | NA | 1 | T1cN2M0 | IIIA | 0.4 |
| BT188 | 54 | F | Caucasian | 294.439 | - | 3553.426 | - | - | Post | + | + | NA | 2 | T2N0M0 | IIA | 2.12 |
| BT89 | 79 | F | Caucasian | 379.77 | - | 3398.083 | - | - | Post | + | - | NA | 1 | T4N1M0 | IIIB | 31.73 |
| BT129 | 50 | F | Caucasian | 354.841 | - | 3261.619 | - | - | Post | + | - | + | 2 | T2N0M0 | IIA | 19 |
| BT42 | 45 | F | Caucasian | 410.477 | - | 3135.548 | - | - | Pre | + | + | - | 2 | T2N1M0 | IIB | 30 |
| BT156 | 30 | F | Caucasian | 394.569 | - | 3125.669 | - | - | Pre | + | + | NA | 1 | T1N0M0 | I | 8.42 |
| BT17 | 69 | F | Caucasian | 380.468 | - | 2943.669 | - | - | Post | + | + | - | 2 | T2N0M0 | IIA | 16.6 |
| BT179 | 50 | F | Caucasian | 319.125 | - | 2803.205 | - | - | Pre | + | + | NA | 1 | T1cN1M0 | IIA | 22.13 |
| BT187 | 67 | F | Caucasian | 325.731 | - | 2702.548 | - | - | Post | + | - | + | 2 | T1cN0M0 | I | 4.28 |
| BT149 | 37 | F | Caucasian | 312.648 | - | 2692.841 | - | - | Pre | + | + | NA | 1 | T1N0MX | I | 6.7 |
| BT196 | 49 | F | Caucasian | 331.175 | - | 2673.598 | - | + | Post | + | + | - | 2 | T2NOM0 | IIA | 7.98 |
| BT192 | 66 | F | Caucasian | 320.74 | - | 2618.255 | - | - | Post | + | - | - | 2 | T4NXM0 | IIIB | 12.94 |
| BT90 | 88 | F | Caucasian | 329.234 | - | 2606.669 | - | - | Post | + | + | - | 1 | T1N0M0 | I | 10.99 |
| BT169 | NA | F | Caucasian | 407.246 | - | 2555.255 | - | - | Post | + | + | NA | 3 | T1N0M0 | I | 12.42 |
| BT41 | 41 | F | Caucasian | 232.234 | - | 2487.841 | - | - | Pre | + | + | - | 1 | T2NXM0 | IIA | 20.7 |
| BT18 | 53 | F | Caucasian | 407.569 | - | 2410.134 | - | - | Pre | + | + | - | 1 | T2N0M0 | IIA | 2.06 |
| BT12 | NA | F | Caucasian | 274.205 | - | 2355.012 | - | Weak | Post | + | - | - | 1 | T1cN1MX | IIA | 7 |
| BT83 | 47 | F | Caucasian | 469.134 | - | 2337.669 | - | - | NA | + | - | + | 1 | T1cN0M0 | I | 6.43 |
| BT117 | 59 | F | Caucasian | 406.669 | - | 2275.548 | - | - | Post | + | + | - | 2 | T2N0M0 | IIA | 12 |
| BT157 | 48 | F | Caucasian | 341.225 | - | 2263.841 | - | - | Post | + | + | NA | 1 | T1cN0M0 | I | 10.3 |
| BT10 | 74 | F | Caucasian | 302.962 | - | 2235.012 | - | - | Post | + | + | - | 1 | T2NXM0 | IIA | 6 |
| BT105 | 58 | F | Caucasian | 352.083 | - | 2225.548 | - | - | Pre | + | + | - | 1 | T1cN1M0 | IIA | 2 |
| BT27 | 74 | F | Caucasian | 365.113 | - | 2207.477 | - | - | Post | + | - | - | 2 | T1N1M0 | IIA | 3.81 |
| BT124 | 50 | F | Caucasian | 335.569 | - | 2198.669 | - | - | Post | + | + | - | 1 | T2NOM0 | IIA | 17.25 |
| BT99 | 39 | F | Caucasian | 417.74 | - | 2173.012 | - | - | Pre | + | + | NA | 1 | T2NXM0 | IIA | 23.87 |
| BT68 | 68 | F | Caucasian | 350.477 | - | 2168.548 | - | - | Post | + | - | - | 1 | T3N1M0 | IIIA | 0.99 |
| BT91 | 45 | F | Caucasian | 313.305 | - | 2100.669 | - | - | Pre | + | + | - | 1 | T2N2M0 | IIIA | 10.66 |
| BT134 | 75 | F | Caucasian | 376.355 | - | 2068.184 | - | - | Post | + | + | NA | 1 | T2NXMX | IIA | 0.6 |
| BT190 | 51 | F | Caucasian | 386.095 | - | 1981.719 | - | - | Post | + | + | + | 1 | T2N2M0 | IIIA | 17.59 |
| BT77 | 56 | F | Caucasian | 423.134 | - | 1922.426 | - | - | NA | + | + | - | 1 | T2N1M0 | IIB | 6.76 |
| BT73 | 50 | F | Caucasian | 408.962 | - | 1895.062 | - | - | Pre | + | + | - | 1 | T2N3M0 | IIIC | 10.17 |
| BT54 | 42 | F | Caucasian | 362.962 | - | 1822.012 | - | - | Pre | + | - | - | 1 | T1N0M0 | I | 2.12 |
| BT133 | 51 | F | Caucasian | 398.497 | - | 1801.426 | - | - | Post | + | + | + | 1 | T1N0M0 | I | 28.46 |
| BT112 | 41 | F | Caucasian | 373.205 | - | 1766.648 | - | - | Pre | + | + | - | 2 | T2N0M0 | IIA | 0.99 |
| BT66 | 43 | F | Caucasian | 373.426 | - | 1681.012 | - | - | Pre | + | + | - | 1 | T2N0M0 | IIA | 0.6 |
| BT8 | 51 | F | Caucasian | 334.184 | - | 1618.648 | - | - | NA | + | - | - | 2 | T1N0M0 | I | 0.4 |
| BT2 | 68 | F | Caucasian | 409.598 | - | 1613.891 | - | - | Post | + | - | - | 2 | T1cNXM0 | I | 12.98 |
| BT81 | 67 | F | Caucasian | 390.497 | - | 1590.426 | - | - | Post | + | + | - | 2 | T2N0M0 | IIA | 0.2 |
| BT115 | 61 | F | Caucasian | 381.305 | - | 1568.598 | - | - | Post | + | + | - | 1 | T2NOMX | IIA | 0.6 |
| BT128 | 50 | F | Caucasian | 352.447 | - | 1510.477 | - | - | Post | + | + | NA | 2 | T1N1M0 | IIA | 12 |
| BT136 | 77 | F | Caucasian | 413.255 | - | 1505.941 | - | - | Post | + | + | NA | 1 | T1NXM0 | I | 6.74 |
| BT94 | 54 | F | Caucasian | 350.962 | - | 1473.891 | - | - | Pre | + | + | - | 3 | T4N3M0 | IIIC | 7.14 |
| BT85 | NA | F | Caucasian | 403.648 | - | 1461.012 | - | - | NA | + | - | NA | 1 | T2NXM0 | IIA | 11.05 |
| BT199 | 53 | F | Caucasian | 303.184 | - | 1460.134 | - | - | Post | + | + | + | 1 | T4N1M0 | IIIB | 0.96 |
| BT152 | 86 | F | Caucasian | 309.426 | - | 1440.184 | - | - | Post | + | - | - | 1 | T2NXM0 | IIA | 0.4 |
| BT118 | 43 | F | Caucasian | 328.598 | - | 1398.941 | - | - | Post | + | + | - | 1 | T4N1M0 | IIIB | 9.26 |
| BT76 | 55 | F | Caucasian | 294.899 | - | 1327.355 | - | - | Post | + | + | - | 1 | T2N0M0 | IIA | 10.1 |
| BT43 | 74 | F | Caucasian | 347.376 | - | 1248.648 | - | - | Post | + | + | NA | 1 | T2N1M0 | IIB | 22 |
| BT150 | 67 | F | Caucasian | 322.79 | - | 1165.648 | - | - | Post | + | + | NA | 1 | T2N1M0 | IIB | 4.44 |
| BT53 | 36 | F | Asian | 326.426 | - | 1107.355 | - | - | Pre | NA | NA | NA | 1 | | | |
| BT175 | 37 | F | Caucasian | 406.213 | - | 988.355 | - | - | Pre | + | + | NA | 1 | T1cN1M0 | IIA | 27.07 |
| BT170 | 70 | F | Caucasian | 436.66 | - | 978.527 | - | - | Post | + | + | - | 2 | T2N3M0 | IIIC | 2.96 |
| BT39 | 44 | F | Caucasian | 422.376 | - | 969.234 | - | - | Pre | + | + | - | 1 | T1N1M0 | IIA | 32.5 |
| BT65 | 59 | F | Caucasian | 325.205 | - | 813.406 | - | - | Post | + | + | - | 1 | T1NXM0 | I | 11.4 |
| BT38 | 62 | F | Caucasian | 472.205 | - | 715.113 | - | - | Post | + | - | NA | 1 | T1cNXM0 | I | 13.44 |
| BT146 | 65 | F | Caucasian | 377.991 | - | 708.406 | - | - | Post | + | + | NA | 2 | T2N1M0 | IIB | 6.24 |
| BT155 | 68 | F | Caucasian | 376.64 | - | 688.627 | - | - | Post | + | - | + | 2 | T1cN0M0 | I | 2.95 |
| BT122 | 53 | F | Caucasian | 312.154 | - | 562.456 | - | - | Post | + | + | - | 2 | T2NOM0 | IIA | |
| BT87 | 53 | F | Caucasian | 374.941 | - | 558.092 | - | - | Post | + | + | - | 1 | T1NXM0 | I | 18.34 |
| BT1 | 75 | F | Caucasian | 439.012 | - | 514.749 | - | - | Post | + | + | - | 1 | T2NXM0 | IIA | 9.73 |
| BT174 | 45 | F | Caucasian | 402.397 | - | 441.627 | - | - | NA | + | - | - | 3 | T1cN0M0 | I | 0.99 |
| BT145 | 53 | F | Caucasian | 367.012 | - | 406.627 | - | - | Post | + | + | NA | 1 | T1cNXMX | IC | 27.85 |
| BT173 | 63 | F | Caucasian | 345.368 | - | 400.263 | - | - | Post | + | + | + | 1 | T1cN0M0 | I | 13.27 |
| BT121 | 72 | F | Caucasian | 321.912 | - | 394.506 | - | - | Post | + | + | - | 1 | T2NXM0 | IIA | 10.97 |
| BT49 | 47 | F | Caucasian | 244.305 | - | 373.627 | - | - | Post | + | + | - | 1 | T2N2M | IIIA | 32 |
| BT63 | 79 | F | Caucasian | 259.255 | - | 330.607 | - | - | Post | + | + | - | 2 | T2NXM0 | IIA | 18.73 |
| BT3 | 64 | F | Caucasian | 325.962 | - | 289.142 | - | - | Post | + | - | - | 2 | T1NXM0 | I | 0.4 |
| BT86 | 81 | F | Caucasian | 326.113 | - | 254.607 | - | - | Post | + | + | - | 1 | T2N1aM0 | IIB | 0.2 |

The Ki67 scores are derived from our previous study (Nat Commun. 2014; 5:4577). MP stat, Menopausal status. NA, Not Available.

TABLE 4

Primer sequences and amplification conditions used in RT-PCR or real-time PCR analyses
RT-PCR

| Gene | RAD51AP1-DYRK4 | |
|---|---|---|
| Primers sequence | Forward | 5′-CAAGCCTTGAAAGGGACCAT-3′ (SEQ ID NO:5) |
| | Reverse | 5′-CCAACCTTCGCAGAGGTGAA-3′ (SEQ ID NO:6) |
| PCR amplification conditions | 1 cycle | 94° C.: 2 minutes |
| | 35 cycles | 94° C.: 15 seconds |
| | | 60° C.: 30 seconds |
| | | 68° C.: 2 minutes |
| | 1 cycle | 68° C.: 5 minutes |
| Gene | wtRADS1AP1 | |
| Primers sequence | Forward | 5′-GCCGTCAAATCAGAATCTCAGTC-3′ (SEQ ID NO:13) |
| | Reverse | 5′-AAGCTGTGATTCTCCCAACCAA-3′ (SEQ ID NO:14) |
| PCR amplification conditions | 1 cycle | 94° C.: 2 minutes |
| | 35 cycles | 94° C.: 15 seconds |
| | | 60° C.: 30 seconds |
| | | 68° C.: 2 minutes |
| | 1 cycle | 68° C.: 5 minutes |
| Gene | wtDYRK4 | |
| Primers sequence | Forward | 5′-CCAGACCCTGAGGAAATCCA-3′ (SEQ ID NO:11) |
| | Reverse | 5′-CTGACTTCTTGGGAGCGTCT-3′ (SEQ ID NO:12) |
| PCR amplification conditions | 1 cycle | 94° C.: 2 minutes |
| | 35 cycles | 94° C.: 15 seconds |
| | | 60° C.: 30 seconds |
| | | 72° C.: 2 minutes |
| | 1 cycle | 72° C.: 5 minutes |
| Primers sequence | Forward | 5′-CCCACTCCTCCACCTTTGAC-3′ (SEQ ID NO:17) |
| | Reverse | 5′-TCCTCTTGTGCTCTTGCTGG-3′ (SEQ ID NO:18) |
| | 1 cycle | 94° C.: 2 minutes |
| | | 94° C.: 15 seconds |
| PCR amplification conditions | 30 cycles | 60° C.: 30 seconds |
| | | 72° C.: 2 minutes |
| | 1 cycle | 72° C.: 5 minutes |
| | Forward | 5′-AGCTGCCGTCAAATCAGAAT-3′ (SEQ ID NO:7) |
| Primers sequence | Reverse | 5′-CCAGGAAGGGAGTCAAATCA-3′ (SEQ ID NO:8) |
| | 1 cycle | 94° C.: 10 minutes |
| | | 94° C.: 15 seconds |
| PCR amplification conditions | 40 cycles | 60° C.: 30 seconds |
| | | 68° C.: 1 minutes |
| | Melting curve | 95° C.: 15 seconds, 60° C.: 1 minute, 95° C.: 15 seconds. |
| Primers sequence | Forward | 5′-TGTGAGAGTGAGGATAATGACGAA-3′ (SEQ ID NO:25) |
| | Reverse | 5′-CCAGGAAGGGAGTCAAATCACA-3′ (SEQ ID NO:26) |
| | 1 cycle | 94° C.: 10 minutes |
| | | 94° C.: 15 seconds |
| PCR amplification conditions | 40 cycles | 60° C.: 30 seconds |
| | | 68° C.: 1 minutes |
| | Melting curve | 95° C.: 15 seconds, 60° C.: 1 minute, 95° C.: 15 seconds. |
| | Forward | 5′-GCCGTCAAATCAGAATCTCAGTC-3′ (SEQ ID NO:13) |
| Primers sequence | Reverse | 5′-AAGCTGTGATTCTCCCAACCAA-3′ (SEQ ID NO:14) |
| | 1 cycle | 94° C.: 10 minutes |
| | | 94° C.: 15 seconds |
| PCR amplification conditions | 40 cycles | 60° C.: 30 seconds |
| | | 68° C.: 1 minutes |
| | Melting curve | 95° C.: 15 seconds, 60° C.: 1 minute, 95° C.: 15 seconds. |
| | Forward | 5′-GCACCGGAACAAAGACTCAA-3′ (SEQ ID NO:15) |
| Primers sequence | Reverse | 5′-ACTTGGGTCATTCTGGAGTTCT-3′ (SEQ ID NO:16) |
| PCR amplification conditions | 1 cycle | 94° C.: 10 minutes |
| | 40 cycles | 94° C.: 15 seconds |
| | | 60° C.: 30 seconds |
| | | 68° C.: 1 minutes |
| | Melting curve | 95° C.: 15 seconds, 60° C.: 1 minute, 95° C.: 15 seconds. |
| Primers sequence | Forward | 5′-CCCACTCCTCCACCTTTGAC-3′ (SEQ ID NO:17) |
| | Reverse | 5′-TCCTCTTGTGCTCTTGCTGG-3′ (SEQ ID NO:18) |
| PCR amplification conditions | 1 cycle | 94° C.: 10 minutes |
| | 40 cycles | 94° C.: 15 seconds |
| | | 60° C.: 30 seconds |
| | | 68° C.: 1 minutes |
| | Melting curve | 95° C.: 15 seconds, 60° C.: 1 minute, 95° C.: 15 seconds. |

TABLE 5

Primary antibodies used in western blots

| Name | Manufacturer | Catalog no. | Species | Type | Clone |
|---|---|---|---|---|---|
| RAD51AP1 | GeneTex | GTX115455 | Rabbit | Polyclonal | N1C1 |
| MAP3K1 | Bethyl | A302-395A | Rabbit | Polyclonal | |
| ITGB1 | Cell Signaling Technology | 9699 | Rabbit | Polyclonal | D2E5 |
| Anti-Flag | Cell Signaling Technology | 8146 | Mouse | Monoclonal | 9A3 |
| GAPDH | Santa Cruz | sc-32233 | Mouse | Monoclonal | 6C5 |
| ORC2 | BD Biosciences | 559266 | Rabbit | Polyclonal | |
| HER2 | Cell Signaling Technology | 4290 | Rabbit | Polyclonal | D8F12 |
| pHER2(Y1248) | Cell Signaling Technology | 2247 | Rabbit | Polyclonal | |
| PI3K-P85 | Cell Signaling Technology | 4257 | Rabbit | Polyclonal | 19H8 |
| pPI3K-P85 | Cell Signaling Technology | 4228 | Rabbit | Polyclonal | |
| AKT | Cell Signaling Technology | 4691 | Rabbit | Polyclonal | C67E7 |
| pAKT(S473) | Cell Signaling Technology | 4060 | Rabbit | Polyclonal | D9E |
| pAKT(T308) | Cell Signaling Technology | 4056 | Rabbit | Polyclonal | 244F9 |
| Raptor | Cell Signaling Technology | 2280 | Rabbit | Polyclonal | 24C12 |
| pRaptor(S863) | Signalway | 12778-1 | Rabbit | Polyclonal | |
| ERK | Cell Signaling Technology | 4695 | Rabbit | Polyclonal | 137F5 |
| pERK(T202/Y20 4) | Cell Signaling Technology | 4370 | Rabbit | Polyclonal | D13.14.4E |
| MEK½ | Cell Signaling Technology | 9122 | Rabbit | Polyclonal | |
| pMEK½(S217/2 21) | Cell Signaling Technology | 9154 | Rabbit | Polyclonal | 41G9 |

### REFERENCES

1. Yersal, O. & Barutca, S. Biological subtypes of breast cancer: Prognostic and therapeutic implications. World J Clin Oncol 5, 412-424 (2014).

2. Goksu, S.S. et al. Clinicopathologic features and molecular subtypes of breast cancer in young women (age</=35). Asian Pac J Cancer Prev 15, 6665-6668 (2014).

3. Ades, F. et al. Luminal B breast cancer: molecular characterization, clinical management, and future perspectives. J Clin Oncol 32, 2794-2803 (2014).

4. Sotiriou, C. & Pusztai, L. Gene-expression signatures in breast cancer. The New England journal of medicine 360, 790-800 (2009).

5. Koboldt, D.C. et al. Comprehensive molecular portraits of human breast tumours. Nature (2012).

6. Veeraraghavan, J. et al. Recurrent ESR1-CCDC170 rearrangements in an aggressive subset of oestrogen receptor- positive breast cancers. Nat Commun 5, 4577 (2014).

7. Fimereli, D. et al. Genomic hotspots but few recurrent fusion genes in breast cancer. Genes Chromosomes Cancer 57, 331-338 (2018).

8. Giltnane, J.M. et al. Genomic profiling of ER(+) breast cancers after short-term estrogen suppression reveals alterations associated with endocrine resistance. Sci Transl Med 9 (2017).

9. Matissek, K.J. et al. Expressed Gene Fusions as Frequent Drivers of Poor Outcomes in Hormone Receptor- Positive Breast Cancer. Cancer Discov 8, 336-353 (2018).

10. Hartmaier, R.J. et al. Recurrent hyperactive ESR1 fusion proteins in endocrine therapy-resistant breast cancer. Ann Oncol 29, 872-880 (2018).

11. Kim, J.A. et al. Comprehensive functional analysis of the tousled-like kinase 2 frequently amplified in aggressive luminal breast cancers. Nat Commun 7, 12991 (2016).

12. Wang, X.S. et al. An integrative approach to reveal driver gene fusions from paired-end sequencing data in cancer. Nat Biotechnol 27, 1005-1011 (2009).

13. Wiese, C. et al. Promotion of homologous recombination and genomic stability by RAD51AP1 via RAD51 recombinase enhancement. Molecular cell 28, 482-490 (2007).

14. Dunlop, M.H. et al. RAD51-associated protein 1 (RAD51AP1) interacts with the meiotic recombinase DMC1 through a conserved motif. The Journal of biological chemistry 286, 37328-37334 (2011).

15. Obama, K. et al. Enhanced expression of RAD51 associating protein-1 is involved in the growth of intrahepatic cholangiocarcinoma cells. Clin Cancer Res 14, 1333-1339 (2008).

16. Park, J., Song, W.J. & Chung, K.C. Function and regulation of Dyrk1A: towards understanding Down syndrome. Cellular and molecular life sciences : CMLS 66, 3235-3240 (2009).

17. Wang, X. et al. Epigenetic activation of HORMAD1 in basal-like breast cancer: role in Rucaparib sensitivity. Oncotarget 9, 30115-30127 (2018).

18. Watkins, J. et al. Genomic Complexity Profiling Reveals That HORMAD1 Overexpression Contributes to Homologous Recombination Deficiency in Triple-Negative Breast Cancers. Cancer Discov 5, 488-505 (2015).

19. Mahmoud, A.M. Cancer testis antigens as immunogenic and oncogenic targets in breast cancer. Immunotherapy 10, 769-778 (2018).

20. Sequist, L.V. et al. Implementing multiplexed genotyping of non-small-cell lung cancers into routine clinical practice. Annals of oncology: official journal of the European Society for Medical Oncology / ESMO 22, 2616- 2624 (2011).

21. Cheang, M.C. et al. Ki67 index, HER2 status, and prognosis of patients with luminal B breast cancer. Journal of the National Cancer Institute 101, 736-750 (2009).

22. Voduc, K.D. et al. Breast cancer subtypes and the risk of local and regional relapse. Journal of clinical oncology: official journal of the American Society of Clinical Oncology 28, 1684-1691 (2010).

23. Tran, B. & Bedard, P.L. Luminal-B breast cancer and novel therapeutic targets. Breast cancer research: BCR 13, 221 (2011).

24. Voura, E.B., Ramjeesingh, R.A., Montgomery, A.M. & Siu, C.H. Involvement of integrin alpha(v)beta(3) and cell adhesion molecule L1 in transendothelial migration of melanoma cells. Molecular biology of the cell 12, 2699- 2710 (2001).

25. Streuli, C.H. et al. Laminin mediates tissue-specific gene expression in mammary epithelia. The Journal of cell biology 129, 591-603 (1995).

26. Pham, T.T., Angus, S.P. & Johnson, G.L. MAP3K1: Genomic Alterations in Cancer and Function in Promoting Cell Survival or Apoptosis. Genes Cancer 4, 419-426 (2013).

27. Engel, L.W. & Young, N.A. Human breast carcinoma cells in continuous culture: a review. Cancer Res 38, 4327- 4339 (1978).

28. Antoon, J.W., White, M.D., Driver, J.L., Burow, M.E. & Beckman, B.S. Sphingosine kinase isoforms as a therapeutic target in endocrine therapy resistant luminal and basal-A breast cancer. Exp Biol Med (Maywood) 237, 832-844 (2012).

29. Goel, S. et al. Overcoming Therapeutic Resistance in HER2-Positive Breast Cancers with CDK4/6 Inhibitors. Cancer cell 29, 255-269 (2016).

30. Xue, Z. et al. MAP3K1 and MAP2K4 mutations are associated with sensitivity to MEK inhibitors in multiple cancer models. Cell Res 28, 719-729 (2018).

31. Wee, S. et al. PI3K pathway activation mediates resistance to MEK inhibitors in KRAS mutant cancers. Cancer Res 69, 4286-4293 (2009).

32. Avivar-Valderas, A. et al. Functional significance of co-occurring mutations in PIK3CA and MAP3K1 in breast cancer. Oncotarget 9, 21444-21458 (2018).

33. Zhang, Y. et al. Chimeric transcript generated by cis-splicing of adjacent genes regulates prostate cancer cell proliferation. Cancer discovery 2, 598-607 (2012).

34. Maher, C.A. et al. Transcriptome sequencing to detect gene fusions in cancer. Nature 458, 97-101 (2009).

35. Nacu, S. et al. Deep RNA sequencing analysis of readthrough gene fusions in human prostate adenocarcinoma and reference samples. BMC medical genomics 4, 11 (2011).

36. Li, J. et al. TCPA: a resource for cancer functional proteomics data. Nature methods 10, 1046-1047 (2013).

37. Cen, J. et al. Exosomal Thrombospondin-1 Disrupts the Integrity of Endothelial Intercellular Junctions to Facilitate Breast Cancer Cell Metastasis. Cancers (Basel) 11 (2019).

What is claimed is:

1. A method of diagnosing a subject with increased sensitivity to a MEK inhibitor comprising:
a. obtaining a biological sample from the subject; and
b. detecting an RAD51AP1-DYRK4 gene fusion in the sample, wherein the detection indicates the subject has increased sensitivity to the MEK inhibitor and the subject is diagnosed with increased sensitivity to the MEK inhibitor.

2. The method of claim 1, wherein the RAD51AP1-DYRK4 gene fusion is selected from the group consisting of an E9-E2 fusion, an E8-E2 fusion, and an E8s-E2 fusion.

3. The method of claim 2, wherein the E9-E2 fusion is an mRNA transcript comprising a sequence corresponding to SEQ ID NOs: 28-33, SEQ ID NO:35 and SEQ ID NOs: 38-51, the E8-E2 fusion is an mRNA transcript comprising a sequence corresponding to SEQ ID NOs: 28-33, and SEQ ID NOs: 38-51, and the E8s-E2 fusion is an mRNA transcript comprising a sequence corresponding to SEQ ID NOs: 28-32, SEQ ID NO: 34, and SEQ ID NOs: 38-51.

4. The method of claim 3, wherein the detection comprises contacting the biological sample with a reaction mixture comprising a probe specific for a fusion point nucleotide sequence in at least one of SEQ ID NO: 52, SEQ ID NO: 53 and SEQ ID NO:54.

5. The method of claim 1, wherein the detection comprises contacting the biological sample with a reaction mixture comprising two primers, wherein the first primer is complementary to a RAD51AP1 polynucleotide sequence and the second primer is complementary to a DYRK4 polynucleotide sequence, wherein the RAD51AP1-DYRK4 gene fusion is detectable by the presence of an amplicon generated by the first primer and the second primer.

6. The method of claim 1, wherein the detection comprises contacting the biological sample with a reaction mixture comprising two probes, wherein the first probe is complementary to a RAD51AP1 polynucleotide sequence and the second probe is complementary to a DYRK4 polynucleotide sequence, wherein hybridization of the two probes on a RAD51AP1-DYRK4 gene fusion sequence provides a detectable signal, and the RAD51AP1-DYRK4 gene fusion is detectable by the presence of the signal.

7. The method of claim 5, wherein a first of the one or more primers or probes is selected from the group consisting of SEQ ID NO: 5, SEQ ID NO:7 and SEQ ID NO;25 and a second of the one or more primers or probes is selected from the group consisting of SEQ ID NO: 6, SEQ ID NO: 8 and SEQ ID NO:26.

8. The method of claim 5, wherein the primers are SEQ ID NO: 5 and SEQ ID NO: 6.

9. The method of claim 5, wherein the primers are SEQ ID NO: 7 and SEQ ID NO: 8.

10. The method of claim 5, wherein the primers are SEQ ID NO: 26 and SEQ ID NO: 27.

11. The method of claim 1, wherein the subject has a cancer.

12. The method of claim 11, wherein the subject has a breast cancer.

13. The method of claim 12, wherein the subject has a luminal B or metastatic breast cancer.

14. The method claim 1, wherein the detection of the RAD51AP1-DYRK4 gene fusion indicates an increased sensitivity to one or more of trametinib, cobimetinib, binimetinib, selumetinib, Refametinib, Pimasertib, RO4987655, RO5126766, WX-554, HL-085, PD-325901, PD184352, AZD8330, TAK-733 and GDC-0623.

15. The method claim 1, further comprising administering to the subject a therapeutically effective amount of a MEK inhibitor.

16. The method of claim 15, wherein the MEK inhibitor is trametinib.

17. A method of treating a cancer in a subject comprising:
a. detecting an RAD51AP1-DYRK4 gene fusion in a sample obtained from the subject;
b. administering to the subject a therapeutically effective amount of a MEK inhibitor.

18. The method of claim 17, wherein the RAD51AP1-DYRK4 gene fusion is selected from the group consisting of an E9-E2 fusion, an E8-E2 fusion, and an E8s-E2 fusion.

19. The method of claim 18, wherein the E9-E2 fusion is an mRNA transcript comprising a sequence corresponding to SEQ ID NOs: 28-33, SEQ ID NO:35 and SEQ ID NOs: 38-51, the E8-E2 fusion is an mRNA transcript comprising a sequence corresponding to SEQ ID NOs: 28-33, and SEQ ID NOs: 38-51, and the E8s-E2 fusion is an mRNA transcript comprising a sequence corresponding to SEQ ID NOs: 28-32, SEQ ID NO: 34, and SEQ ID NOs: 38-51.

20. The method of claim 17, wherein the subject has a breast cancer.

21. The method of claim 20, wherein the subject has a luminal B or metastatic breast cancer.

22. The method of claim 17, wherein the sample is a breast tissue sample.

23. The method of claim 17, wherein the MEK inhibitor is trametinib, cobimetinib, binimetinib, selumetinib, Refametinib, Pimasertib, RO4987655, RO5126766, WX-554, HL-085, PD-325901, PD184352, AZD8330, TAK-733 or GDC-0623.

24. The method of claim 17, wherein the MEK inhibitor is trametinib.

25. A method of detecting an RAD51AP1-DYRK4 gene fusion comprising:
a. obtaining a biological sample from a subject; and
b. detecting the fusion in the sample.

26. The method of claim 25, wherein the detection comprises contacting the biological sample with a reaction mixture comprising a probe specific for a fusion point nucleotide sequence in at least one of SEQ ID NO: 52, SEQ ID NO:53 and SEQ ID NO:54.

27. The method of claim 26, wherein a detectable moiety is covalently bonded to the probe.

28. A kit comprising one or more probes, wherein each probe specifically hybridizes to a fusion point nucleotide sequence within SEQ ID NO: 52, SEQ ID NO: 53, or SEQ ID NO:54.

29. The kit of claim 28, wherein a detectable moiety is covalently bonded to the probe.


SEQUENCE LISTING (57 sequences)

SEQ ID NO: 1 - protein, 352 aa, Homo sapiens
MVRPVRHKKPVNYSQFDHSDSDDDFVSATVPLNKKSRTAPKELKQDKPKPNLNNLRKEEIPVQEKTPKKRLPEGTFSIPASAVPCTKMALDDKLYQRDLEVALALSVKELPTVTTNVQNSQDKSIEKHGSSKIETMNKSPHISNCSVASDYLDLDKITVEDDVGGVQGKRKAASKAAAQQRKILLEGSDGDSANDTEPDFAPGEDSEDDSDFCESEDNDEDFSMRKSKVKEIKKKEVKVKSPVEKKEKKSKSKCNALVTSVDSAPAAVKSESQSLPKKVSLSSDTTRKPLEIRSPSAESKKPKWVPPAASGGSRSSSSPLVVVSVKSPNQSLRLGLSRLARVKPLHPNATST

SEQ ID NO: 2 - DNA, 2163 bp, Homo sapiens
gaagccaacaagaatttgagaactgtaaataccaagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaagactccctgaaggtacttttagtattccagctagtgcagtgccttgtacaaagatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttggtgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccagcggcatctggaggtagcagaagtagcagcagcccactggtggtagtgtctgtgaagtctcccaatcagagtctccgccttggcttgtccagattagcacgagttaaacctttgcatccaaatgccactagcacctgagtgtggtacaggaggaatgtttggttgggagaatcacagctttacaagggtgtttatatttgatttgtgtttatatttgaggcaggtattgtaatataaaggaatccattaccatgtcctataaatgacctctagccattttatgattatgttctctgtaaaactcttcaagacttcaatgagaagtttgtttataagaattatcttctcatacctttccttgtgaagagcgtattctgtttttctatcagttcgacatgaagtccacatcacatgctgttcttttctagttacatgatgtgcctttctagctttgtctagtttatagcaccttaactttaactgttcagttttatctggcagaggaaaacattcttatttctttcagaagacatttctgaaatcttataagctacttaagctacgttgtcagttttatcgcaaagatgttttgtattttagccaaatctttttatagtacaaacttagaattattttacacactaaaatggttgcagttttatggcatatgtctccgatttagatggttattctctagaaaatagtatttaaagacattttatgaaatcttcattgtcaaaacctttaataaaagtggaaatattttgaaatgccctttttcttgataccactcatccacgtgttcctgattgtccacatttcatgataaaatgagagctccgcagagaatgttagcctttctgttgtaaatgtaatcttcaagtagtcactttttgttaagttctttagaaagtagttgtcaagtacttagtcatccctattatgatatgagatagtacagcttttcaggaagcttagatctgaatttactttgaaaaacaattgtaatgaatattttatatttacattgagaatttcaactagcttctgatcaatttttaataaaaaattttcaaatcatgttagctgttaaaaaatgtataataactcagtttttcttggtttatggaaatatctatattaatgtgaaaataattaatttagaattgtgattaaagtgagcatttgtcta

SEQ ID NO: 3 - protein, 520 aa, Homo sapiens
MPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRCLVWEPSLRMTPDQALKHAWIHQSRNLKPQPRPQTLRKSNSFFPSETRKDKVQGCHHSSRKADEITKETTEKTKDSPTKHVQHSGDQQDCLQHGADTVQLPQLVDAPKKSEAAVGAEVSMTSPGQSKNFSLKNTNVLPPIV

SEQ ID NO: 4 - DNA, 1864 bp, Homo sapiens
agagtcagtgtgagctgttgaaagcctgcagctaaacaccagtgttacttcactcccctttgtggacaccaaggggaagaagaatacggtaagcttcccacacattagcaagaaagtcctgctgaagtcatccctgctgtatcaggagaatcaagctcacaatcagatgccggcctcagagctcaaggcttcagaaatacctttccaccctagcattaaaacccaggatcccaaggcagaggagaagtcaccaaagaagcaaaaggtgactctgacagcggcagaggccctaaagctttttaagaaccagctgtctccatatgaacaaagtgaaatcctgggctacgcggagctgtggttcctgggtcttgaagccaagaagctcgacacggctcctgagaaatttagcaagacgagttttgatgatgagcatggcttctatctgaaggtcctgcatgatcacattgcctaccgctatgaagttctggagacaatcgggaaggggtcctttggacaggtggccaagtgcttggatcacaaaaacaatgagctggtggccctgaaaatcatcaggaacaagaagaggtttcaccagcaggccctgatggagctgaagatcctggaagctctcagaaagaaggacaaagacaacacctacaatgtggtgcatatgaaggactttttctactttcgcaatcacttctgcatcacctttgagctcctgggaatcaacttgtatgagttgatgaagaataacaactttcaaggcttcagtctgtccatagttcggcgcttcactctctctgttttgaagtgcttgcagatgctttcggtagagaaaatcattcactgtgatctcaagcccgaaaatatagtgctataccaaaagggccaagcctctgttaaagtcattgactttggatcaagctgttatgaacaccagaaagtatacacgtacatccaaagccggttctaccgatccccagaagtgatcctgggccacccctacgacgtggccattgacatgtggagcctgggctgcatcacggcggagttgtacacgggctaccccctgttccccggggagaatgaggtggagcagctggcctgcatcatggaggtgctgggtctgccgccagccggcttcattcagacagcctccaggagacagacattctttgattccaaaggttttcctaaaaatataaccaacaacagggggaaaaaaagatacccagattccaaggacctcacgatggtgctgaaaacctatgacaccagcttcctggactttctcagaaggtgtttggtatgggaaccttctcttcgcatgaccccggaccaggccctcaagcatgcttggattcatcagtctcggaacctcaagccacagcccaggccccagaccctgaggaaatccaattcctttttcccctctgagacaaggaaggacaaggttcaaggctgtcatcactcgagcagaaaagcagatgagatcaccaaagagactacagagaaaacaaaagatagccccacgaagcatgttcagcattcaggtgatcagcaggactgtctccagcacggagctgacactgttcagctgcctcaactggtagacgctcccaagaagtcagaggcagctgtcggggcggaggtgtccatgacctccccaggacagagcaaaaacttctccctcaagaacacaaacgttttaccccctattgtatgacctttgctgagggtatgtcctgctcctttccaccagtgatttgtattaagacagcacttatattgtacaatacttcagactgttttttttaaatacataaaactttatgttaaaaaactctattaacatggccaa

SEQ ID NO: 5 - DNA, 20 bp, Artificial Sequence
  Synthetic construct
caagccttgaaagggaccat

SEQ ID NO: 6 - DNA, 20 bp, Artificial Sequence
  Synthetic construct
ccaaccttcgcagaggtgaa

SEQ ID NO: 7 - DNA, 20 bp, Artificial Sequence
  Synthetic construct
agctgccgtcaaatcagaat

SEQ ID NO: 8 - DNA, 20 bp, Artificial Sequence
  Synthetic construct
ccaggaagggagtcaaatca

SEQ ID NO: 9 - protein, 393 aa, Homo sapiens [sequence omitted]

SEQ ID NO: 10 - protein, 400 aa, Homo sapiens [sequence omitted]

SEQ ID NO: 11 - DNA, 20 bp, Artificial Sequence
  Synthetic construct
ccagaccctgaggaaatcca

SEQ ID NO: 12 - DNA, 20 bp, Artificial Sequence
  Synthetic construct
ctgacttcttgggagcgtct

SEQ ID NO: 13 - DNA, 23 bp, Artificial Sequence
  Synthetic construct
gccgtcaaatcagaatctcagtc

SEQ ID NO: 14 - DNA, 22 bp, Artificial Sequence
  Synthetic construct
aagctgtgattctcccaaccaa

SEQ ID NO: 15 - DNA, 20 bp, Artificial Sequence
  Synthetic construct
gcaccggaacaaagactcaa

SEQ ID NO: 16 - DNA, 22 bp, Artificial Sequence
  Synthetic construct
acttgggtcattctggagttct

SEQ ID NO: 17 - DNA, 20 bp, Artificial Sequence
  Synthetic construct
cccactcctccacctttgac

SEQ ID NO: 18 - DNA, 20 bp, Artificial Sequence
  Synthetic construct
tcctcttgtgctcttgctgg

SEQ ID NO: 19 - RNA, 19 nt, Artificial Sequence
  Synthetic construct
gccagugauuauuuagauu

SEQ ID NO: 20 - RNA, 19 nt, Artificial Sequence
  Synthetic construct
gaacagcaccaaaggaguu

SEQ ID NO: 21 - RNA, 19 nt, Artificial Sequence
  Synthetic construct
cagauuagcacgaguuaaa

SEQ ID NO: 22 - RNA, 21 nt, Artificial Sequence
  Synthetic construct
cuucaagacuucaaugagauu

SEQ ID NO: 23 - RNA, 21 nt, Artificial Sequence
  Synthetic construct
cugcgaagguuggaaguaauu

SEQ ID NO: 24 - RNA, 21 nt, Artificial Sequence
  Synthetic construct
aucaagaacuccagaaugauu

SEQ ID NO: 25 - DNA, 24 bp, Artificial Sequence
  Synthetic construct
tgtgagagtgaggataatgacgaa

SEQ ID NO: 26 - DNA, 22 bp, Artificial Sequence
  Synthetic construct
ccaggaagggagtcaaatcaca

SEQ ID NO: 27 - DNA, 67 bp, Homo sapiens
gaagccaacaagaatttgagaactgtaaataccaagccttgaaagggaccatggtgcggcctgtgag

SEQ ID NO: 28 - DNA, 50 bp, Homo sapiens
acataagaaaccagtcaattactcacagtttgaccactctgacagtgatg

SEQ ID NO: 29 - DNA, 142 bp, Homo sapiens
atgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaag

SEQ ID NO: 30 - DNA, 110 bp, Homo sapiens
gatggctttagatgacaagctctaccagagagacttagaagttgcactagctttatcagtgaaggaacttccaacagtcaccactaatgtgcagaactctcaagataaaa

SEQ ID NO: 31 - DNA, 87 bp, Homo sapiens
gcattgaaaaacatggcagtagtaaaatagaaacaatgaataagtctcctcatatctctaattgcagtgtagccagtgattatttag

SEQ ID NO: 32 - DNA, 150 bp, Homo sapiens
atttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctg

SEQ ID NO: 33 - DNA, 165 bp, Homo sapiens
gtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttgg

SEQ ID NO: 34 - DNA, 107 bp, Homo sapiens
gtgaagattctgaggatgattctgatttttgtgagagtgaggataatgacgaagacttctctatgagaaaaagtaaagttaaagaaattaaaaagaaagaagtgaag

SEQ ID NO: 35 - DNA, 150 bp, Homo sapiens
tgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccag

SEQ ID NO: 36 - DNA, 1191 bp, Homo sapiens
cggcatctggaggtagcagaagtagcagcagcccactggtggtagtgtctgtgaagtctcccaatcagagtctccgccttggcttgtccagattagcacgagttaaacctttgcatccaaatgccactagcacctgagtgtggtacaggaggaatgtttggttgggagaatcacagctttacaagggtgtttatatttgatttgtgtttatatttgaggcaggtattgtaatataaaggaatccattaccatgtcctataaatgacctctagccattttatgattatgttctctgtaaaactcttcaagacttcaatgagaagtttgtttataagaattatcttctcatacctttccttgtgaagagcgtattctgtttttctatcagttcgacatgaagtccacatcacatgctgttcttttctagttacatgatgtgcctttctagctttgtctagtttatagcaccttaactttaactgttcagttttatctggcagaggaaaacattcttatttctttcagaagacatttctgaaatcttataagctacttaagctacgttgtcagttttatcgcaaagatgttttgtattttagccaaatctttttatagtacaaacttagaattattttacacactaaaatggttgcagttttatggcatatgtctccgatttagatggttattctctagaaaatagtatttaaagacattttatgaaatcttcattgtcaaaacctttaataaaagtggaaatattttgaaatgccctttttcttgataccactcatccacgtgttcctgattgtccacatttcatgataaaatgagagctccgcagagaatgttagcctttctgttgtaaatgtaatcttcaagtagtcactttttgttaagttctttagaaagtagttgtcaagtacttagtcatccctattatgatatgagatagtacagcttttcaggaagcttagatctgaatttactttgaaaaacaattgtaatgaatattttatatttacattgagaatttcaactagcttctgatcaatttttaataaaaaattttcaaatcatgttagctgttaaaaaatgtataataactcagtttttcttggtttatggaaatatctatattaatgtgaaaataattaatttagaattgtgattaaagtgagcatttgtcta

SEQ ID NO: 37 - DNA, 80 bp, Homo sapiens
tctcacagcctcccgcagcggcgggcggtcagcgccggcctcatgcagctcctcccgccgcctatccgcaccggaacaaa

SEQ ID NO: 38 - DNA, 94 bp, Homo sapiens
gactcaaatggatgctaaaaagccaaggaaatgtgatttgactcccttcctggttttgaaagcaagaaagaaacaaaagttcacctctgcgaag

SEQ ID NO: 39 - DNA, 81 bp, Homo sapiens
gttggaagtaaactttcggttcagatccagaagccaccttccaatatcaagaactccagaatgacccaagtctttcataag

SEQ ID NO: 40 - DNA, 111 bp, Homo sapiens
aacaccagtgttacttcactcccctttgtggacaccaaggggaagaagaatacggtaagcttcccacacattagcaagaaagtcctgctgaagtcatccctgctgtatcag

SEQ ID NO: 41 - DNA, 139 bp, Homo sapiens
gagaatcaagctcacaatcagatgccggcctcagagctcaaggcttcagaaatacctttccaccctagcattaaaacccaggatcccaaggcagaggagaagtcaccaaagaagcaaaaggtgactctgacagcggcag

SEQ ID NO: 42 - DNA, 164 bp, Homo sapiens
aggccctaaagctttttaagaaccagctgtctccatatgaacaaagtgaaatcctgggctacgcggagctgtggttcctgggtcttgaagccaagaagctcgacacggctcctgagaaatttagcaagacgagttttgatgatgagcatggcttctatctgaag

SEQ ID NO: 43 - DNA, 137 bp, Homo sapiens
gtcctgcatgatcacattgcctaccgctatgaagttctggagacaatcgggaaggggtcctttggacaggtggccaagtgcttggatcacaaaaacaatgagctggtggccctgaaaatcatcaggaacaagaagag

SEQ ID NO: 44 - DNA, 141 bp, Homo sapiens
gtttcaccagcaggccctgatggagctgaagatcctggaagctctcagaaagaaggacaaagacaacacctacaatgtggtgcatatgaaggactttttctactttcgcaatcacttctgcatcacctttgagctcctggg

SEQ ID NO: 45 - DNA, 139 bp, Homo sapiens
aatcaacttgtatgagttgatgaagaataacaactttcaaggcttcagtctgtccatagttcggcgcttcactctctctgttttgaagtgcttgcagatgctttcggtagagaaaatcattcactgtgatctcaagccc

SEQ ID NO: 46 - DNA, 82 bp, Homo sapiens
gaaaatatagtgctataccaaaagggccaagcctctgttaaagtcattgactttggatcaagctgttatgaacaccagaaag

SEQ ID NO: 47 - DNA, 173 bp, Homo sapiens
tatacacgtacatccaaagccggttctaccgatccccagaagtgatcctgggccacccctacgacgtggccattgacatgtggagcctgggctgcatcacggcggagttgtacacgggctaccccctgttccccggggagaatgaggtggagcagctggcctgcatcatggag

SEQ ID NO: 48 - DNA, 61 bp, Homo sapiens
gtgctgggtctgccgccagccggcttcattcagacagcctccaggagacagacattctttg

SEQ ID NO: 49 - DNA, 130 bp, Homo sapiens
attccaaaggttttcctaaaaatataaccaacaacagggggaaaaaaagatacccagattccaaggacctcacgatggtgctgaaaacctatgacaccagcttcctggactttctcagaaggtgtttggt

SEQ ID NO: 50 - DNA, 176 bp, Homo sapiens
atgggaaccttctcttcgcatgaccccggaccaggccctcaagcatgcttggattcatcagtctcggaacctcaagccacagcccaggccccagaccctgaggaaatccaattcctttttcccctctgagacaaggaaggacaaggttcaaggctgtcatcactcgagcagaaaag

SEQ ID NO: 51 - DNA, 360 bp, Homo sapiens
atgagatcaccaaagagactacagagaaaacaaaagatagccccacgaagcatgttcagcattcaggtgatcagcaggactgtctccagcacggagctgacactgttcagctgcctcaactggtagacgctcccaagaagtcagaggcagctgtcggggcggaggtgtccatgacctccccaggacagagcaaaaacttctccctcaagaacacaaacgttttaccccctattgtatgacctttgctgagggtatgtcctgctcctttccaccagtgatttgtattaagacagcacttatattgtacaatacttcagactgttttttttaaatacataaaactttatgttaaaaaactct

SEQ ID NO: 52 - DNA, 26 bp, Artificial Sequence
  Synthetic construct
gaaagaagtgaaggactcaaatggat

SEQ ID NO: 53 - DNA, 26 bp, Artificial Sequence
  Synthetic construct
tgggtcccaccaggactcaaatggat

SEQ ID NO: 54 - DNA, 26 bp, Artificial Sequence
  Synthetic construct
tgtaatgctttgggactcaaatggat

SEQ ID NO: 55 - DNA, 889 bp, Artificial Sequence
  Synthetic construct
caagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaaggatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttggtgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccag

SEQ ID NO: 56 - DNA, 681 bp, Artificial Sequence
  Synthetic construct
caagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaaggatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaag

SEQ ID NO: 57 - DNA, 771 bp, Artificial Sequence
  Synthetic construct
gaagccaacaagaatttgagaactgtaaataccaagccttgaaagggaccatggtgcggcctgtgagacataagaaaccagtcaattactcacagtttgaccactctgacagtgatgatgattttgtttctgcaactgtacctttaaacaagaaatccagaacagcaccaaaggagttaaaacaagataaaccaaaacctaacttgaacaatctccggaaagaagaaatcccagtacaagagaaaacccctaaaaaaaggatggctttagatgacaagctctaccagagagacttagaagttgcactagctttatcagtgaaggaacttccaacagtcaccactaatgtgcagaactctcaagataaaagcattgaaaaacatggcagtagtaaaatagaaacaatgaataagtctcctcatatctctaattgcagtgtagccagtgattatttagatttggataagattactgtggaagatgatgttggtggtgttcaagggaaaagaaaagcagcatctaaagctgcagcacagcagaggaagattcttctggaaggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttgg